(19) United States
(12) Patent Application Publication
(10) Pub. No.: US 2016/0146831 A1
(43) Pub. Date: May 26, 2016

(54) Antibody and Cytokine Biomarker Profiling for Determination of Patient Responsiveness

(71) Applicant: The Board of Trustees of the Leland Stanford Junior University, Stanford, CA (US)

(72) Inventors: WOLFGANG HUEBER, Menlo Park, CA (US); WILLIAM H. ROBINSON, Palo Alto, CA (US); LAWRENCE STEINMAN, Stanford, CA (US); PAUL J. UTZ, Portola Valley, CA (US); Mark C. Genovese, Sunnyvale, CA (US)

(21) Appl. No.: 14/947,815

(22) Filed: Nov. 20, 2015

Related U.S. Application Data
(63) Continuation of application No. 11/788,232, filed on Apr. 18, 2007.
(60) Provisional application No. 60/793,029, filed on Apr. 18, 2006.

(51) Int. Cl. G01N 33/68

(52) U.S. Cl. CPC G01N 33/6869; G01N 33/6863; G01N 2333/545; G01N 2333/5412; G01N 2333/5421; G01N 2333/525; G01N 2333/523; G01N 2333/522; G01N 2800/102; G01N 2800/52

(57) ABSTRACT

Compositions and methods are provided for prognostic classification of autoimmune disease patients into subtypes, which subtypes are informative of the patient's need for therapy and responsiveness to a therapy of interest. The patterns of circulating blood levels of serum autoantibodies and/or cytokines provides for a signature pattern that can identify patients likely to benefit from therapeutic intervention as well as discriminate patients that have a high probability of responsiveness to a therapy from those that have a low probability of responsiveness. Additionally, serum autoantibody and/or cytokine signature patterns can be utilized to monitor responses to therapy. Assessment of this signature pattern of autoantibodies and/or cytokines in a patient thus allows improved methods of care. In one embodiment of the invention, the autoimmune disease is rheumatoid arthritis.

This invention was made with Government support under contract N01-HV28183 awarded by the National Institutes of Health. The Government has certain rights in the invention.

### BACKGROUND OF THE INVENTION

There is a long-standing interest in manipulating cells of the immune system to achieve control of autoimmune disease. While targeted antigen-specific therapy remains of great interest, there has also been considerable development of polyclonal, or non-antigen specific therapies. In addition to general immunosuppression, e.g. through the use of agents such as hydrocortisone, many therapies are now being brought to the clinic that provide for a more selective modification of the immune system, such as blockade of cytokines such as TNFα, IL-1, IL-6, and IL-15; reduction of B cell populations, T cell populations; or altering interactions of adhesion or signaling molecules prominent in inflammation.

While overall reduction of T lymphocytes has led to disappointing clinical results, including long-lasting lymphopenia, recently interest has focused on modulating T cell function rather than depleting large number of T cells or subsets of T cells. The important role of co-stimulation in the activation of T cells is now well understood, and this process has been targeted therapeutically with the cytotoxic T lymphocyte-associated antigen 4-Ig (CTLA4Ig) fusion protein, which interferes with co-stimulation without depletion of T cells. Other agents, such as cyclosporine A, interfere with T cell signaling pathways.

B cells are responsible for producing autoantibodies, even in diseases thought to have a largely T cell pathology, for example, rheumatoid factors (RF) and other RA-associated autoantibodies such as anti-cyclical citrullinated peptide (CCP) antibodies. B cells also act as highly efficient antigen-presenting cells (APC) to T cells and thus may play an important role in T cell activation. A number of approaches are now available for reducing B cell populations, e.g. anti-CD20, and have demonstrated efficacy in treating rheumatoid arthritis and other autoimmune diseases.

Cytokines are messenger molecules produced by B cells, T cells, macrophages, dendritic cells and other immune and host cells. Cytokines play roles in the pathogenesis of rheumatoid arthritis, multiple sclerosis and other autoimmune diseases. Cytokines include chemokines, interleukins, lymphokines, growth factors, angiogenesis factors, and other secreted and cell surface molecules that transmit signals to other cells. Cytokines include, but are not limited to, TNFα, INFγ, IL-1, IL-2, IL-4 IL-6, IL-8/CXCL8 IL-10, IL-12, IL-13, IL-15, IL-17, IL-18, IL-23, RANTES/CCL5, IP-10/CXCL10, eotaxin/CCL11, MCP-1/CCL2, MIP-1α/CCL4, growth factors such as GM-CSF, VEGF, PDGF, IGF; other secreted molecules include proteases such as metalloproteinases (MMPs), and their tissue inhibitors (TIMPs). Blockade of several of these with biological agents (monoclonal antibodies and soluble receptors), including TNFα (with etanercept, infliximab and adalimumab), IL-1 (with Anakinra) and IL-6 (Tocilizumab, currently in trials), have already provided therapeutic benefit in autoimmune diseases.

A number of chemotherapeutic approaches that target replicating cell populations, (which include lymphocytes), have also been used in the treatment of autoimmune disease. For example the administration of methotrexate, cyclophosphamide, mycophenolate mofetil, azathioprine, and the like, have been effective for certain patient populations.

A downside to these promising therapies is the diversity of responses in patient populations. While a significant proportion of patients may respond to a particular therapy, many do not. The clinician may therefore need to prescribe sequential expensive and time-consuming therapies in order to determine which is effective for the individual patient.

The use of disease-modifying therapies in autoimmune conditions is of great clinical interest, however these therapies suffer from the inability to determine a priori which patients will benefit. The present invention addresses this need.

### PUBLICATIONS

Autoantibody profiles and uses thereof are described in U.S. Patent application, publication US-2003-0003516-A1, herein incorporated by reference.

### SUMMARY OF THE INVENTION

Compositions and methods are provided for prognostic classification of individuals into groups that are informative of the individual's responsiveness to a therapy of interest. The levels of circulating serum autoantibodies and/or cytokines identified herein provides for a specific signature pattern, which when present distinguishes individuals who have a high probability of responsiveness to a therapy from those who have a low probability of responsiveness. Assessment of this signature pattern of autoantibodies and/or cytokines in a patient thus allows improved care.

In some embodiments of the invention, methods of determining an autoantibody signature pattern in a patient with an immune-related disease comprise: preparing an autoantigen panel comprising a plurality of autoantigens; physically contacting the antigen panel with a patient sample comprising antibodies; identifying the autoantibodies that bind to autoantigens within the panel; comparing the antibodies bound to the autoantigens with a control sample known to be associated with responsiveness or non-responsiveness to a therapy. Autoantigens of interest include proteins, peptides, modified proteins and peptides, proteoglycans, polynucleotides, lipids, carbohydrates, and the like. Protein and peptide modifications include but are not limited to citrullination (deimination), phosphorylation, glycosylation, ubiquitination, lipidation and methylation. Heterophilic antibodies, e.g. Rheumatoid Factor (RF), etc. are optionally depleted or blocked in a sample prior to analysis, for example by the addition of a blocking agent to attenuate non-specific cross-linking of capture and detection antibodies by RF.

In other embodiments of the invention, methods of determining a cytokine signature pattern in a patient with an immune-related disease comprise: preparing a cytokine measurement panel comprising a plurality of antibodies against cytokines; physically contacting the anti-cytokine antibody panel with a patient sample comprising cytokines; identifying the cytokines that bind to antibodies within the panel; comparing the cytokines bound to the those bound with a control sample known to be associated with responsiveness or non-responsiveness to a therapy. The resulting data set provides a signature pattern from which the prognosis can be determined. Cytokines of interest include, but are not limited to, TNFα, INFγ, IL-1α, IL-1β, IL-2, IL-6, IL-8/CXCL8, IL-10, IL-12p40, IL-15, IL-17, IL-18, IL-23, MCP-1/CCL2, IP-10/CXCL10, RANTES/CCL5 and GM-CSF.

In one embodiment of the invention, the autoimmune disease is rheumatoid arthritis. Disease modifying anti-rheumatoid drugs (DMARD) of interest include, without limitation, cytokine blocking agents, e.g. anti-TNFα antibodies, soluble TNFα receptor, soluble IL-1 receptor (Anakinra), and anti-IL-6R antibodies (Tocilizumab); T cell targeted therapies (CTLA4-Ig [Abatacept]), B cell targeted therapies (anti-CD20 [Rituximab]), chemotherapeutic drugs, and the like.

In another embodiment, prognostic algorithms are provided, which combine the results of multiple autoantibody and/or cytokine level determinations and/or other clinical and laboratory parameters, and which will discriminate between individuals who will respond to the therapy of interest, and those who will not respond. In one embodiment of the invention, antibody binding to a panel of autoantigens and cytokine binding to a panel of antibodies is evaluated. In other embodiments autoantibody signature patterns and cytokine signature patterns are analyzed in combination with clinical, imaging, laboratory and genetic parameters to assess an individual patient's disease state and thereby determine if they would benefit from initiation of therapy. The use of such panels can provide a level of discrimination not found with individual epitopes or singular antibodies or cytokines.

In one use of such an algorithm, a reference dataset is obtained, which comprises, as a minimum, autoantibody binding profiles and cytokine levels to at least one, and usually a panel of autoantigens and cytokines identified herein. Such a database may include positive controls representative of disease subtypes, for example anti-TNFα Responder, anti-TNFα Non-Responder, etc.; and may also include negative controls, e.g. measurements of serum antibodies and cytokines in normal human serum. The dataset optionally includes a profile for clinical indices; additional protein signature patterns; metabolic measures, genetic information, and the like. The autoimmune disease dataset is then analyzed to determine statistically significant matches between datasets, usually between reference datasets and test datasets and control datasets. Comparisons may be made between two or more datasets.

Methods of analysis may include, without limitation, establishing a training dataset, and comparing the unknown sample to the training dataset as test datasets. Alternatively, simple quantitative measure of a panel of autoantigens and cytokines may be performed, and compared to a reference to determine differential expression. Other methods, examples of which are included in one embodiment, may utilize decision tree analysis, classification algorithms, regression analysis, principal components analysis, multivariate analysis, predictive models, and combinations thereof.

In other embodiments of the invention a device or kit is provided for the analysis of patient samples. Such devices or kits will include reagents that specifically identify one or more autoantibodies and/or cytokines, where at least a subset of cytokines and autoantibodies are selected from Tables 1 and 2, respectively. Devices of interest include arrays, where the reagents are spatially separated on a substrate such as a slide, gel, multi-well plate, etc. Alternatively the reagents may be provided as a kit comprising reagents in a suspension or suspendable form, e.g. reagents bound to beads suitable for flow cytometry, and the like. Reagents of interest include reagents specific for autoantibody markers. Such reagents may include antigenic proteins or peptides, and the like. Such devices or kits may further comprise cytokine-specific antibodies or fragments thereof; and the like.

### BRIEF DESCRIPTION OF THE DRAWINGS

FIG. 1. Synovial antigen array characterization of autoantibody responses in RA. Synovial arrays were produced by printing over 200 distinct protein and peptides antigens representing candidate autoantigen targets in RA, including: collagens type I, II, III, IV, VI, IX and XI; GP-39 (glycoprotein 39 kDa) and overlapping peptides derived from GP-39; BiP; native and citrullinated fibrinogen and vimentin, protein and overlapping peptides; native and citrulline-substituted cyclic filaggrin peptides such as “CCP1” (also known in the literature as cyc 0112-15) and “CCP11” (also known in the literature as cyc Ala-6); hnRNP-B1 and -D; GPI; and heat shock proteins 65, 70, 90 (Sigma) and BiP. Arrays were probed with 1:150 dilutions of serum from 2 RA patients. The light features are marker features to orient the arrays. RA-1 reacted with protein antigens such as citrullinated fibrinogen, citrullinated vimentin, BiP, hnRNP-B1 and hnRNP-D, as well as multiple peptides, including “CCP1” and “CCP 11” and “hnRNPB1 pep” while RA-2 lacks reactivity against these peptides but possesses additional reactivity against the enzyme antigen GPI (glucose-6-phosphate isomerase).

FIG. 2. Autoantibody targeting of citrullinated epitopes delineates a subpopulation of RA patients. Synovial antigen arrays were used to determine autoantibody reactivity in 18 RA and 38 control serum samples obtained from the Stanford Arthritis Center sample bank. The image represents hierarchical clustering of patients and antigen features. SAM was used to determine antigen features with statistically significant differences in array reactivity between the RA and control patients (false discovery rate (FDR)<0.035 for the reported list). A hierarchical clustering algorithm was used to order patient samples based on similarities in their SAM-determined array feature reactivities (the dendrogram above the image represents the cluster relationships), and to order SAM-determined array features based on similarities in reactivities in the patient samples examined (dendrogram to right). The tree dendrograms represent the relationships between patient samples or antigen features, with branch lengths representing the extent of similarities in array reactivity determined by the cluster algorithm. Following clustering, labels were added below the images to indicate the general locations of clusters of RA and control patients.

FIG. 3. Synovial antigen array validation. Comparison of array reactivity against strongest reactive CCP and commercial CCP2 ELISA results for detection of anti-citrulline autoantibodies in RA patients derived from the Arthritis, Rheumatism, and Aging Medical Information System (ARAMIS) inception cohort. Dark dots represent samples negative by CCP2 ELISA, and light dots represent samples positive by CCP2 ELISA.

FIG. 4. Autoantibody targeting of citrullinated epitopes in patients with early RA is predictive for more severe disease. Pairwise SAM analysis was performed to identify antigen features with significant differences (FDR<0.07) in synovial array reactivity associated with laboratory and clinical parameters previously identified to provide diagnostic and prognostic value. The specific analysis shown in the image is a comparison of female rheumatoid factor-seropositive RA patients (samples obtained within 6 months of the diagnosis of RA) with serum C-reactive protein (CRP) levels 0.5 mg/dl (low inflammation, characterizing patients likely to develop less severe disease) and 1.5 mg/dl (high inflammation, characterizing patients likely to develop more severe disease), respectively. Hierarchical clustering was applied to arrange the patients and SAM-identified antigen features. The labels below the cluster image indicate the general locations of patients within respective groups. The labels to the right of the cluster images indicate the locations of citrullinated antigens (upper box) and native antigens (lower box). Thus, antigen microarray profiling of autoantibodies demonstrates that autoantibodies targeting citrullinated epitopes are associated with features predictive for the development of severe RA, while autoantibodies targeting native epitopes, including several human cartilage glycoprotein 39 peptides and collagen type II, are associated with predictors of less-severe RA.

FIG. 5A-5C. Impact of immunoglobulin depletion (depletion of rheumatoid factor [RF]) on the quantification of cytokines by multiplex assay. Serum samples from 14 patients with established RA (9 RF seropositive, 5 RF seronegative) were either depleted of immunoglobulins by immunoprecipitation using protein L-sepharose beads, or used untreated, followed by analysis on the multiplex cytokine assay. Representative results are shown for (FIG. 5a) IL-4, (FIG. 5b) TNFα, and (FIG. 5c) IL-10. Concentrations are shown on a logarithmic scale on the left, RF seropositive and RF seronegative samples are labeled on the bottom of each panel, light columns represent measurements in immunoglobulin-depleted serum, dark columns represent measurements in undepleted serum. These data demonstrate that in certain samples RF can result in false elevations in blood cytokine readouts, and that depletion of RF can greatly reduce such false positive elevations to enable true measurements of blood cytokines.

FIG. 6A-6C. Development and optimization of methods for cytokine profiling: use of rheumatoid factor (RF) blocking agents to prevent false elevations in blood cytokine readouts. Serum samples from 4 RA patients with RF seropositive and 2 patients with RF seronegative RA were analyzed by multiplex cytokine assay. Serum samples were either untreated (native), or pre-treated by: (i) incubation with protein L-sepharose beads to remove immunoglobulin; and (ii) a blocking agent (HeteroBlock™, “HB”) at 1:175 dilution to attenuate non-specific cross-linking of capture and detection antibodies by RF, followed by sample analysis on the multiplex cytokine assay. Each dot represents an individual sample. Concentrations in ng/ml are indicated on a linear scale on the left of each graph. Different sample treatment groups are labeled below the respective columns. RF, rheumatoid factor; Ig, immunoglobulin; ProtL, protein L-sepharose beads; HB, Heteroblock™. The optimized conditions using Heteroblock minimize false elevations in blood cytokine readouts, and these optimized methods were used to generate the data presented in all of the subsequent Figures.

FIG. 7. Serum cytokine profiles stratify early RA patients (<6 months disease duration) into high and low inflammatory subtypes predictive for future development of severe versus mild arthritis, respectively. We applied bead-based arrays (Luminex System using XMap bead array technology) to simultaneously profile cytokines in serum samples derived from 56 patients in an early RA inception cohort (disease duration <6 months). Array results are displayed as a heat map after hierarchical clustering of all data points to visualize the spectrum of cytokine levels for each patient. Columns represent individual patients, labeled on the top. The scale of cytokine levels is provided at the upper right. For each patient, the number of copies of the shared epitope (SE) major histocompatibility complex (MHC) polymorophism (0, 1 or 2 copies), RF status (positive or negative), and cyclic-citrullinated peptide 2 (CCP2) ELISA reactivity (positive or negative) are indicated across the top of the panel. Rows representing individual cytokine levels are labeled on the right side of the panel. These data are from experiments performed utilizing HeteroBlock™ at a 1:175 dilution to minimize the effects of RF and other heterophilic antibodies that could cause false elevations in cytokine readouts in this assay. The cytokine-high subgroup of RA patients cluster on the left side of the heatmap image, exhibit broad elevations in multiple cytokines, represent approximately ⅓ of patients in this RA patient cohort, and are associated with laboratory (positive RF and anti-CCP) and genetic (possession of the SE polymorphism) features predictive for severe arthritis (samples 77, 278, 111, 613, 294, 216, 194, 185, 108, 114, 197, 333, 196, 253, 616, 369, and 203). In contrast, the cytokine-low subgroup of RA patients cluster on the right side of the heatmap, do not exhibited elevations in blood cytokines, represent approximately ⅔ of patients in this cohort, and are associated with laboratory (negative RF and anti-CCP) and genetic (lack of the SE polymorphism) features predictive for a less severe disease course. IL=interleukin; GM-CSF=granulocyte macrophage colony stimulating factor; MIP-1α=macrophage inhibitory protein 1alpha, MCP-1=monocyte chemoattractant protein 1.

FIG. 8A-8H. Comparison of cytokine concentrations in healthy individuals, patients with psoriatic arthritis (PsA) and ankylosing spondylitis (AS), and patients with early RA (ERA). Serum samples from patients with early RA (n=56), PsA and AS (n=21) and from healthy subjects (n=19) were analyzed by the Luminex bead array system using optimized methods with Heteroblock (per FIG. 6). Results are shown for (FIG. 8a) IL-1α, (FIG. 8b) IL-6, (FIG. 8c) TNFα, (FIG. 8d) IL-12p40, (FIG. 8e) IP-10/CXCL10, (FIG. 8f) Eotaxin/CCL11, (FIG. 8g) MCP-1/CCL2, and (FIG. 8h) IL-8/CXCL8. Horizontal bars represent medians with percentiles for each column. P-values were calculated by Kruskal-Wallis tests with Dunn's multiple comparisons, and comparisons with p-values <0.05 are indicated by “*”, <0.01 by “***”, and non-significant values by “n.s.”.

FIG. 9A-9B. Profiles of blood antibodies targeting citrullinated and native epitopes identify a subgroup of RA patients that subsequently respond to therapy with the anti-TNFα drug etanercept (ENBREL™). Arthritis arrays were used to determine autoantibody profiles in blood samples derived from RA patients prior to treatment with etanercept. Responder status was determined based on the American College of Rheumatology response criteria. For this experiment Responders (R) were selected based on exhibiting a significant ACR response to etanercept (ACR50 or greater in A [denoted by “R baseline”]; ACR40 or greater in B [with the degree of response given, e.g. “ACR40”, “ACR50”, “ACR60”, “ACR70”, etc.), while Non-Responders (NR) were selected based on exhibiting a minimal or no response (ACR20 or worse, in both A and B). The Significance Analysis of Microarrays (SAM) algorithm was applied to identify antibody reactivities with statistically significant differences between Non-Responders and Responders (from the 500+ antigens included on the arrays), and the significant antigen lists are presented to the right of the heatmaps (the false discovery rate (FDR) for individual antigens was set at <0.03 for both experiments A and B). In both unsupervised hierarchical clusters (A and B), Non-Responders cluster on the left side of the heatmap and Responders on the right side of the heatmap. The heatmaps demonstrate that Responders possess increased autoantibody targeting of multiple citrullinated and native epitopes in their baseline blood samples (prior to etanercept therapy). The tree dendrograms represent the relationships between patient samples or antigen features, with branch lengths representing the extent of similarities in array reactivity determined by the cluster algorithm. FIG. 9A, Analysis comparing pre-treatment baseline autoantibody profiles of etanercept Non-Responders with Responders. FIG. 9B, An independent experiment in a larger number of patients comparing baseline (pre-treatment) autoantibody profiles in etanercept Non-Responders with Responders.

FIG. 10. Identification of blood autoantibody profiles that predict subsequent response to etanercept therapy in patients with RA. Prediction analysis of microarrays (PAM) was applied to identify autoantibody profiles in synovial antigen array datasets derived from baseline (pre-treatment) serum samples that predict subsequent response to etanercept therapy. The presence of autoantibodies targeting 6 peptides out of >500 antigens included on synovial antigen microarrays were identified by PAM to best classify etanercept Responders from non-Responders at the selected threshold of 1.5 (indicated as a vertical line in the graph to the right) in this specific patient cohort. The 6 antigens for which predictive antibodies were identified are listed at the bottom of the figure, citrullinated epitopes are highlighted, and their corresponding peptide sequences are provided in Table 1. Based on autoantibody reactivities against these 6 peptide epitopes, in the confusion matrix (re-randomized samples) PAM correctly classified 10 out of 14 Responders (ACR50 or greater, 71%) and 13 out of 15 Non-Responders (ACR20 or worse, 87%).

FIG. 11A-11F. Enzyme-linked immunosorbent assay (ELISA) validation of autoantibody reactivities against a subset of peptide and protein antigens that were identified by SAM and PAM to differentiate etanercept Responders from Non-Responders. ELISA was utilized to detect autoantibodies in pre-treatment sera derived from etanercept Non-Responders (NR; ACR 20 or less; left columns) and Responders (R; ACR50 or greater; right columns) for 6 selected peptide and protein antigens. 43 etanercept-treated patients from an independent patient cohort from that described in FIGS. 9A-9B and 10 were analyzed in this analysis. Respective p-values are indicated at the bottom of each panel. These data provide ELISA validation for a subset of the SAM and PAM identified autoantibody reactivities that differentiate etanercept Rs from NRs.

FIG. 12A-12F. Increased levels of blood cytokines are present in pre-treatment sera from a subset of anti-TNF etanercept responders. The Luminex bead array system and optimized conditions (FIG. 6A-6C) were utilized to profile cytokines and chemokines in a cohort of 43 patients treated with etanercept. Comparisons of six individual cytokines and chemokines are demonstrated. In each individual panel, serum cytokine expression of Non-Responders (NR) is shown in the left columns and cytokine expression of Responders (R) is shown in the right columns. Horizontal bars indicate median serum expression levels. P-values derived from 2-sided t-tests are indicated at the bottom of the images. Elevated levels of IL-6, IL-1α, eotaxin and GM-CSF best classified subsets of etanercept Responders from Non-Responders.

FIGS. 13A and 13B. Multi-Dimensional Scaling (MDS) analysis identifies blood autoantibodies and a cytokine that differentiate etanercept Responders from Non-Responders. Thirty-four biomarkers previously identified by (FIG. 13a) synovial antigen microarray & ELISA analysis of autoantibodies, and (FIG. 13b) Luminex bead array analysis of cytokines, as providing the greatest predictive value for differentiating etanercept Responders from Non-Responders were analyzed by MDS. Forty-three etanercept-treated patients were first analyzed by regression analysis for differential targeting of peptide autoantigens in Responders (R) and non-Responders (NR). Eight out of 20 peptide antigens demonstrated differential regulation with p-values of 0.05 or less: acetyl-calpastatin peptide 184-210, ApoE277-296cit, Fibromodulin 246-265, PG4 1184-2003, FibrinogenA616-635cit, Serine Protease II 433-452, Clusterin 386-405cit, H2B1-20. FIG. 13A, Square root-transformed data of four of these eight antigens were then used for the examples in the figure: p1 (acetyl calpastatin peptide 184-210), p2 (ApoE277-296cit), p20 (Osteoglycin 177-196), GM-CSF. Full diamonds=Non-Responders; Open circles=Responders. A subset of the responders localize in the statistical areas indicated by the arrows, and thus are characterized by the this profile of peptide-specific autoantibodies and the cytokine GM-CSF. FIG. 13B, Display of the recursive partitioning data from A as a decision tree. Classification of pre-treatment samples from etanercept-treated patients is demonstrated, based on differential serum levels of 3 biomarkers including: (1) p1 (acetyl-calpastatin peptide 184-210; threshold value of 0.52), (2) the cytokine GM-CSF (threshold value of 0.45), and (3) p20 (Osteoglycin 177-196 peptide, threshold value of 0.22). Thus, 36 out of 43 samples in this specific cohort were classified correctly by recursive partitioning on P1, then GM-CSF, then P20 at the indicated thresholds. The 7 remaining samples could not be correctly classified with these 3 parameters.

FIG. 14A-14C. Characterization of the high-inflammatory severe subtype of RA: Elevated levels of blood cytokines are associated with autoantibodies targeting multiple citrullinated epitopes. Autoantibody reactivity was determined by antigen arrays and cytokine concentrations were determined by the Luminex bead array multiplex cytokine assay in 56 early RA serum samples. Pairwise SAM was performed to identify antigen features with statistically significant differences in arthritis array reactivity that were associated with elevated levels of serum cytokines. Specific analyses include comparisons of RA patients who had elevated versus unmeasurable serum levels of IL-18 (FIG. 14A), GM-CSF (FIG. 14B), and TNF-α (FIG. 14C), with upper cut-off thresholds being the 75^{th} percentile for the cytokine^{high} group. Hierarchical clustering was applied to arrange the patients and SAM-identified antigen features (dendrograms on the top and right, respectively). The labels below the cluster images indicate the general locations of patients of the respective cytokine group. Citrullinated antigens/epitopes are shown in light type and native antigens in dark (black) type.

FIG. 15. Response to anti-TNFα therapy with etanercept (ENBREL®) treatment is associated with decrease in blood autoantibody reactivity and cytokine levels. Comparison of antibody and cytokine profiles in serum derived from RA patients who responded (ACR50 or greater response) to etanercept. Antibody profiling using synovial arrays and cytokine profiling using the multiplex bead array was performed on baseline samples (obtained pre-etanercept) and 3-months following the initiation of etanercept therapy. SAM was performed to identify blood antibodies and cytokines with differences in reactivity between the baseline and 3-month timepoints (FDR<0.33), patients and antigens subjected to hieratical cluster analysis and displayed as a heatmap. This figure demonstrates reductions in autoantibodies targeting multiple native and citrullinated antigens, as well as a reduction in IL-6 levels, following 3 months of etanercept treatment in ACR50 or greater Responders.

### DETAILED DESCRIPTION OF THE EMBODIMENTS

Compositions and methods are provided for prognostic classification of autoimmune disease patients (a) according to initial disease severity and long-term clinical outcome, and (b) according to their ability to respond to disease modifying therapy using an antibody and cytokine signature patterns. Antibody signature pattern as used herein refers to the antigen or epitope spectrum of antigens or epitopes recognized by the antibodies derived from a patient sample, e.g. as determined by array. Cytokine signature pattern as used herein refers to the spectrum of cytokine levels as determined by an antibody binding assay. Once the subset of antibody specificities and/or cytokine levels for a particular sample are identified, the data is used in selecting the most appropriate therapy for an individual. By analysis of autoantibody specificity and/or cytokine levels on an individual basis, the specific subclass of disease is determined, and the patient can be classified based on: (i) the predicted severity of disease, and thereby the need for therapy as well as the potency of the therapy warranted, and (ii) as to the likelihood to respond to anti-TNF or other treatments of interest. Thus, the signature patterns of autoantibodies and/or cytokines can provide prognostic information to guide clinical decision making, both in terms of institution of and escalation of therapy as well as in the selection of the therapeutic agent to which the patient is most likely to exhibit a robust response.

Various techniques and reagents find use in the diagnostic methods of the present invention. In one embodiment of the invention, blood samples, or samples derived from blood, e.g. plasma, serum, etc. are assayed for the presence of specific autoantibodies. Typically a blood sample is drawn, and a derivative product, such as plasma or serum, is tested. Such antibodies may be detected through specific binding members. Various formats find use for such assays, including autoantigen arrays; ELISA and RIA formats; binding of labeled peptides in suspension/solution and detection by flow cytometry, mass spectroscopy, and the like. Detection may utilize one or a panel of autoantigens, preferably a panel of autoantigens, for example in an array format. Cytokine detection may utilize a panel of antibodies specific for a spectrum of cytokines. Autoantibody and/or cytokine signature patterns typically utilize a detection method coupled with analysis of the results to determine if there is a statistically significant match with a pre-determined signature pattern of interest.

In one embodiment of the invention, the autoimmune disease is rheumatoid arthritis. Disease modifying anti-rheumatoid drugs of interest include, without limitation, anti-TNF agents, e.g. antibodies, receptors, etc., T cell targeted therapies, B cell targeted therapies, chemotherapeutic drugs, and the like. In one embodiment of the invention, the panel of autoantigens includes citrullinated proteins or peptides. Analysis may include one or more epitopes from a distinct protein, for example as shown in Table 1. Proteins of interest include, without limitation, fibromodulin, vimentin, collagen type II, HCgp39, fibrinogen, biglycan, decorin, aggrecan, calpastatin, clusterin, COMP, lumican, osteoglycin, ApoE, HSP90, HSP65, dnaJ, and histone 2A and 2B. Epitopes within proteins of interest may include citrullinated and/or native forms of peptides or proteins. Analysis may also include one or more cytokines, for example as shown in Table 2. The analysis will generally include at least about two epitopes and/or cytokines as set forth in Tables 1 and 2, and some types of analysis will usually include at least about ten epitopes and/or cytokines, and some types of analysis at least about 15, at least about 20 or more of the epitopes and/or cytokines. In some embodiments of the invention, the analysis will include at least about 6, at least about 8, at least about 10 or all of the epitopes selected from the group consisting of hFibA41-60cit; Vim58-77cit; biglycan 247-266; clusterin 221-240; acetyl-calpastatin peptide 184-210; ApoE277-296cit; Fibromodulin 246-265; PG4 1184-2003; FibrinogenA616-635cit; Serine Protease II 433-452; Clusterin 386-405cit and H2B1-20.

The panel of autoantigens may comprise discrete protein complexes, whole proteins and/or fragments of proteins, where the fragments may be overlapping peptides that encompass the complete protein, or a partial representation of the protein, which may include known immunodominant epitopes. The array for profiling antibodies may also comprise discrete molecules including single stranded DNA, double stranded DNA, oligonucleotides, RNA, lipids, carbohydrates, aptamers, peptoids, other molecular mimics and the like.

Cytokines may be measured using a panel of antibodies against cytokines, mass spectrometry or with other cytokine detection methods. Panels of anti-cytokine antibodies can be used to measure cytokines in assay formats such as ELISA, fluorescent immunoassays, antibody array technologies, bead array technologies, radioimmunoassay (RIAs), surface plasmon resonance-based detection technologies, and other immunoassay methodologies.

The information obtained from the antibody specificity and/or cytokine profile is used to (a) determine type and level of therapeutic intervention warranted (i.e. more versus less aggressive therapy, monotherapy versus combination therapy, type of combination therapy)), and (b) to optimize the selection of therapeutic agents. With this approach, therapeutic regimens can be individualized and tailored according to the specificity data obtained at different times over the course of treatment, thereby providing a regimen that is individually appropriate. In addition, patient samples can be obtained at any point during the treatment process for analysis.

Mammalian species that provide samples for analysis include canines; felines; equines; bovines; ovines; etc. and primates, particularly humans. Animal models, particularly small mammals, e.g. murine, lagomorpha, etc. may be used for experimental investigations. Animal models of interest include those for models of autoimmunity, graft rejection, and the like.

### Autoantigens

Antigens include molecules such as nucleic acids, lipids, carbohydrates, proteoglycans ribonucleoprotein complexes, protein complexes, proteins, glycoproteins, polypeptides, peptides, lipids, glycolipids, and naturally occurring or synthetic (in vitro) modifications of such molecules against which an immune response involving T and B lymphocytes can be generated. For each antigen, there exists a panel of epitopes that represent the immunologic determinants of that antigen. Antigens include any molecule that can be recognized, all or in part, by an antibody or T cell receptor.

Autoantigens are any molecule produced by the organism that are the target of an immunologic response, including lipids, carbohydrates, nucleic acids, peptides, polypeptides, and proteins encoded within the genome of the organism. Such molecule also include post-translationally-generated modifications of these peptides, polypeptides, and proteins, such as cleavage, phosphorylation, glycosylation, deimination of arginine to citrulline, and other modifications generated through physiologic and non-physiologic cellular processes. Such molecules also include modifications of these biomolecules, such as oxidation, cleavage products, and degradation products that result from both physiologic and pathologic processes. In certain cases such molecules can arise from degenerative processes, as a result of inflammatory processes, as a result of environmental factors and stimuli (such as tobacco smoking, pollutants, allergens, foods, etc.), or as a result of viral or bacterial infections. Viral and bacterial infections are well established to modify certain host proteins and other biomolecules as well as to stimulate potent immune responses which in certain cases may target host proteins resulting in an autoimmune disease.

Epitopes are portions of antigens that are recognized by antibodies or T cell antigen receptors. An individual antigen typically contains multiple epitopes, although there are instances in which an antigen contains a single epitope. In one embodiment of this invention, peptide fragments derived from a whole protein antigen are used to represent individual epitope(s) targeted by the antibodies produced by B cells. In another embodiment, portions of molecules representing post-translational modifications, carbohydrates, lipids and other molecules can be used to represent individual epitopes. Epitopes represent shapes recognized by immune B and T cells, and can also be represented by non-antigen derived peptides and other molecules that possess the same epitope shape that is present within the native antigen. An example of an element with an epitope shape is an aptamer. An aptamer is a molecule that provides a shape that can mimic an immunologic epitope. Using a plurality of aptamers a library of epitope shapes can be generated. Where peptides are used as an epitope to detect antibody binding, peptides will usually be at least about 7 amino acids in length, may be at least about 15 amino acids in length, and as many as 22 amino acids in length. The peptides of a protein may be overlapping by 5-10 amino acids, and can encompass the whole sequence of a protein of interest.

For analysis of rheumatoid arthritis patients, the antigens listed in Table 1 are of interest, both as reactive species and internal controls for certain reactive species.

TABLE 1
Antigens and peptide epitopes of interest for identifying
autoantibody profiles in rheumatoid arthritis (list
includes both reactive species and controls).

| SEQ | | | |
|---|---|---|---|
| ID | | | |
| NO | Antigen name | Protein | Sequence (cit = citrulline) |
| 1 | H2A/x 33-52 cit | histone H2A | [Cit]LL[Cit]KGHYAE[Cit]VGAGAPVYL |
| 2 | H2A 63-82 cit | histone H2A | ILELAGNAA[Cit]DNKKT[Cit]IIP[Cit] |
| 3 | H2A/a 1-20 | histone H2A | MSGRGKQGGKARAKAKTRSS |
| 4 | H2A/a 1-20 cit | histone H2A | MSG[Cit]GKQGGKA[Cit]AKAKT[Cit]SS |
| 5 | H2A/x 1-20 | histone H2A | MSGRGKTGGKARAKAKSRSS |
| 6 | H2A/x 1-20 cit | histone H2A | MSG[Cit]GKTGGKA[Cit]AKAKS[Cit]SS |
| 7 | H2A/x 33-52 | histone H2A | RLLRKGHYAERVGAGAPVYL |
| 8 | H2A33-52 | Histone H2A | RLLRKGNYAERVGAGAPVYL |
| 9 | H2B/f 1-20 | histone H2B | MPEPSKSAPAPKKGSKKAIT |
| 10 | H2B/a 16-35 | histone H2B | KKAVTKAQKKDGKKRKRSRK |
| 11 | H2B 1-20 | Histone H2B | MPEPVKSAPVPKKGSKKAIN |
| 12 | H2B 77-96 | Histone H2B | EASRLAHYNKRSTITSREIQ |
| 13 | hFibA41-60 | Fibrinogen alpha | GGGVRGPRVVERHQSACKSDS |
| 14 | hFibA61-80 | fibrinogen alpha | NYKCPSGCRMKGLIDEVNQD |
| 15 | hFibA211-230 | fibrinogen alpha | DLLPSRDRQHLPLIKMKPVP |
| 16 | hFibA121-140cit | fibrinogen alpha | NN[CIT]DNTYN[CIT]VSEDL[CITS[CIT]IEV |
| 17 | hFibA211-230cit | fibrinogen alpha | DLLPS[CIT]D[CIT]QHLPLIKMKPVP |
| 18 | hFibA571-590cit | fibrinogen alpha | PS[CIT]GKSSSYSKQFTSSTSYN |
| 19 | hFibA166-185 | fibrinogen alpha | MKRLEVDIDIKIRSCRGSCS |
| 20 | hFibA226-245 | fibrinogen alpha | MKPVPDLVPGNFKSQLQKVP |
| 21 | hFibA466-485 | fibrinogen alpha | TKTVIGPDGHKEVTKEVVTS |
| 22 | hFibA511-530 | fibrinogen alpha | HRHPDEAAFFDTASTGKTFP |
| 23 | hFibA586-605 | fibrinogen alpha | STSYNRGDSTFESKSYKMAD |
| 24 | hFibA31-50cit | fibrinogen alpha | GGGV[CIT]GP[CIT]VVE[CIT]HQSACKDS |
| 25 | hFibA41-60cit | Fibrinogen alpha | GGGV[CIT]GP[CIT]VVE[CIT]HQSACKDS |
| 26 | hFibA256-275cit | fibrinogen alpha | QM[CIT]MELE[CIT]PGGNEIT[CIT]GGST |
| 27 | hFibA286-305cit | fibrinogen alpha | P[CIT]NPSSAGSWNSGSSGPGST |
| 28 | hFibA586-605cit | fibrinogen alpha | STSYN[CIT]GDSTFESKSYKMAD |
| 29 | hFibA616-635 cit | Fibrinogen alpha | THSTK[CIT]GHAKS[CIT]PV[CIT]GIHTS |
| 30 | hFibB16-35 | Fibrinogen beta | KHLLLLLLCVFLVKSQGVND |
| 31 | hFibB46-65cit | Fibrinogen beta | H[CIT]PLDKK[CIT]EEAPSL[CIT]PAPPP |
| 32 | hFibB61-80 | fibrinogen beta | PAPPPISGGGYRARPAKAAA |
| 33 | hFibB226-245 | fibrinogen beta | PCTVSCNIPVVSGKECEEII |
| 34 | hFibB451-470 | fibrinogen beta | QYTWDMAKHGTDDGVVWMNW |
| 35 | hFibB466-485 | Fibrinogen beta | VWMNWKGSWYSMRKMSMKIR |
| 3 | hFibB61-80 | fibrinogen beta | PAPPPISGGGYRARPAKAAA |
| 37 | hFibB151-170 | fibrinogen beta | LKDLWQKRQKQVKDNENVVN |
| 38 | hFibB301-320 | fibrinogen beta | QGFGNVATNTDGKNYCGLPG |
| 39 | COMP 395-414 | Cartilage oligomeric | QKDSDGDGIGDACDNCPQKS |
| | | matrix protein | |
| 40 | Biglycan 238-257 | biglycan | HNKIQAIELEDLLRYSKLYR |
| 41 | Biglycan 247-266 | biglycan | EDLLRYSKLYRLGLGHNQIR |
| 42 | Fibromodulin 345-364 | Fibromodulin | LQVVRLDGNEIKRSAMPADA |
| 43 | Fibromodulin 186-205 | Fibromodulin | NQIS[Cit]VPNNALEGLENLTAL |
| | cit | | |
| 44 | Fibromodulin 332-351 | Fibromodulin | SFCTVVDVVNFSKLQW[Cit]LD |
| | cit | | |
| 45 | Fibromodulin 186-205 | fibromodulin | NQISRVPNNALEGLENLTAL |
| 46 | Fibromodulin186- | Fibromodulin | NQIS[Cit]VPNNALEGLENLTAL |
| | 205cit | | |
| 47 | Fibromodulin 201-220 | fibromodulin | NLTALYLQHDEIQEVGSSMR |
| 48 | Fibromodulin 201-220 | fibromodulin | NLTALYLQHDEIQEVGSSM[Cit] |
| | cit | | |
| 49 | Fibromodulin 216-235 | fibromodulin | GSSMRGLRSLILLDLSYNHL |
| 50 | Fibromodulin 103-122 | fibromodulin | VPS[Cit]MKYVYFQNNQITSIQE |
| | cit | | |
| 51 | Fibromodulin 246-265 | fibromodulin | LEQLYMEHNNVYTVPDSYFR |
| 52 | Lumican 170-189 | Lumican | RLKEDAVSAAFKGLKSLEYL |
| 53 | Lumican 198-217 | Lumican | RLPSGLPVSLLTLYLDNNKI |
| 54 | Clusterin 472-491 | Clusterin | PVEVSRKNPKFMETVAEKAL |
| 55 | Clusterin 221-240 | clusterin | QTHMLDVMQDHFSRASSIID |
| 56 | Clusterin 386-405cit | clusterin | AE[Cit]LT[Cit]KYNELLKSYQWKML |
| 57 | Clusterin 231-250 cit | Clusterin | HFS[Cit]ASSIIDELFQD[Cit]FFT[Cit] |
| 58 | Clusterin 472-491 cit | Clusterin | PVEVS[Cit]KNPKFMETVAEKAL |
| 59 | COMP 453-472 | Cartilageoligomatrix | NSAQEDSDHDGQGDACDDDD |
| | | prot. | |
| 60 | vim166-185 | vimentin | NDKARVEVERDNLAEDIMRL |
| 61 | vim241-260 | vimentin | EIQELQAQIQEQHVQIDVDV |
| 62 | vim391-410 | vimentin | MALDIEIATYRKLLEGEESR |
| 63 | vim421-440 | vimentin | LNLRETNLDSLPLVDTHSKR |
| 64 | vim436-455 | vimentin | THSKRTLLIKTVETRDGQVI |
| 65 | vim16-35cit | vimentin | GGPGTAS[CIT]PSSS[CIT]SYVTTST |
| 66 | vim58-77cit | Vimentin | GGVYAT[CIT]SSAV[CIT]L[CIT]SSVPGV |
| 67 | vim301-320cit | vimentin | AAN[CIT]NNDAL[CIT]QAKQESTEY[CIT] |
| 68 | vim421-440cit | vimentin | LNL[CIT]ETNLDSLPLVDTHSK[CIT] |
| 69 | cfc48-65 | Filaggrin | TIHAHPGSRRGGRHGYHH |
| 70 | cfc48-65 cit2 | Filaggrin | TIHAHPGS[CIT]RGG[CIT]HGYHH |
| 71 | cfc4 | Filaggrin | SHQESTRGRSRG[CIT]SGRSGS |
| 72 | cfc9 | Filaggrin | SHQEST[CIT]GRSRGRSG[CIT]SGS |
| 73 | hFibrinogen cit | Human Fibrinogen | PROTEIN |
| | | Protein | |
| 74 | Osteglycin 176-217 | Osteoglycin | NQLLKLPVLPPKLTLFNAKY |
| 75 | hnRNP-A2 81-99 | hnRNP-A2 | PKRAVAREESGKPGAHVTV |
| 76 | Hsp58 | Hsp58 | VLNRLKVGLQV |
| 77 | PG4 1184-1203 | PG4 | RITEVWGIPSPIDTVFTRCN |
| 78 | Acetyl-calpastatin | Calpastatin | DPMSSTYIEELGKREVTIPPKYRELLA |
| | 184-210 | | |
| 79 | Clusterin 169-187 | Clusterin | QTHMLDVMQDHFSRASSIID |
| 80 | Tenascin C 122-141 | Tenascin | LLSRLEELENLVSSLREQCT |
| 81 | Tenascin C 122-141cit | Tenascin | LLS[CIT]LEELENLVSSL[CIT]EQCT |
| 82 | SerineProtease II | Serine Protease II | VIISINGQSWSANDVSDVI |
| | 433-452 | | |
| 83 | SerineProtease II | Serine Protease II | VVRRGNEDIMITVIPEEIDP |
| | 461-480 | | |
| | hRecombinant | Human rec | PROTEIN |
| | Calpastatin | calpastatin | |
| | Cyclic citrullinated | Filaggrin | CCP1, CCP2, CCP3 are proprietory |
| | peptide | | cocktails of cycl cit peptides for |
| | Cfc1-cyc2 | | commercial ELISA. See Schellekens |
| | | | et al, Arthritis Rheumatism, |
| | | | 43: 155-63, 2000; PMID: 10643712. |
| | CCP cyc Ala-7 | Synthetic Filaggrin | See Schellekens et al, J. Clinical |
| | | derivative | Investigation, 101: 273-81, 1998; |
| | | | PMID: 9421490. |
| | CCP cyc Ala-6 | Synthetic Filaggrin | See Schellekens et al, J. Clinical |
| | | derivative | Investigation, 101: 273-81, 1998; |
| | | | PMID: 9421490. |
| | CCP cyc 0112-15 | Synthetic Filaggrin | See Schellekens et al, J. Clinical |
| | | derivative | Investigation, 101: 273-81, 1998; |
| | | | PMID: 9421490. |
| | Aggrecan protein | Aggrecan | large cartilage proteoglycan |
| | Decorin protein | Decorin | Small cartilage proteoglycan |
| | hnRNP D protein | hnRNP D | |
| | hnRNP A2/B1 | hnRNP A2/B1 | |
| | BiP endoplasmic | BiP | |
| | reticulum chaperone | | |
| | 78 protein | | |
| | HSP70 protein | Heat shock protein | |
| | | 70 | |

Biglycan is a small cellular or pericellular matrix proteoglycan that is closely related in structure to two other small proteoglycans, decorin and fibromodulin. This protein is thought to function in connective tissue metabolism by binding to collagen fibrils and transforming growth factor-beta. The genetic sequence of human biglycan may be accessed at Genbank, accession number NM_001711.

Calpastatin is an endogenous calpain (calcium-dependent cysteine protease) inhibitor. It consists of an N-terminal domain L and four repetitive calpain-inhibition domains (domains 1-4), and it is involved in the proteolysis of amyloid precursor protein. The calpain/calpastatin system is involved in numerous membrane fusion events, such as neural vesicle exocytosis and platelet and red-cell aggregation. The encoded protein is also thought to affect the expression levels of genes encoding structural or regulatory proteins. The genetic sequence of human calpastatin may be accessed at Genbank, accession number NM_001750.

Clusterin, or sulfated glycoprotein-2 (SGP-2) is a normal constituent of human blood. It consists of two 40-kD chains, alpha and beta, covalently joined by disulfide bonds. It is a member of the human complement system, and also called complement lysis inhibitor. It acts as a control mechanism of the complement cascade; specifically, it prevents the binding of a C5b-C7 complex to the membrane of the target cell and in this way inhibits complement-mediated cytolysis. The genetic sequence of human clusterin may be accessed at Genbank, accession number NM_001831.

The alpha-1 chain of type II collagen is a fibrillar collagen found in cartilage and the vitreous humor of the eye. There are two transcripts identified for this gene. The genetic sequence of human type II collagen may be accessed at Genbank, accession number NM_001844.

Cartilage oligomeric matrix protein is a noncollagenous extracellular matrix (ECM) protein. It consists of five identical glycoprotein subunits, each with EGF-like and calcium-binding (thrombospondin-like) domains. Oligomerization results from formation of a five-stranded coiled coil and disulfides. The genetic sequence of human COMP may be accessed at Genbank, accession number NM_000095.

Fibrinogen is a blood-borne glycoprotein comprised of three pairs of nonidentical polypeptide chains. Following vascular injury, fibrinogen is cleaved by thrombin to form fibrin which is the most abundant component of blood clots. In addition, various cleavage products of fibrinogen and fibrin regulate cell adhesion and spreading, display vasoconstrictor and chemotactic activities, and are mitogens for several cell types. The genetic sequence of human fibrinogen beta chain may be accessed at Genbank, accession number NM_000508. The genetic sequence of human fibrinogen beta chain may be accessed at Genbank, accession number NM_005141. The genetic sequence of human fibrinogen gamma chain may be accessed at Genbank, accession number NM_000509.

Fibromodulin is a member of a family of small interstitial proteoglycans, containing a central region composed of leucine-rich repeats with 4 keratan sulfate chains flanked by disulfide-bonded terminal domains. It may participate in the assembly of the extracellular matrix as it interacts with type I and type II collagen fibrils and inhibits fibrillogenesis in vitro. It may also regulate TGF-beta activities by sequestering TGF-beta into the extracellular matrix. The genetic sequence of human fibromodulin may be accessed at Genbank, accession number NM_002023.

Histones are basic nuclear proteins that are responsible for the nucleosome structure of the chromosomal fiber in eukaryotes. Nucleosomes consist of approximately 146 bp of DNA wrapped around a histone octamer composed of pairs of each of the four core histones (H2A, H2B, H3, and H4). The chromatin fiber is further compacted through the interaction of a linker histone, H1, with the DNA between the nucleosomes to form higher order chromatin structures. This gene is intronless and encodes a member of the histone H2A family. Transcripts from this gene contain a palindromic termination element. The genetic sequence of human histone H2A may be accessed at Genbank, accession number NM_170745. The genetic sequence of human histone H2B may be accessed at Genbank, accession number NG_000009.

The genetic sequence of human heat shock protein HSP90, beta chain may be accessed at Genbank, AY956763. The genetic sequence of human heat shock protein HSP90, alpha chain may be accessed at Genbank, NM_001017963.

The genetic sequence of human cartilage glycoprotein-39, (gp 39) may be accessed at Genbank, NM_001276.

Lumican is a member of the small leucine-rich proteoglycan (SLRP) family that includes decorin, biglycan, fibromodulin, keratocan, epiphycan, and osteoglycin. In these bifunctional molecules, the protein moiety binds collagen fibrils and the highly charged hydrophilic glycosaminoglycans regulate interfibrillar spacings. Lumican is the major keratan sulfate proteoglycan of the cornea but is also distributed in interstitial collagenous matrices throughout the body. Lumican may regulate collagen fibril organization and circumferential growth, corneal transparency, and epithelial cell migration and tissue repair. NM_002345.

Antigens for evaluation of demyelinating diseases may comprise epitopes from proteolipid protein (PLP); myelin basic protein (MBP); myelin oligodendrocyte protein (MOG); cyclic nucleotide phosphodiesterase (CNPase); myelin-associated glycoprotein (MAG), and myelin-associated oligodendrocytic basic protein (MBOP); alpha-B-crystalin (a heat shock protein); viral and bacterial mimicry peptides, e.g. influenza, herpes viruses, hepatitis B virus, etc.; OSP (oligodendrocyte specific-protein); citrulline-modified MBP (the C8 isoform of MBP in which 6 arginines have been de-imminated to citrulline), etc. The integral membrane protein PLP is a dominant autoantigen of myelin. At least 26 MBP epitopes have been reported (Meinl et al. (1993) J. Clin. Invest. 92:2633-2643). Notable are residues 1-11, 59-76 and 87-99. Immunodominant MOG epitopes that have been identified in several mouse strains include residues 1-22, 35-55, 64-96.

Antigens for evaluation of insulin dependent diabetes mellitus may comprise the antigens and epitopes derived from IA-2; IA-2beta; GAD; insulin; preproinsulin; HSP; glima 38; ICA69; p52; and other proteins present in the beta cells of the pancreas and pancreatic islets.

Antigens for evaluation of systemic lupus erythematosus (SLE) may include DNA; phospholipids; nuclear antigens; Ro; La; U1 ribonucleoprotein; Ro60 (SS-A); Ro52 (SS-A); La (SS-B); calreticulin; Grp78; Scl-70; histone; Sm protein; and chromatin, etc.

Antigens for evaluation of autoimmune uveitis may include S-antigen, and interphotoreceptor retinoid binding protein (IRBP), etc.

Antigens for evaluation of myasthenia gravis may include epitopes with the acetylcholine receptor. For Grave's disease epitopes may include the Na+/I− symporter; thyrotropin receptor; Tg; and TPO. Sjogren's syndrome panels may include SSA (Ro); SSB (La); and fodrin. Panels for pemphigus vulgaris may include desmoglein-3. Panels for myositis may include tRNA synthetases (e.g., threonyl, histidyl, alanyl, isoleucyl, and glycyl); Ku; PM/Scl; SSA; U1 sn-ribonuclear protein; Mi-1; Mi-1; Jo-1; Ku; and SRP. Panels for scleroderma may include Scl-70; centromere proteins; U1 ribonuclear proteins; and fibrillarin. Panels for primary biliary cirrhosis may include pyruvate dehydrogenase E2 and alpha-ketoglutarate dehydrogenase components. Panels for pernicious anemia may include intrinsic factor; and glycoprotein beta subunit of gastric H/K ATPase.

Antigens for evaluation of psoriasis include cytokeratin 17, and other keratins and collagens. Although psoriasis is considered an autoimmune disease, increasing evidence suggests an important role for bacteria in its initiation and/or propagation. Colonization and infection with Staphylococcus and Streptococcus have been reported to exacerbate psoriasis. Antigens may include bacterial and viral antigens, e.g. antigens derived from Staphylococcus or Streptococcus, other physiologic or pathologic bacterial skin flora, papilloma virus type 5, and the like.

### Cytokines

Cytokines are messenger molecules produced by B cells, T cells, macrophage, dendritic cells and other immune and host cells. Cytokines play roles in the pathogenesis of rheumatoid arthritis, multiple sclerosis and other autoimmune diseases. Cytokines include chemokines, lymphokines, growth factors, angiogenesis factors, and other secreted and cell surface molecules that transmit signals to other cells. Cytokines include, but are not limited to the molecules listed in Table 2.

TABLE 2
Cytokine Names

| | | |
|---|---|---|
| TNFa | IL-13 | MCP-1 |
| IL-1a | IL-15 | MCP-2 |
| IL-1b | IFNγ | MCP-3 |
| IL-2 | IL-17 | Rantes |
| IL-3 | IL-18 | IL-18 |
| IL-4 | IL-23 | IP-10 |
| IL-5 | Osteopontin | MIP-1a |
| IL-6 | TGFb | MIP-1b |
| IL-7 | VEGF | MIP-2 |
| IL-8 | IGF | MIP-3a |
| IL-10 | G-CSF | MIP-5 |
| IL-12p40 | GM-CSF | Eotaxin |
| IL-12p70 | PDGF | Rantes |
| IL-11 | Leptin | Flt-3 |

### Conditions for Analysis and Therapy

The compositions and methods of the invention find use in combination with a variety of autoimmune conditions, which include, without limiting, the following conditions.

Rheumatoid Arthritis is a chronic syndrome characterized by usually symmetric inflammation of the peripheral joints, potentially resulting in progressive destruction of articular and periarticular structures, with or without generalized manifestations. The cause is unknown. A genetic predisposition has been identified and, in white populations, localized to a pentapeptide in the HLA-DR beta1 locus of class II histocompatibility genes. Environmental factors may also play a role. Immunologic changes may be initiated by multiple factors. About 0.6% of all populations are affected, women two to three times more often than men. Onset may be at any age, most often between 25 and 50 yr.

Prominent immunologic abnormalities that may be important in pathogenesis include immune complexes found in joint fluid cells and in vasculitis. Plasma cells produce antibodies that contribute to these complexes. Lymphocytes that infiltrate the synovial tissue are primarily T helper cells, which can produce pro-inflammatory cytokines. Macrophages and their cytokines (e.g., tumor necrosis factor, granulocyte-macrophage colony-stimulating factor) are also abundant in diseased synovium. Increased adhesion molecules contribute to inflammatory cell emigration and retention in the synovial tissue. Increased macrophage-derived lining cells are prominent along with some lymphocytes and vascular changes in early disease.

In chronically affected joints, the normally delicate synovium develops many villous folds and thickens because of increased numbers and size of synovial lining cells and colonization by lymphocytes and plasma cells. The lining cells produce various materials, including collagenase and stromelysin, which can contribute to cartilage destruction; interleukin-1, which stimulates lymphocyte proliferation; and prostaglandins. The infiltrating cells, initially perivenular but later forming lymphoid follicles with germinal centers, synthesize interleukin-2, other cytokines, RF, and other immunoglobulins. Fibrin deposition, fibrosis, and necrosis also are present. Hyperplastic synovial tissue (pannus) may erode cartilage, subchondral bone, articular capsule, and ligaments. PMNs are not prominent in the synovium but often predominate in the synovial fluid.

Onset is usually insidious, with progressive joint involvement, but may be abrupt, with simultaneous inflammation in multiple joints. Tenderness in nearly all inflamed joints is the most sensitive physical finding. Synovial thickening, the most specific physical finding, eventually occurs in most involved joints. Symmetric involvement of small hand joints (especially proximal interphalangeal and metacarpophalangeal), foot joints (metatarsophalangeal), wrists, elbows, and ankles is typical, but initial manifestations may occur in any joint.

Psoriasis is a chronic skin disease, characterized by scaling and inflammation. Psoriasis affects 1.5 to 2 percent of the United States population, or almost 5 million people. It occurs in all age groups and about equally in men and women. People with psoriasis suffer discomfort, restricted motion of joints, and emotional distress. When psoriasis develops, patches of skin thicken, redden, and become covered with silvery scales, referred to as plaques. Psoriasis most often occurs on the elbows, knees, scalp, lower back, face, palms, and soles of the feet. The disease also may affect the fingernails, toenails, and the soft tissues inside the mouth and genitalia. About 10 percent of people with psoriasis have joint inflammation that produces symptoms of arthritis.

When skin is wounded, a wound healing program is triggered, also known as regenerative maturation. Lesional psoriasis is characterized by cell growth in this alternate growth program. In many ways, psoriatic skin is similar to skin healing from a wound or reacting to a stimulus such as infection, where the keratinocytes switch from the normal growth program to regenerative maturation. Cells are created and pushed to the surface in as little as 2-4 days, and the skin cannot shed the cells fast enough. The excessive skin cells build up and form elevated, scaly lesions. The white scale (called “plaque”) that usually covers the lesion is composed of dead skin cells, and the redness of the lesion is caused by increased blood supply to the area of rapidly dividing skin cells.

The exact cause of psoriasis in humans is not known, although it is generally accepted that it has a genetic component, and a recent study has established that it has an autoimmune component. Whether a person actually develops psoriasis is hypothesized to depend on something “triggering” its appearance. Examples of potential “trigger factors” include systemic infections, injury to the skin (the Koebner phenomenon), vaccinations, certain medications, and intramuscular injections or oral steroid medications. The chronic skin inflammation of psoriasis is associated with hyperplastic epidermal keratinocytes and infiltrating mononuclear cells, including CD4+ memory T cells, neutrophils and macrophages.

SLE. Systemic lupus erythematosus (SLE) is an autoimmune disease characterized by polyclonal B cell activation, which results in a variety of anti-protein and non-protein autoantibodies (see Kotzin et al. (1996) Cell 85:303-306 for a review of the disease). These autoantibodies form immune complexes that deposit in multiple organ systems, causing tissue damage. SLE is a difficult disease to study, having a variable disease course characterized by exacerbations and remissions. For example, some patients may demonstrate predominantly skin rash and joint pain, show spontaneous remissions, and require little medication. The other end of the spectrum includes patients who demonstrate severe and progressive kidney involvement (glomerulonephritis) that requires therapy with high doses of steroids and cytotoxic drugs such as cyclophosphamide.

Multiple factors may contribute to the development of SLE. Several genetic loci may contribute to susceptibility, including the histocompatibility antigens HLA-DR2 and HLA-DR3. The polygenic nature of this genetic predisposition, as well as the contribution of environmental factors, is suggested by a moderate concordance rate for identical twins, of between 25 and 60%.

Many causes have been suggested for the origin of autoantibody production. Proposed mechanisms of T cell help for anti-dsDNA antibody secretion include T cell recognition of DNA-associated protein antigens such as histones and recognition of anti-DNA antibody-derived peptides in the context of class II MHC. The class of antibody may also play a factor. In the hereditary lupus of NZB/NZW mice, cationic IgG2a anti-double-stranded (ds) DNA antibodies are pathogenic. The transition of autoantibody secretion from IgM to IgG in these animals occurs at the age of about six months, and T cells may play an important role in regulating the IgG production.

Disease manifestations result from recurrent vascular injury due to immune complex deposition, leukothrombosis, or thrombosis. Additionally, cytotoxic antibodies can mediate autoimmune hemolytic anemia and thrombocytopenia, while antibodies to specific cellular antigens can disrupt cellular function. An example of the latter is the association between anti-neuronal antibodies and neuropsychiatric SLE.

Autoimmune diseases also include a number of demyelinating diseases, which may be characterized according to the presence of autoantibodies specific for lipids and lipoproteins associated with the nervous system, and in particular with myelin. Autoantibodies directed against non-myelin (axonal, interstitial) and ubiquitous proteins such as heat shock proteins may occur and may also play a role. Myelin sheaths, which cover many nerve fibers, are composed of lipoprotein layers formed in early life. Myelin formed by the oligodendroglia in the CNS differs chemically and immunologically from that formed by the Schwann cells peripherally, but both types have the same function: to promote transmission of a neural impulse along an axon. Demyelinating diseases include those that affect the central nervous system, and those that affect the peripheral nervous system. CNS conditions include multiple sclerosis, and the animal model experimental autoimmune encephalomyelitis (EAE), which are slowly progressive CNS diseases characterized by disseminated patches of demyelination in the brain and spinal cord, resulting in multiple and varied neurologic symptoms and signs, usually with remissions and exacerbations.

Plaques of demyelination, with destruction of oligodendroglia and perivascular inflammation, are disseminated throughout the CNS, primarily in the white matter, with a predilection for the lateral and posterior columns (especially in the cervical and dorsal regions), the optic nerves, and periventricular areas. Tracts in the midbrain, pons, and cerebellum are also affected as is gray matter in the cerebrum and spinal cord. Cell bodies and axons are usually preserved, especially in recent lesions. Later, axons may be destroyed, especially in the long tracts, and a fibrous gliosis makes the tracts appear sclerotic. Recent and old lesions may coexist. Chemical changes in lipid and protein constituents of myelin occur in and around the plaques.

MS is characterized by various symptoms and signs of CNS dysfunction, with remissions and recurring exacerbations. The most common presenting symptoms are paresthesias in one or more extremities, in the trunk, or on one side of the face; weakness or clumsiness of a leg or hand; or visual disturbances, e.g. partial blindness and pain in one eye (retrobulbar optic neuritis), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances; all indicate scattered CNS involvement and often occur months or years before the disease is recognized. Excess heat may accentuate symptoms and signs.

The course is highly varied, unpredictable, and, in most patients, remittent. At first, months or years of remission may separate episodes, especially when the disease begins with retrobulbar optic neuritis. However, some patients have frequent attacks and are rapidly incapacitated; for a few the course can be rapidly progressive.

Diagnosis is indirect, by deduction from clinical and laboratory features. Typical cases can usually be diagnosed confidently on clinical grounds. The diagnosis can be suspected after a first attack. Later, a history of remissions and exacerbations and clinical evidence of CNS lesions disseminated in more than one area are highly suggestive.

MRI, the most sensitive diagnostic imaging technique, may show plaques. It may also detect treatable nondemyelinating lesions at the junction of the spinal cord and medulla (eg, subarachnoid cyst, foramen magnum tumors) that occasionally cause a variable and fluctuating spectrum of motor and sensory symptoms, mimicking MS. Gadolinium-contrast enhancement can distinguish areas of active inflammation from older brain plaques. MS lesions may also be visible on contrast-enhanced CT scans; sensitivity may be increased by giving twice the iodine dose and delaying scanning (double-dose delayed CT scan).

Acute disseminated encephalomyelitis (ADEM) is characterized by a brief but intense attack of the CNS and results in demyelination. It often follows viral infections and vaccinations, such as for measles, mumps, or rubella, and more frequently affects children than adults. ADEM is characterized by acute onset of symptoms that can include encephalitis-like symptoms such as fever, fatigue, headache, nausea and vomiting. It may also cause visual loss (optic neuritis) in one or both eyes, and weakness and paralysis. ADEM is sometimes misdiagnosed as a severe initial attack of multiple sclerosis. In contrast to MS, ADEM usually consists of a single episode or attack.

Peripheral neuropathies include Guillain-Barre syndrome (GBS) with its subtypes acute inflammatory demyelinating polyradiculoneuropathy, acute motor axonal neuropathy, acute motor and sensory axonal neuropathy, Miller Fisher syndrome, and acute pandysautonomia; chronic inflammatory demyelinating polyneuropathy (CIDP) with its subtypes classical CIDP, CIDP with diabetes, CIDP/monoclonal gammopathy of undetermined significance (MGUS), sensory CIDP, multifocal motor neuropathy (MMN), multifocal acquired demyelinating sensory and motor neuropathy or Lewis-Sumner syndrome, multifocal acquired sensory and motor neuropathy, and distal acquired demyelinating sensory neuropathy; IgM monoclonal gammopathies with its subtypes Waldenstrom's macroglobulinemia, myelin-associated glycoprotein-associated gammopathy, polyneuropathy, organomegaly, endocrinopathy, M-protein, skin changes syndrome, mixed cryoglobulinemia, gait ataxia, late-onset polyneuropathy syndrome, and MGUS.

Diabetes Mellitus (DM) is a syndrome characterized by hyperglycemia resulting from absolute or relative impairment in insulin secretion and/or insulin action. Although it may occur at any age, type I diabetes mellitus (T1 D) most commonly develops in childhood or adolescence and is the predominant type of DM diagnosed before age 30. This type of diabetes accounts for 10 to 15% of all cases of DM and is characterized clinically by hyperglycemia and a propensity to DKA. The pancreas produces little or no insulin.

About 80% of patients with T1D have specific HLA phenotypes associated with detectable serum islet cell cytoplasmic antibodies and islet cell surface antibodies (antibodies to glutamic acid decarboxylase (GAD) and to insulin are found in a similar proportion of cases). In these patients, T1 D results from a genetically susceptible, immune-mediated, selective destruction of >90% of their insulin-secreting beta cells. Their pancreatic islets exhibit insulitis, which is characterized by an infiltration of T lymphocytes accompanied by macrophages and B lymphocytes and by the loss of most of the beta cells, without involvement of the glucagon-secreting alpha cells. Cell-mediated immune mechanisms are believed to play the major role in the beta-cell destruction. The antibodies present at diagnosis usually become undetectable after a few years. They may be primarily a response to beta-cell destruction, but some are cytotoxic for beta cells and may contribute to their loss. The clinical onset of T1D may occur in some patients years after the insidious onset of the underlying autoimmune process.

In white populations, a strong association exists between T1 D diagnosed before age 30 and specific HLA-D phenotypes (HLA-DR3, HLA-DR4, and HLA-DR3/HLA-DR4). One or more genes that convey susceptibility to T1 D are believed to be located near or in the HLA-D locus on chromosome 6. Specific HLA-DQ alleles appear to be more intimately related to risks for or protection from T1D than HLA-D antigens, and evidence suggests that genetic susceptibility to type T1D is probably polygenic. Only 10 to 12% of newly diagnosed children with T1D have a first-degree relative with T1 D, and the concordance rate for T1D in monozygotic twins is <=50%. Thus, in addition to the genetic background, environmental factors affect the appearance of T1 D. Such environmental factors may be viruses (congenital rubella, mumps, and coxsackie B viruses may incite the development of autoimmune beta-cell destruction) and exposure to cow's milk rather than maternal milk in infancy (a specific sequence of albumin from cow's milk may cross-react with islet protein). Geography may play a role in exposure, as the incidence of T1D is particularly high in Finnland and Sardinia.

### Therapeutic Agents

General classes of drugs commonly used in the non-antigen specific treatment of autoimmune disease include corticosteroids and disease modifying drugs. Corticosteroids have a short onset of action, but many disease modifying drugs take several weeks or months to demonstrate a clinical effect. These agents include methotrexate, leflunomide (Arava™) etanercept (Enbrel™), infliximab (Remicade™), adalimumab (Humira™), anakinra (Kineret™) rituximab (Rituxan™), CTLA4-Ig (abatacept), antimalarials, gold salts, sulfasalazine, d-penicillamine, cyclosporin A, cyclophosphamide azathioprine; and the like.

Corticosteroids, e.g. prednisone, methylpredisone, prednisolone, solumedrol, etc. have both anti-inflammatory and immunoregulatory activity. They can be given systemically or can be injected locally. Corticosteroids are useful in early disease as temporary adjunctive therapy while waiting for disease modifying agents to exert their effects. Corticosteroids are also useful as chronic adjunctive therapy in patients with severe disease.

Disease modifying anti-rheumatoid drugs, or DMARDs have been shown to alter the disease course and improve radiographic outcomes in RA. It will be understood by those of skill in the art that these drugs are also used in the treatment of other autoimmune diseases.

Methotrexate (MTX) is a frequent first-line agent because of its early onset of action (4-6 weeks), good efficacy, favorable toxicity profile, ease of administration, and low cost. MTX is the only conventional DMARD agent in which the majority of patients continue on therapy after 5 years. MTX is effective in reducing the signs and symptoms of RA, as well as slowing or halting radiographic damage. Although the immunosuppressive and cytotoxic effects of MTX are in part due to the inhibition of dihydrofolate reductase, the anti-inflammatory effects in rheumatoid arthritis appear to be related at least in part to interruption of adenosine and TNF pathways. The onset of action is 4 to 6 weeks, with 70% of patients having some response. A trial of 3 to 6 months is suggested.

Antimalarials such as hydroxychloroquine and chloroquine are rapidly absorbed, relatively safe, well-tolerated and often effective remittive agents for the treatment of rheumatoid arthritis, particularly mild to moderate disease. Hydroxychloroquine (Plaquenil, 200 mg tablets) is the drug of choice among antimalarials. The usual dose is 400 mg/day (6 mg/kg) but 600 mg/day is sometimes used. Normally it is prescribed as a single nighttime dose to avoid gastrointestinal symptoms. A period of 2 to 4 months is usual to take effect. A 6-month period without clinical effect should be considered a drug failure.

Sulfasalazine (SSZ) is another effective DMARD for the treatment of RA. Its mechanism of action in RA is unknown. Like the other DMARDs, it has been shown not only to reduce the signs and symptoms of RA but also to slow or halt radiographic progression. It can cause hypersensitivity reactions due to sulfa allergy, mild gastrointestinal, and occasionally, mild cytopenias. The usual dose is 2-3 grams per day in a twice daily dosing regimen. Blood monitoring is every 1-3 months depending on dose. Sulfasalazine is a good alternative to methotrexate for patients with liver disease.

A 56-week Combination Therapy in Rheumatoid Arthritis (COBRA) trial demonstrated that step-down combination therapy with prednisolone, methotrexate, and sulfasalazine (SSZ) was superior to SSZ monotherapy for suppressing disease activity and progression of rheumatoid arthritis (RA). (COBRA: Arthritis Rheum, 2002 February; 46(2):347-56). In a follow up study, the authors investigated whether the benefits of COBRA therapy were sustained over time, while the treating rheumatologists were not required to adhere to a pre-specified treatment protocol. Outcomes were analyzed on the basis of intent-to-treat, starting with data obtained at the last visit of the COBRA trial (56 weeks after baseline). After adjustment for differences in treatment and disease activity during follow-up, the differences between combination therapy-treated and control groups in regard to the rate of progression was statistically significant for each single year of follow up (4-5 years). The disability (based on the Health Assessment Questionnaire, HAQ) score did not change significantly over time. Independent baseline predictors of radiological progression over time (apart from treatment allocation) were rheumatoid factor positivity, radiographic scores (Sharp scores), and disease activity score (DAS28). The authors conclude that an initial 6-month cycle of intensive combination treatment that included high-dose corticosteroids resulted in sustained suppression of the rate of radiologic progression in patients with early RA, independent of subsequent anti-rheumatic therapy. The impressive results of this study suggest that aggressive combination therapy very early in the course of RA provides long-term benefit, even though the treatment course lasted only 6 months. However, the question of the role of newer biologicals such as TNF blockers and other targeted therapies in early RA (CTLA4Ig, IL-6R, etc) is not addressed by this trial. Additionally, acceptance of the complex COBRA medication is relatively low with both prescribing rheumatologists and RA patients in the Netherlands (Ann Rheum Dis. 2007 Mar. 28), underscoring the need for better biomarkers to predict response to individual drugs with greater potency but also the potential to cause serious side effects.

Also of concern is the potential for overtreatment of the subset of early arthritis patients who will experience a benign disease course. It is well established that a subset of early arthritis patients, including patients with early RA, will experience spontaneous natural remission in the absence of therapeutic intervention. Thus, biomarkers are needed to identify and differentiate such patients from patients who will develop full-blow and/or severe RA. Patients predicted to have benign and naturally remitting RA would likely be treated with NSAIDs and other “low-impact” therapies, while patients predicted to evolve to established RA would be treated more aggressively with DMARD therapy, and patients predicted to develop severe debilitating RA would be treated most aggressively with highly potent DMARD therapy. Such a therapeutic strategy could both reduce the incidence of RA, by reducing the number of patients that progress from early arthritis or RA to established RA, as well as reduce the mortality and morbidity from RA.

Leflunomide (ARAVA™) was approved by the FDA and became available as a new DMARD agent for rheumatoid arthritis in October 1998. In clinical trials, its efficacy was similar to that of methotrexate and it represents a viable alternative to patients who have failed or are intolerant to methotrexate. Leflunomide has been demonstrated to slow radiographic progression and damage in RA. It can also be combined with methotrexate in patients with no preexisting liver disease, as long as the liver function tests are carefully monitored. The mechanism of action of leflunomide is not fully understood but may be related to its ability to inhibit tyrosine kinase activity and inhibit de novo pyrimidine biosynthesis through the inhibition of the enzyme dihydroorotate dehydrogenase. In vitro studies have demonstrated the inhibition of mitogen and IL-2 stimulated T cells. To achieve steady state, a loading dose of 100 mg daily for three days can be given followed by 20 mg daily. However, more recent recommendations are for a starting dose of 20 mg daily. The dose may be reduced to 10 mg daily if not tolerated or in patients having difficulty metabolizing or excreting the drug. Onset of action is in 4-8 weeks.

Tumor necrosis factor alpha (TNF-α, also referred to as TNF) is a pro-inflammatory cytokine produced by macrophages and lymphocytes. It is found in large quantities in the rheumatoid joint and is produced locally in the joint by synovial macrophages and lymphocytes infiltrating the joint synovium. Extensive clinical trial data have confirmed the efficacy of all three currently available TNF inhibitors in relieving the signs and symptoms of RA, and in slowing or halting radiographic damage. The strategies for inhibiting TNF that have been most extensively studied to date consist of monoclonal anti-TNF antibodies and soluble TNF receptors (sTNF-R). Both constructs will bind to circulating TNF-α, thus limiting its ability to engage cell membrane-bound TNF receptors and activate inflammatory pathways. Soluble TNF-R, but not anti-TNF antibodies, also bind lymphotoxin.

One of the monoclonal anti-TNF antibodies is infliximab (REMICADE®), originally referred to as cA2. Infliximab is a chimeric human/mouse monoclonal anti-TNFα antibody composed of the constant regions of human (Hu) IgG1κ, coupled to the Fv region of a high-affinity neutralizing murine anti-huTNFa antibody. The antibody exhibits high affinity (Ka 1010/mol) for recombinant and natural huTNFα, and neutralizes TNF-mediated cytotoxicity and other functions in vitro. An alternate strategy has been to develop a fully human anti-TNF monoclonal antibody. One such antibody, known as D2E7, also known as adalumimab (HUMIRA™), was generated by phage display technology. A high affinity murine anti-TNF monoclonal antibody was used as a template for guided selection, which involves complete replacement of the murine heavy and light chains with human counterparts and subsequent optimization of the antigen-binding affinity. D2E7 (adalimumab, HUMIRA™) received FDA approval in December, 2002.

Alternatively, soluble TNF-R have been engineered as fusion proteins in which the extracellular ligand-binding portion of the huTNF-RI or huTNF-RII is coupled to a human immunoglobulin-like molecule. Although TNF-RI is thought to mediate most of the biological effects of TNF in vivo, engineered sTNF-RI and sTNF-RII constructs both appear to be effective in vivo inhibitors of TNF. Etanercept (sTNF-RII:Fc; ENBREL™) is the best studied of the sTNF-R and is approved for the treatment of rheumatoid arthritis in adults and in children. It is a dimeric construct in which two sTNF-RII (p75) are linked to the Fc portion of human IgG1. The dimeric receptor has a significantly higher affinity for TNF-α than the monomeric receptor (50-1000-fold higher), and the linkage to the Fc structure significantly prolongs the half-life of the construct in vivo. Although it also has an unnatural linkage site, anti-etanercept antibodies have been infrequent. Another mechanism for prolonging the half-life of monomeric receptors is via conjugation with polyethylene glycol. One such construct, PEG-sTNF-RI (p55), has shown efficacy in several animal models of arthritis and is now in early clinical trials.

It is well established that only approximately ⅓ of patients exhibit a robust clinical response following initiation of any one of the 3 FDA-approved anti-TNF therapies (etanercept, adalimumab and remicade). As described below, clinical response is measured based on the American College of Rheumatology (ACR) response criteria (detailed below), and the ⅓ of patients that are experience robust clinical responses experience an ACR50 or greater response. A second ⅓ of patients experience a partial response to any one of the FDA approved agents, approximately an ACR20 response. The remaining ⅓ of RA patients exhibit no meaningful clinical response when initiated on an approved anti-TNF therapy. There is great clinical need for biomarkers to identify RA patients likely to respond vs. not respond to an ant-TNF agent given: (1) the potentially serious side effects of anti-TNF agents including (a) activation of tuberculosis, (b) increased rates of serious and life threatening infections, and (c) increased rates of demyelinating lesions; (2) the significant expense of anti-TNF therapies (approximately $15,000 USD per year of therapy), and (3) the availability of multiple other potential effective small molecule and biological agents (methotrexate, leuflonamide, anakinra, CTLAr-Ig).

Studies of early rheumatoid arthritis can establish which drugs or combinations of drugs perform best to delay or prevent irreversible damage (see COBRA study above). An ongoing study, The BeST study, focuses on different combinations of established DMARDs in conjunction with the TNF blocker infliximab (BeSt Study: Arthritis Rheum. 2005 November; 52(11):3381-90). This study aimed at comparing the efficiency of four treatment approaches to minimize disease progression in patients with early RA. Patients with active rheumatoid arthritis having symptoms of less than 2 years duration were randomized to one of four treatment arms: (1) Sequential monotherapy starting with methotrexate (MTX), then sulphasalazine (SSA), then leflunomide, then MTX with infliximab (IFX) (group 1, n=126); (2) Step-up combination therapy starting with MTX, then adding SSA, then hydroxychloroquine and then prednisone, then switching to MTX with IFX (group 2, n=121); (3) Initial combination therapy with MTX, SSA and a tapered high dose prednisone, then MTX with cyclosporin A and prednisone, then MTX with IFX (group 3, n=133); (3) Initial combination therapy with MTX and IFX, next leflunomide, then SSA, then MTX with cyclosporin A and prednisone (group 4, n=128). Better radiographic scores were observed in the more aggressive treatment arms (groups 3 and 4), supporting the call for early aggressive therapy. An important finding from the study is that similar clinical outcomes were achieved in all treatment groups when patients were followed by Disease Active Score (DAS) scoring and therapy was changed based on a protocol established before the trial had started. As underscored by previous clinical studies, rheumatologists need to quantify disease activity in response to therapy, regardless of which therapy is chosen. Additional clinical trials in early RA involve a number of the novel biological DMARDs including MTX, anti-TNF agents, and CTLA4-Ig both as individual therapies as well as in combination (e.g. MTX; MTX+anti-TNF; anti-TNF; MTX+CTLA4-Ig; CTLA4-Ig).

The ability to monitor disease progression and response to therapy as provided by the methods of the invention provides a critical addition to the clinical armentarium of physicians for improved outcome measurement.

Soluble Interleukin-1 (IL-1) Receptor therapy. IL-1 is a cytokine that has immune and pro-inflammatory actions and has the ability to regulate its own expression by autoinduction. Evidence supports the fact that the level of disease activity in RA, and progression of joint destruction, correlate with plasma and synovial fluid levels of IL-1. IL-1ra is an endogenous receptor antagonist. Evidence supporting the anti-inflammatory role of IL-1ra in vivo is demonstrated by the observation that IL-1ra deficient mice spontaneously develop autoimmune diseases similar to rheumatoid arthritis and arteriitis.

Anakinra (KINERET™) is a human recombinant IL-1 receptor antagonist (hu rIL-1ra) approved by the FDA for the treatment of RA. Anakinra can be used alone or in combination with DMARDs other than TNF blocking agents (Etanercept, Infliximab). Anakinra is a recombinant, nonglycosylated form of the human IL-1ra. It differs from the native nonglycosylated IL-1ra by the addition of an N-terminal methionine. Anakinra blocks the biologic activity of IL-1 by binding to IL-1R type I with the same affinity as IL-1β. Usual time to effect is 2 to 4 weeks.

Cytotoxic T lymphocyte-associated antigen 4 (CTLA4) is an immunoregulatory protein expressed on the T cell surface after activation. It binds to CD80 or CD86, blocks their interaction with CD28, and thus acts as an off-switch for cell activation. CTLA4Ig is a genetically engineered fusion protein that consists of a human CTLA4 portion fused to a constant IgG1 region (also know as Abetacept, produced by Bristol-Myers Squib, New York City, N.Y., USA). This molecule binds to CD80 and CD86 and thereby inhibits T cell co-stimulation. Abetacept was approved by the US Food and Drug Administration for the treatment of RA. Only a minority of patients who had failed anti-TNF therapy exhibited significant clinical improvement in response to CTLA4-Ig therapy. Subsets of RA patients can be classified as responders and non-responders to therapy with CTLA4-Ig, and responsiveness is determined by the underlying etiology of an individual patient's disease. Identification of autoantibody and cytokine biomarkers identifies molecular subytpes of RA that are responsive to agents such as CTLA4-Ig or anti-TNF.

Rituximab. The CD20 antigen is present on the cell surface of all pre-plasma cell stages of B cell differentiation. The mature plasma cell loses the CD20 antigen, and thus it serves as a relatively specific marker for B cells. Rituximab (Roche Pharmaceuticals, Basel, Switzerland; Genentech, South San Francisco, USA; IDEC Pharmaceuticals, San Diego, USA), a genetically engineered human-mouse chimeric monoclonal antibody against the CD20 antigen, binds to the CD20 antigen on the B cell surface and efficiently depletes B cells by antibody-dependent and complement-dependent cell lysis. Therapeutic monoclonal antibodies directed against other B cell surface antigens such as CD19, CD21 and CD22 are currently under development. A minority of patient who failed anti-TNF therapy exhibited an ACR50 or greater response to rituximab therapy. Subsets of RA patients can be classified as responders and non-responders to therapy with anti-B cell therapies, and responsiveness is determined by the underlying etiology of an individual patient's disease. Identification of autoantibody and cytokine biomarkers identifies molecular subytpes of RA that are responsive to agents such as rituximab, or other B cell antigens.

The most commonly used cytotoxic drugs for RA are azathioprine (Imuran), cyclophosphamide (Cytoxan) and cyclosporine A (Sandimmune). Because the potential of high toxicity, these agents are utilized for life-threatening extra-articular manifestations of RA such as systemic vasculitis or severe articular disease refractory to other therapy. It is recommended that these agents be used under the direction of a rheumatologist. Azathioprine is a purine analog. Cyclophosphamide is an alkylating agent. Cyclosporine is an immunosuppressive agent approved for use in preventing renal and liver allograft rejection. Cyclosporine inhibits T cell function by inhibiting transcription of interleukin-2. Main toxicity's include infection and renal insufficiency.

Interleukin-6 is a glycoprotein composed of 184 amino acids. Numerous cells can produce this inducible cytokine, including macrophages, B cells, T cells, fibroblasts, endothelial cells, mesangial cells, and many types of tumor cells. The effects of IL-6 are pleiotropic, occurring at both a systemic and a local tissue level, and involving a wide variety of cells. Of particular relevance to RA are the effects on the differentiation of B and T lymphocytes, as well as the differentiation of macrophages, megakaryocytes, and osteoclasts. Interleukin-6 is elevated in the serum and synovial fluid in RA patients. The excessive production of IL-6 is postulated to play a role in the pathogenesis of several inflammatory diseases such as RA, Crohn's disease, and juvenile idiopathic arthritis. In RA, IL-6 participates in immune cell activation and autoantibody production, osteoclastogenesis, and bone loss, and the often debilitating systemic and constitutional symptoms associated with the acute-phase response. MRA (Chugai Pharmaceutical Co. Ltd., Tokyo, Japan) is a humanized anti-IL-6 receptor antibody (Tocilizumab) that inhibits the binding of IL-6 to its receptor IL-6R and prevents IL-6 signal transduction.

Trials targeting other cytokines, including IL-12, IL-15, IL-18, and p19 subunit of IL-23 (Eli Lilly) are in clinical development. AMG 714 (Genmab, Copenhagen, Denmark) is a human monoclonal antibody that binds to IL-15 and inhibits its signaling. Patients receiving AMG 714 had clinically meaningful improvement compared with placebo, demonstrating that IL-15 is a target in the treatment of RA. In preclinical studies, an anti-IL-17 antibody significantly reduced the severity of collagen-induced arthritis. BlyS, or BAFF, is a member of the tumor necrosis factor family of cytokines, and its receptors, BCMA, BAFFR, and TACI, are largely restricted to B cells (a small amount of TACI has been found on activated T cells). LymphoStat-B is a fully human IgG1A monoclonal antibody that neutralizes human BlyS. The administration of LymphoStat-B to cynomolgus monkeys selectively reduces B cells in blood and tissue with no overt toxicity. Natalizumab (TYSABRI™, Biogen) is a monoclonal antibody specific for alpha-4-integrin and blocks the homing of white blood cells into tissues. Natalizumab was recently FDA approved for MS.

### Diagnostic and Prognostic Methods

The differential presence of specific autoantibodies is shown to provide for prognostic evaluations to detect individuals having clinical subtypes that correspond to responsiveness or non-responsiveness to treatments of interest, where the treatment of interest is other than an antigen-specific treatment, e.g. a DMARD. In general, such prognostic methods involve determining the presence or level of autoantibodies in an individual sample, usually a blood derived sample, e.g. blood, serum, plasma, etc. A variety of different assays can be utilized to quantitate the presence of autoantibodies. Many such methods are known to one of skill in the art, including ELISA, protein arrays, eTag system, bead based systems, tag or other array based systems etc. Examples of such methods are set forth in the art, including, inter alia, chip-based capillary electrophoresis: Colyer et al. (1997) J Chromatogr A. 781(1-2):271-6; mass spectroscopy: Petricoin et al. (2002) Lancet 359: 572-77; eTag systems: Chan-Hui et al. (2004) Clinical Immunology 111:162-174; microparticle-enhanced nephelometric immunoassay: Montagne et al. (1992) Eur J Clin Chem Clin Biochem. 30(4):217-22; antigen arrays: Robinson et al. (2002) Nature Medicine, 8:295-301; the Luminex XMAP bead array system (www.luminexcorp.com); and the like, each of which are herein incorporated by reference. Detection may utilize one or a panel of specific binding members, e.g. specific for at least about 5, at least about 10, at least about 15, at least about 20 or more distinct autoantigen peptides.

The signature pattern may be generated from a biological sample using any convenient protocol, for example as described below. The readout may be a mean, average, median or the variance or other statistically or mathematically-derived value associated with the measurement. The antigen or epitope readout information may be further refined by direct comparison with the corresponding reference or control pattern. A binding pattern may be evaluated on a number of points: to determine if there is a statistically significant change at any point in the data matrix; whether the change is an increase or decrease in the epitope binding; whether the change is specific for one or more physiological states, and the like. The absolute values obtained for each epitope under identical conditions will display a variability that is inherent in live biological systems and also reflects individual antibody variability as well as the variability inherent between individuals.

Following obtainment of the signature pattern from the sample being assayed, the signature pattern is compared with a reference or control profile to make a prognosis regarding the phenotype of the patient from which the sample was obtained/derived. Typically a comparison is made with a sample or set of samples from an unaffected, normal source. Additionally, a reference or control signature pattern may be a signature pattern that is obtained from a sample of a patient known to be responsive or non-responsive to the therapy of interest, and therefore may be a positive reference or control profile.

In certain embodiments, the obtained signature pattern is compared to a single reference/control profile to obtain information regarding the phenotype of the patient being assayed. In yet other embodiments, the obtained signature pattern is compared to two or more different reference/control profiles to obtain more in depth information regarding the phenotype of the patient. For example, the obtained signature pattern may be compared to a positive and negative reference profile to obtain confirmed information regarding whether the patient has the phenotype of interest.

Samples can be obtained from the tissues or fluids of an individual. For example, samples can be obtained from whole blood, tissue biopsy, serum, etc. Other sources of samples are body fluids such as synovial fluid, lymph, cerebrospinal fluid, bronchial aspirates, and may further include saliva, milk, urine, and the like. Also included in the term are derivatives and fractions of such cells and fluids. Diagnostic samples are collected any time after an individual is suspected to have an autoimmune disease or has exhibited symptoms that predict such a disease. Optionally the sample is treated to block or deplete heterophilic antibodies, e.g. RF, as exemplified in the examples.

Various immunoassays designed to quantitate antibodies may be used in screening. Measuring the concentration of the target protein in a sample or fraction thereof may be accomplished by a variety of specific assays. For example, a conventional sandwich type assay may be used in an array, ELISA, RIA, etc. format. A sandwich assay may first attach specific autoantigen peptides to an insoluble surface or support. The particular manner of binding is not crucial so long as it is compatible with the reagents and overall methods of the invention. They may be bound to the plates covalently or non-covalently.

The insoluble supports may be any compositions to which polypeptides and other biomolecules can be bound, which is readily separated from soluble material, and which is otherwise compatible with the overall method. The surface of such supports may be solid or porous and of any convenient shape. Examples of suitable insoluble supports to which the receptor is bound include slides, beads, e.g. magnetic beads, membranes and microtiter plates. These are typically made of glass, plastic (e.g. polystyrene), polysaccharides, nylon or nitrocellulose.

Patient sample preparations are then added to the antigen containing substrate. Preferably, a series of standards, containing known concentrations of the test protein is assayed in parallel with the samples or aliquots thereof to serve as controls. Preferably, samples are assayed in multiple spots, wells, etc. so that mean values can be obtained for each. The incubation time should be sufficient for binding, generally, from about 0.1 to 3 hr is sufficient. After incubation, the insoluble support is generally washed of non-bound components. A dilute non-ionic detergent medium at an appropriate pH, generally 7-8, can be used as a wash medium. From one to six washes can be employed, with sufficient volume to thoroughly wash non-specifically bound proteins present in the sample.

After washing, a solution containing a detection reagent, e.g. antibodies reactive with human immunoglobulin, is applied. The second stage reagent may be labeled to facilitate direct, or indirect quantification of binding. Examples of labels that permit direct measurement of second receptor binding include radiolabels, such as ³H or ¹²⁵1, fluorescers, dyes, beads, chemiluminescers, colloidal particles, and the like. Examples of labels that permit indirect measurement of binding include enzymes where the substrate may provide for a colored or fluorescent product. In a preferred embodiment, the antibodies are labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The incubation time should be sufficient for the labeled ligand to bind available molecules. Generally, from about 0.1 to 3 hr is sufficient, usually 1 hr sufficing.

After the second binding step, the insoluble support is again washed free of non-specifically bound material, leaving the specific complex formed between the patient antibodies and the detection reagent. The signal produced by the bound conjugate is detected by conventional means. Where an enzyme conjugate is used, an appropriate enzyme substrate is provided so a detectable product is formed.

Other immunoassays are known in the art and may find use as diagnostics. Ouchterlony plates provide a simple determination of antibody binding. Western blots may be performed on protein gels or protein spots on filters, using a detection system specific for the autoimmune disease associated polypeptide as desired, conveniently using a labeling method as described for the sandwich assay.

In some cases, a competitive assay will be used. In addition to the patient sample, a competitor to the autoantigen is added to the reaction mix. The competitor and the autoantigen compete for binding. Usually, the competitor molecule will be labeled and detected as previously described, where the amount of competitor binding will be proportional to the amount of target antigen present. The concentration of competitor molecule will be from about 10 times the maximum anticipated protein concentration to about equal concentration in order to make the most sensitive and linear range of detection.

Alternatively, a reference sample may be used as a comparator. In such a case, the reference patient or antibody sample is labeled with or detected using a spectrally distinct fluorophore from that used to label or detect antibodies from the patient sample. This reference sample is mixed with the patient sample, and the mixed sample analyzed on antigen arrays or another antibody measurement methodology. Such an approach provides a ratio of patient:reference sample antibody binding to an individual antigen, thereby enabling direct comparative analysis of patient antibody binding relative to reference sample antibody binding to individual antigens.

For multiplex analysis of cytokines, arrays containing one or more anti-cytokine antibodies can be generated. Such an array is constructed comprising antibodies against cytokines, and may include antibodies binding cytokines listed in Table 2. Various immunoassays designed to quantitate cytokines may be used in screening. Measuring the concentration of the target protein in a sample or fraction thereof may be accomplished by a variety of specific assays. For example, a conventional sandwich type assay may be used in an array, ELISA, RIA, bead array, etc. format. A sandwich assay may first attach specific autoantigen peptides to an insoluble surface or support. The particular manner of binding is not crucial so long as it is compatible with the reagents and overall methods of the invention.

The detection reagents can be provided as part of a kit. Thus, the invention further provides kits for detecting the presence of a panel of autoantibodies in a biological sample. Procedures using these kits can be performed by clinical laboratories, experimental laboratories, medical practitioners, or private individuals. The kits of the invention for detecting antibodies comprise autoantigens useful for generating a prognostic signature pattern, which may be provided in solution or bound to a substrate. The kit may optionally provide additional components that are useful in the procedure, including, but not limited to, buffers, developing reagents, labels, reacting surfaces, means for detection, control samples, standards, instructions, and interpretive information.

### Diagnostic Arrays

Arrays provide a high throughput technique that can assay a large number of polypeptides in a sample. In one aspect of the invention, an array is constructed comprising one or more autoantigen peptides, which may include peptides provided in Table 1, preferably comprising peptides specific for at least 5 distinct epitopes, at least about 10, at least about 15, at least about 20, or more. This technology is used as a tool to quantitate antibody binding. Arrays can be created by spotting a peptide probe onto a substrate (e.g., glass, nitrocellulose, etc.) in a two-dimensional matrix or array having bound probes. The probes can be bound to the substrate by either covalent bonds or by non-specific interactions, such as hydrophobic interactions. Techniques for constructing arrays and methods of using these arrays are described in, for example, Schena et al. (1996) Proc Natl Acad Sci USA. 93(20):10614-9; Schena et al. (1995) Science 270(5235):467-70; Shalon et al. (1996) Genome Res. 6(7):639-45, U.S. Pat. No. 5,807,522, EP 799 897; WO 97/29212; WO 97/27317; EP 785 280; WO 97/02357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734.

The probes utilized in the arrays can be of varying types and can include, for example, peptide, peptidomimetics; lipid antigens, DNA antigens, and the like. Arrays can be utilized in detecting differential antibody binding levels. In one embodiment of the invention, the array comprises a plurality of autoantigens.

Common physical substrates for making arrays include glass or silicon slides, magnetic particles or other micro beads, functionalized with aldehyde or other chemical groups to help immobilize proteins. The substrate can also be coated with PLL, nitrocellulose, PVDF membranes or modified with specific chemical reagents to adsorb capture agents. The desirable properties of an ideal surface include: chemical stability before, during, and after the coupling procedure, suitability for a wide range of capture agents (e.g., hydrophilic and hydrophobic, low MW and high MW), minimal non-specific binding, low or no intrinsic background in detection, presentation of the capture agents in a fully-functional orientation, production of spots with predictable and regular morphology (shape, signal uniformity).

The variables in the immobilization of proteins include: type of capture agent, nature of surface (including any pretreatment prior to use), and the immobilization method. Both adsorption and covalent attachment have been used for protein arrays. Orientation of the capture agent is very important in presenting it to the ligand or the surface in a functional state. Although covalent attachment using a variety of chemically activated surfaces (e.g., aldehyde, amino, epoxy) as well as attachment by specific biomolecular interactions (e.g., biotin-streptavidin) provide a stable linkage and good reproducibility, chemical derivatization of the surface may alter the biological activity of the capture agent and/or may result in multi-site attachment.

In one embodiment, arrays of antigens and/or antibodies are made with a non-contact deposition printer. The printer uses thermal ink jet heads that can print many solutions simultaneously to produce hundreds of spots of 50-60 μm diameter with a spacing of 150 μm between spots. The droplet volume ranges between 35 pL to 1.5 nL. The heating element is made out of TaAl or other suitable materials, and is capable of achieving temperatures that can vaporize a sufficient volume of printing buffer to produce a bubble that will push out a precise volume of the antibody solution on the substrate. Selection of printing buffer is important, in that the buffer accomplishes the following: increases printing efficiency (measure of the number of spots that are printed to the total number of spots that are attempted), reduces sample spreading, promotes uniform delivery, stabilizes the capture agents that are being printed, reduces sample drying, increases the visibility of the printed spots. In addition to the printing buffer, other variables that affect printing include: size of the drops, the method of washing and drying the print head, and the speed at which the dispensing head moves. Various modifications may be within these conditions.

In another embodiment, arrays of antigens and/or antibodies are attached to fluorescently addressable beads or other addressable tags. Antigens or antibodies are incubated with the addressable beads or tags to conjugate them via covalent bonds, avidin-biotin binding, electrostatic forces or other binding mechanisms. Such an approach may be performed using the Beadlyte Human 22-Plex Cytokine Detection System (Upstate Biotechnology, Lake Placid, N.Y., USA) in conjunction with the Luminex 100 LabMAP System (Luminex, Austin, Tex., USA) for multiplex cytokine analysis.

Both direct labeling and sandwich format approaches may find use. In the direct labeling procedure, the antibody array is interrogated with serum samples that had been derivatized with a fluorescent label, e.g. Cy3, Cy5 dye, etc. In the sandwich assay procedure, unlabeled serum is first incubated with the array to allow target antibodies to be captured by immobilized capture antigens. Next, the captured target antibodies are detected by the application of a labeled detection reagent. The sandwich assay provides extra specificity and sensitivity needed to detect small concentrations of antibodies, without compromising the binding affinities of the antibodies through a direct labeling procedure.

Fluorescence intensity can be determined by, for example, a scanning confocal microscope in photon counting mode. Appropriate scanning devices are described by e.g., U.S. Pat. No. 5,578,832 to Trulson et al., and U.S. Pat. No. 5,631,734 to Stern et al. and are available from Affymetrix, Inc., under the GeneChip™ label. Some types of label provide a signal that can be amplified by enzymatic methods (see Broude, et al., Proc. Natl. Acad. Sci. U.S.A. 91, 3072-3076 (1994)). A variety of other labels are also suitable including, for example, radioisotopes, chromophores, magnetic particles and electron dense particles.

Those locations on the probe array that are bound to sample are detected using a reader, such as described by U.S. Pat. No. 5,143,854, WO 90/15070, and U.S. Pat. No. 5,578,832. For customized arrays, the hybridization pattern can then be analyzed to determine the presence and/or relative amounts or absolute amounts of known species in samples being analyzed as described in e.g., WO 97/10365.

Other methodologies also find use. Methods such as surface plasmon resonance (SPR) are being developed for label-free detection of antibody-antigen binding events, and can be applied in an array format to profile the specificity of autoantibody responses. SPR senses refractive index change of molecules bound to a metal surface, and thereby enables detection of autoantibody binding using resonance and without need for fluorescent tags, enzymatic reactions, secondary antibodies, or washing methods that are frequently used to detect reactive autoantibodies in an immunoassay. In some embodiments, a solution based methodology utilizes capillary electrophoresis (CE) and microfluidic CE platforms for detecting and quantitating protein-protein interactions, including antibody reactions with serum proteins associated with autoimmune disease. This technique can be performed easily by any laboratory with access to a standard CE DNA sequencing apparatus. With this methodology, a fluorescent marker (eTag reporter) is targeted to the analyte with one antibody, and a second sandwich antibody of different epitope specificity that is chemically coupled to a “molecular scissors” induces release of the fluorescent probe when both antibodies are in close apposition on the specific analyte. Quantitation then is focused on the liberated eTag, that is quantified with a standard DNA capillary sequencing device. The eTag Assay System can be used to measure the abundance of multiple proteins simultaneously. A critical feature of the assay is that the affinity agents (antibodies) are not immobilized on surfaces, as is required with array technologies. Solution-based binding eliminates surface-induced denaturation and non-specific binding, and improves sensitivity and reaction kinetics.

By combining different colors in the eTag reporters, both mobility and color may be used to dramatically increase the degree of multiplexing. Many binding reactions can be multiplexed in the same vessel, followed by CE to identify the released eTag reporters. Each released eTag reporter encodes the identity of the probe to which it was originally attached. As a result, it is straightforward to configure multiplexed assays to monitor various types of molecular recognition events, especially protein-protein binding.

### Kits and Devices

Also provided are reagents and kits thereof for practicing one or more of the above-described methods. The subject reagents and kits thereof may vary greatly. Reagents of interest include reagents specifically designed for use in production of the above described expression profiles of circulating protein markers associated with autoimmune conditions. Such devices or kits will include reagents that specifically identify one or more autoantibodies and/or cytokines as described above. Devices of interest include arrays as described above. Alternatively the reagents may be provided as a kit comprising reagents in a suspension or suspendable form, e.g. reagents bound to beads suitable for flow cytometry, and the like. Reagents of interest include reagents specific for autoantibody markers. Such reagents may include antigenic proteins or peptides, and the like; cytokine-specific antibodies or fragments thereof; and the like.

In alternative embodiments, the reagents are provided as a kit comprising reagents in a suspension or suspendable form, e.g. reagents bound to beads suitable for flow cytometry, and the like. For example, the reagents for detection may comprise a molecular “tag,” where the reagent is attached to a detectable label, or tag, which provides coded information about the reagent. In certain cases these tags can be cleaved from the element, and subsequently detected to identity the element. In another embodiment, a set of reagents are synthesized or attached to a set of coded beads, where each bead is linked to a distinct antigen or epitope, and where the beads are themselves coded in a manner that allows identification of the attached antigen or epitope. The use of a multiplexed microsphere set for analysis of clinical samples by flow cytometry is described in International Patent application no. 97/14028; and Fulton et al. (1997) Clinical Chemistry 43:1749-1756). It is also possible to use other addressable particles or tags (reviewed in Robinson et al. (2002) Arthritis Rheumatism 46:885-93).

In this type of “tag array,” where the antigen is bound to beads or microspheres, one may utilize flow cytometry for detection of binding. For example, microspheres having fluorescence coding have been described in the art, where the color and level of fluorescence uniquely identifies a particular microsphere. The antigen is thus covalently attached to a “color coded” object. A labeled antibody can be detected by flow cytometry, and the coding on the microsphere used to identify the bound antigen.

In yet another embodiment, surface plasmon resonance (SPR) imaging is utilized to detect autoantibody binding without the need for fluorescent, enzymatic, or other detection markers. SPR, which senses refractive index change of molecules bound to a metal surface, provides label-free detection for autoantibody binding, which eliminates additional reaction and washing steps associated with most conventional detection methods.

The kits may further include a software package for statistical analysis of one or more phenotypes, and may include a reference database for calculating the probability of classification. The kit may include reagents employed in the various methods, such as devices for withdrawing and handling blood samples, second stage antibodies, ELISA reagents; tubes, spin columns, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that may be present is a website address which may be used via the internet to access the information at a removed site. Any convenient means may be present in the kits.

### Assessment of Patient Outcomes

Patient outcomes and Responder status may be assessed using imaging-based criteria such as radiographic scores, clinical and laboratory criteria. Multiple different imaging, clinical and laboratory criteria and scoring systems have been and are being developed to assess disease activity and response to therapy in rheumatoid arthritis, systemic lupus erythmatosus, Crohn's disease, and many other autoimmune diseases.

In rheumatoid arthritis, response to therapy is conventionally measured using the American College of Rheumatology (ACR) Criteria. The ACR response criteria are a composite score comprising clinical (swollen joint count, tender joint count, physician and patient response assessment, and health assessment questionnaire), and laboratory (acute phase response) parameters, level of improvement is reported as an ACR20 (20%), ACR50 (50%) or ACR70 (70%) response, which indicates percent change (improvement) from the baseline score. A number of clinical trails based on which the anti-TNFα agents infliximab (Remicade™) etanercept (Enbrel™) and adalimumab (Humira™) were approved to treat human RA utilized ACR response rates as a primary outcome measure.

Responses in rheumatoid arthritis many also be assessed using other response criteria, such as the Disease Activity Score (DAS), which takes into account both the degree of improvement and the patient's current situation. The DAS has been shown to be comparable in validity to the ACR response criteria in clinical trials. The definitions of satisfactory and unsatisfactory response, in accordance with the original DAS and DAS28. The DAS28 is an index consisting of a 28 tender joint count, a 28 swollen joint count, ESR (or CRP), and an optional general health assessment on a visual analogue scale (range 0-100) (Clinical and Experimental Rheumatology, 23(Suppl. 39):593-99, 2005). DAS28 scores are being used for quantification of response mostly in European trials of (early) rheumatoid arthritis such as the COBRA or BeST studies.

Radiographic measures for response in RA include both conventional X-rays (plain films), and more recently magnetic resonance (MR) imaging, computed tomography (CT), ultrasound and other imaging modalities are being utilized to monitor RA patients for disease progression. Such techniques are used to evaluate patients for inflammatoin (synovitis), joint effusions, cartilage damage, bony erosions and other evidence of joint damage. Methotrexate, anti-TNF agents and DMARD combinations have been demonstrated to reduce development of bony erosions and other measures of joint inflammation and destruction in RA patients. In certain cases, such as with anti-TNF agents, healing of bony erosions has been observed.

For response to therapy in systemic lupus erythematosus there exist a variety of scoring systems including the Ropes system, the National Institutes of Health [NIH] system, the New York Hospital for Special Surgery system, the British Isles Lupus Assessment Group [BILAG] scale, the University of Toronto SLE Disease Activity Index [SLE-DAI], and the Systemic Lupus Activity Measure [SLAM] (Arthritis and Rheumatism, 32(9):1107-18, 1989). The BILAG assessment consists of 86 questions; some based on the patient's history, some on examination findings and others on laboratory results. The questions are grouped under eight headings: General (Gen), Mucocutaneous (Muc), Neurological (Cns), Musculoskeletal (Msk), Cardiovascular and Respiratory (Car), Vasculitis (Vas), Renal (Ren), and Haematological (Hae). Based on the answers, a clinical score is calculated. The SLEDAI is a weighted, cumulative index of lupus disease activity.

Crohn's disease activity may be measured using the Crohn's disease activity index (CDAI) (Gastroenterology 70:439-444, 1976). The CDAI is based on the 1. Number of liquid or very soft stools in one week, 2. Sum of seven daily abdominal pain ratings: (0=none, 1=mild, 2=moderate, 3=severe), 3. Sum of seven daily ratings of general well-being: (0=well, 1=slightly below par, 2=poor, 3=very poor, 4=terrible), 4. Symptoms or findings presumed related to Crohn's disease (arthritis or arthralgia, iritis or uveitis, erythema nodosum, pyoderma gangrenosum, apththous stomatitis, anal fissure, fistula or perirectal abscess, other bowel-related fistula, febrile (fever) episode over 100 degrees during past week), 5. Taking Lomotil or opiates for diarrhea, 6. Abnormal mass, and 7. Hematocrit [(Typical-Current)×6]. Other criteria and scoring systems may also be used.

### Diagnostic Algorithms

An algorithm that combines the results of multiple antibody specificity and/or cytokine level determinations and that will discriminate robustly between individuals that respond to a therapy of interest, which includes, without limitation, the response of rheumatoid arthritis patients to anti-TNFα treatment; and those that do not respond, and controls for confounding variables and evaluating potential interactions is used for prognostic and diagnostic purposes.

In such an algorithm, an autoimmune disease signature pattern is obtained as a dataset. The dataset comprises quantitative data for the presence in serum of at least 3 epitopes and/or cytokines, usually at least 5 epitopes and/or cytokines, more usually at least 10 epitopes and/or cytokines, and may include 15 or more epitopes and/or cytokines. The epitopes set forth in Table 1 and the cytokines set forth in Table 2 are exemplary for the analysis of rheumatoid arthritis. The dataset optionally quantitative data for the presence in a clinical sample of other markers, including the presence of cytokines, T cell presence or specificity, clinical indices, and the like.

In order to identify profiles that are indicative of responsiveness, a statistical test will provide a confidence level for a change in the expression, titers or concentration of markers between the test and control profiles to be considered significant, where the control profile may be for responsiveness or non-responsiveness. The raw data may be initially analyzed by measuring the values for each marker, usually in duplicate, triplicate, quadruplicate or in 5-10 replicate features per marker.

A test dataset is considered to be different than a control dataset if at least 3, usually at least 5, at least 10, at least 15 or more of the parameter values of the profile exceeds the limits that correspond to a predefined level of significance.

To provide significance ordering, the false discovery rate (FDR) may be determined. First, a set of null distributions of dissimilarity values is generated. In one embodiment, the values of observed profiles are permuted to create a sequence of distributions of correlation coefficients obtained out of chance, thereby creating an appropriate set of null distributions of correlation coefficients (see Tusher et al. (2001) PNAS 98, 5116-21, herein incorporated by reference). This analysis algorithm is currently available as a software “plug-in” for Microsoft Excel know as Significance Analysis of Microarrays (SAM). The set of null distribution is obtained by: permuting the values of each profile for all available profiles; calculating the pairwise correlation coefficients for all profile; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value may be applied to the correlations between experimental profiles.

For SAM, Z-scores represent another measure of variance in a dataset, and are equal to a value of X minus the mean of X, divided by the standard deviation. A Z-Score tells how a single data point compares to the normal data distribution. A Z-score demonstrates not only whether a datapoint lies above or below average, but how unusual the measurement is The standard deviation is the average distance between each value in the dataset and the mean of the values in the dataset.

Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pairwise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation.

The data may be subjected to non-supervised hierarchical clustering to reveal relationships among profiles. For example, hierarchical clustering may be performed, where the Pearson correlation is employed as the clustering metric. One approach is to consider a patient autoimmune disease dataset as a “learning sample” in a problem of “supervised learning”. CART is a standard in applications to medicine (Singer (1999) Recursive Partitioning in the Health Sciences, Springer), which may be modified by transforming any qualitative features to quantitative features; sorting them by attained significance levels, evaluated by sample reuse methods for Hotelling's T² statistic; and suitable application of the lasso method. Problems in prediction are turned into problems in regression without losing sight of prediction, indeed by making suitable use of the Gini criterion for classification in evaluating the quality of regressions.

Other methods of analysis that may be used include logic regression. One method of logic regression Ruczinski (2003) Journal of Computational and Graphical Statistics 12:475-512. Logic regression resembles CART in that its classifier can be displayed as a binary tree. It is different in that each node has Boolean statements about features that are more general than the simple “and” statements produced by CART.

Another approach is that of nearest shrunken centroids (Tibshirani (2002) PNAS 99:6567-72). The technology is k-means-like, but has the advantage that by shrinking cluster centers, one automatically selects features (as in the lasso) so as to focus attention on small numbers of those that are informative. The approach is available as Prediction Analysis of Microarrays (PAM) software, a software “plug-in” for Microsoft Excel, and is widely used. Two further sets of algorithms are random forests (Breiman (2001) Machine Learning 45:5-32 and MART (Hastie (2001) The Elements of Statistical Learning, Springer). These two methods are already “committee methods.” Thus, they involve predictors that “vote” on outcome. Several of these methods are based on the “R” software, developed at Stanford University, which provides a statistical framework that is continuously being improved and updated in an ongoing basis.

Other statistical analysis approaches including principle components analysis, recursive partitioning, predictive algorithms, Bayesian networks, and neural networks.

These tools and methods can be applied to several classification problems. For example, algorithms can be developed from the following comparisons: i) all cases versus all controls, ii) all cases versus nonresponsive controls, iii) all cases versus responsive controls.

In a second analytical approach, variables chosen in the cross-sectional analysis are separately employed as predictors. Given the specific outcome, the random lengths of time each patient will be observed, and selection of proteomic and other features, a parametric approach to analyzing responsiveness may be better than the widely applied semi-parametric Cox model. A Weibull parametric fit of survival permits the hazard rate to be monotonically increasing, decreasing, or constant, and also has a proportional hazards representation (as does the Cox model) and an accelerated failure-time representation. All the standard tools available in obtaining approximate maximum likelihood estimators of regression coefficients and functions of them are available with this model.

In addition the Cox models may be used, especially since reductions of numbers of covariates to manageable size with the lasso will significantly simplify the analysis, allowing the possibility of an entirely nonparametric approach to survival.

These statistical tools are applicable to all manner of antibody specificity data. A set of data that can be easily determined, and that is highly informative regarding detection of individuals with clinically significant responsiveness to therapy is provided.

Also provided are databases of signature patterns for responsiveness of autoimmune patients to non-antigen specific therapies. Such databases will typically comprise signature patterns of individuals having responsive phenotypes, non-responsive phenotypes, etc., where such profiles are as described above.

The analysis and database storage may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data may be used for a variety of purposes, such as patient monitoring, initial diagnosis, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language may be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

The signature patterns and databases thereof may be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. All technical and scientific terms used herein have the same meaning as commonly understood to one of ordinary skill in the art to which this invention belongs unless clearly indicated otherwise.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

All publications and patent applications cited in this specification are herein incorporated by reference as if each individual publication or patent application were specifically and individually indicated to be incorporated by reference.

### EXPERIMENTAL

### Example 1

### Antigen Microarray Profiling of Autoantibodies and Bead Array Profiling of Cytokines Identifies “High-Inflammatory” Severe Arthritis and “Low-Inflammatory” Mild Arthritis Subtypes of Rheumatoid Arthritis

Rheumatoid arthritis (RA) is a polysynovitis of presumed autoimmune etiology that affects 0.6 to 1% of the population. Despite decades of research, the autoantigen targets and the molecular basis of RA remain poorly understood. The observed heterogeneity of disease manifestations, clinical course, and treatment responses suggests that unappreciated subtypes of RA exist on the molecular level. For example, a subpopulation of RA patients develops autoantibodies against citrullinated epitopes such as those represented by cyclic citrullinated peptide (CCP), which is associated with erosive disease. Another example is the heterogeneity in responsiveness to tumor necrosis factor alpha antagonist therapy. The advent of proteomics technologies has enabled large-scale analysis of proteins to identify biomarkers that delineate disease subtypes of RA, and to gain insights into the mechanisms underlying these subtypes.

For T cell-mediated autoimmune diseases, the presence of serum autoantibodies can predate the onset and be predictive of the development of clinical symptoms. In asymptomatic patients and in patients with undifferentiated arthritis, the presence of anti-CCP antibodies is a predictor of progression to RA. Detection of anti-CCP antibodies has been shown to provide a sensitivity of approximately 50-85% and a specificity of approximately 95% for the diagnosis of established RA. The process of citrullination is the result of the posttranslational conversion of arginine to citrulline by a family of enzymes termed peptidyl arginine deiminases (PADs). Vimentin and fibrinogen are considered candidate autoantigens in RA, based on the presence of these proteins in rheumatoid joints and the presence of autoantibodies against the citrullinated forms of these proteins in subpopulations of RA patients.

Autoantibodies targeting native proteins have also been described in RA. These include reactivities against heat-shock proteins (including Hsp65, Hsp90, DnaJ, and BiP), heterogeneous nuclear RNPs (hnRNP) A2/B1 (RA33) and D, annexin V, calpastatin, type II collagen, glucose-6-phosphate isomerase (GPI), elongation factor, and human cartilage gp39. Nevertheless, our understanding of the specificities of the autoimmune responses in RA remains limited.

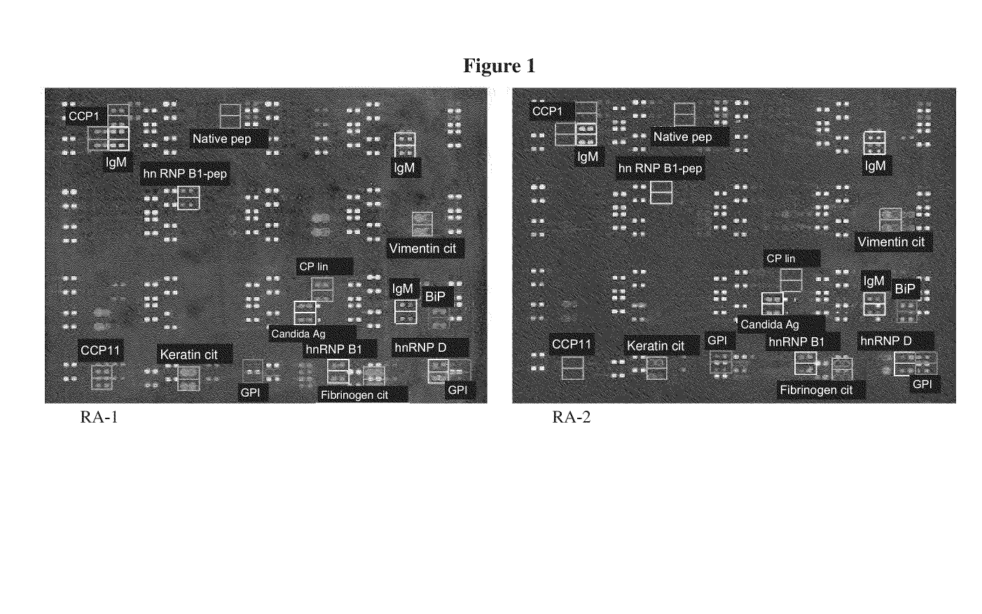

We developed and applied antigen microarrays for the diagnosis and classification of RA and early RA (FIG. 1) (Hueber et al. Arthritis Rheum 2005; 52:2645-55; Hueber et al. Clin Exp Rheumatol 2003; 21:S59-64, each specifically incorporated by reference). Hueber et al (Arthritis Rheum 2005; 52:2645-55) describes 1536-feature arthritis antigen arrays that contain 225 peptides and proteins representing candidate autoantigens in RA (FIG. 1). Antigens represented on arthritis arrays included native and in vitro citrullinated keratin, filaggrin, vimentin, and fibrinogen, a spectrum of heat shock proteins (HSP 65, 70, 90 and BiP); glucose 6 phosphate isomerase (GPI); collagen type I, II, III, IV and V; heterogeneous nuclear ribonucleoprotein (hnRNP) A2/RA33; and human cartilage gp39. Arrays also included peptides representing native human cartilage gp39, hnRNP-B1, and native and citrullinated epitopes derived from filaggrin, vimentin and fibrinogen. These antigens were robotically attached in ordered arrays to the surface of microscope slides where the binding of serum autoantibodies was detected.

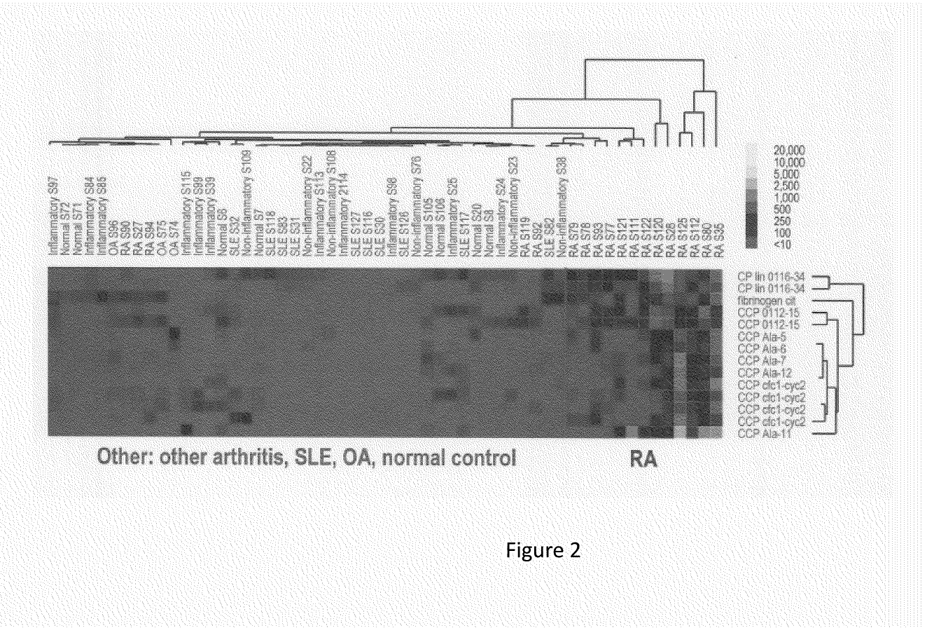

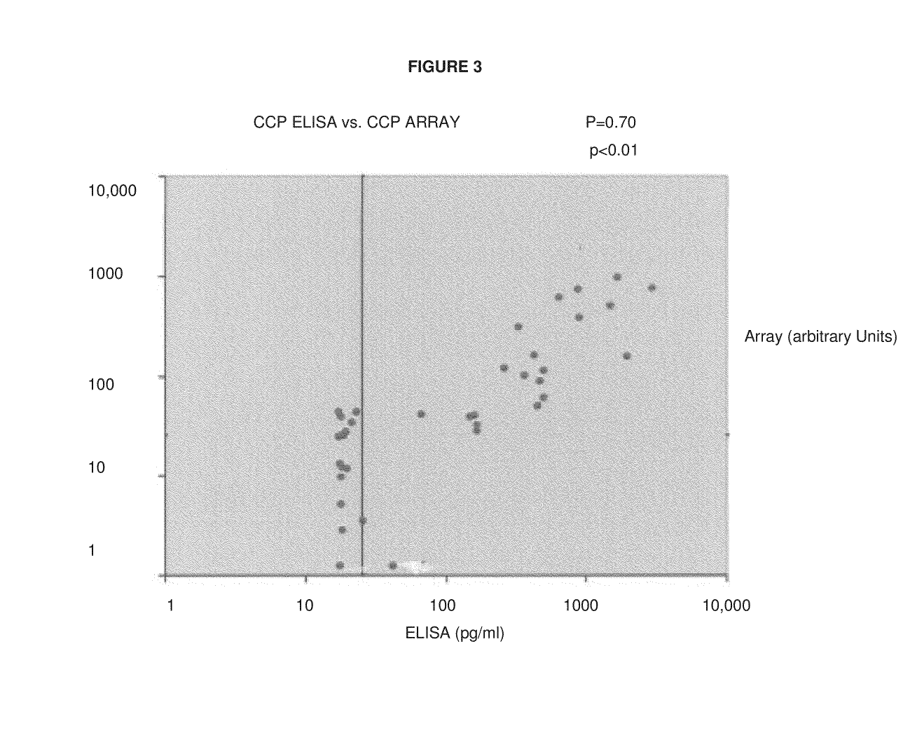

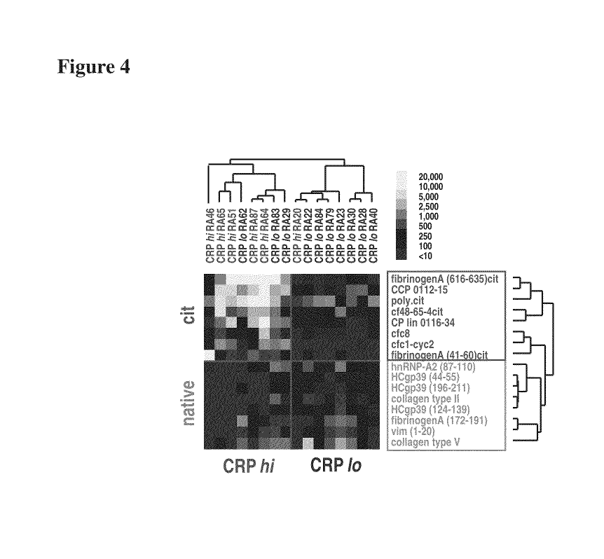

We observed preeminent reactivity against a spectrum of citrulline-containing epitopes in the ARAMIS inception cohort of early RA (samples analyzed were obtained within 6 months of the clinical diagnosis of RA) (FIG. 2), consistent with several recent reports that examined targeting of citrullinated antigens in RA. Array-detected reactivity against one particular cyclic citrullinated peptide (CCP) correlated well with results obtained using a commercial CCP2 ELISA assay (Axis-Shield Corporation) (FIG. 3). Moreover, anti-citrulline reactivity was associated with the C-reactive protein (CRP) inflammatory marker (FIG. 4). In contrast, we observed lower-level array reactivity against multiple unmodified, non-citrullinated autoantigens, including heat shock proteins, hnRNP A2/RA33, GPI and human cartilage gp39 in patients with low CRP levels (FIG. 4).

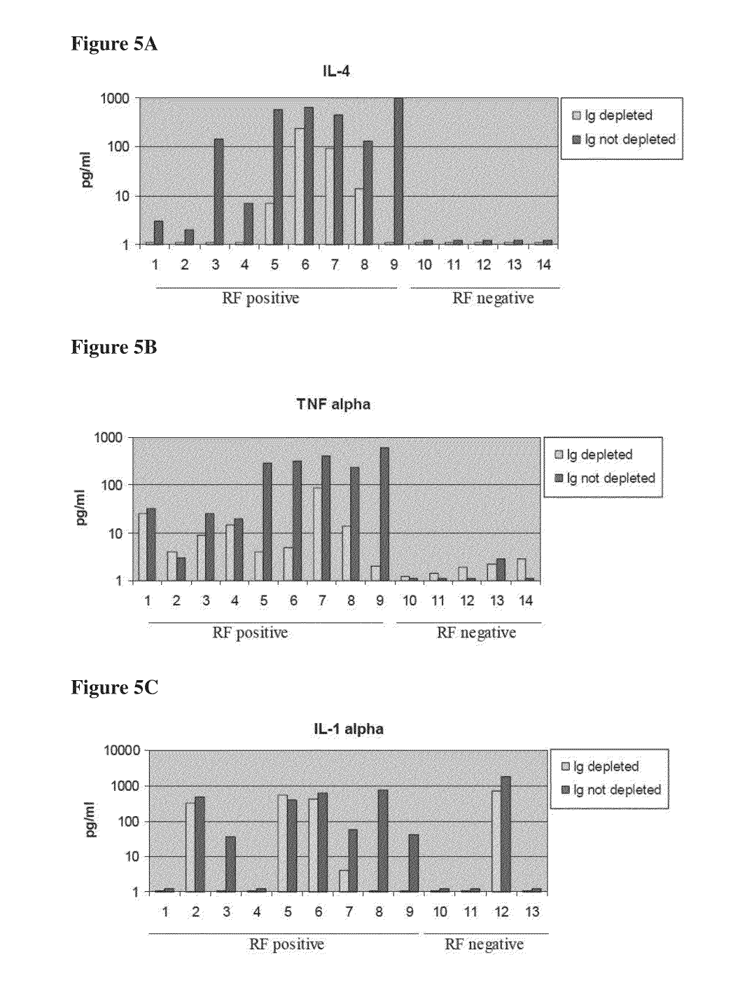

We further describe multiplex analysis of serum cytokine levels using a cytokine bead assay (FIGS. 5-7) and integration of these datasets with above-determined antigen array-derived autoantibody signatures, to investigate associations of distinct autoantibody profiles with cytokine profiles in patients from the ARAMIS early arthritis inception cohort. We tested the hypotheses that (i) cytokines derived from subsets of immunoregulatory cells are selectively upregulated in a subset of patients with early RA, and (ii) classes of cytokines are associated with distinct patterns of autoantibody reactivity.

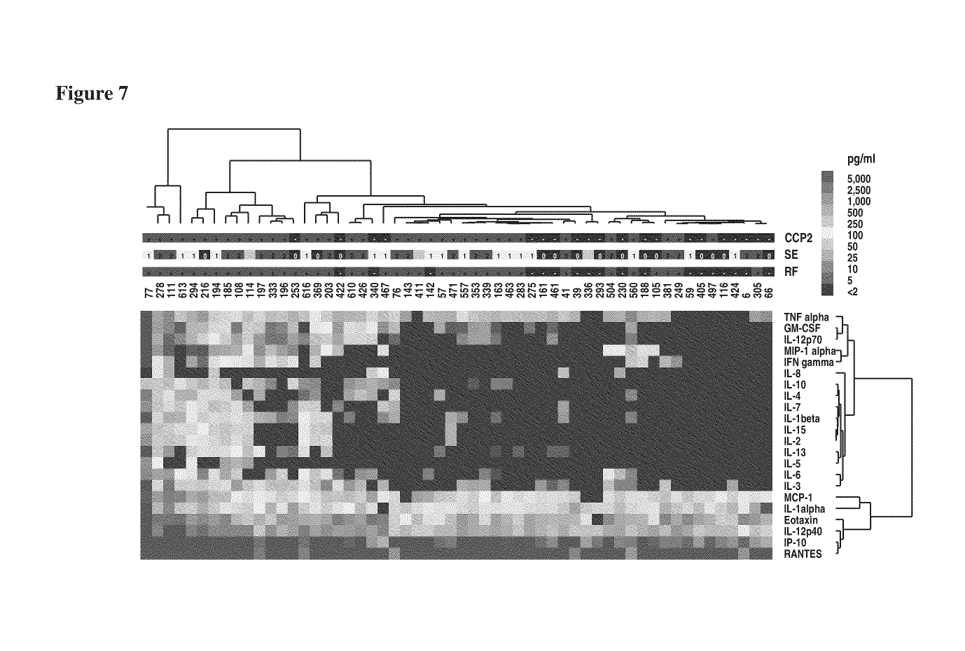

Analysis of the proteomic datasets of blood proteins revealed associations of distinct antibody profiles, including prominent anti-citrulline epitope reactivity, with the subgroup of early RA patients who exhibited high serum levels of the proinflammatory cytokines TNFα, IL-1, IL-6, IL-15 and GM-CSF, and IL-13. These proteomic profiles also correlated with surrogate markers predictive for the development of more severe arthritis including elevated CRP levels (FIG. 4) as well as the presence of rheumatoid factor (RF) and the shared epitope MHC polymorphism (FIG. 7). Several cytokines were broadly unregulated in the serum of a subgroup of early arthritis patients as compared to healthy individuals and control patients, but no Th1 or Th2 distribution was discernable (FIG. 7). Our results demonstrate associations of anti-citrulline autoantibody responses with production of proinflammatory cytokines, and these proteomic profiles are present in patients with clinical and laboratory features predictive for development of more severe arthritis.

In summary, our results demonstrate the presence of a “high-inflammatory” severe arthritis subtype of early RA characterized by anti-citrulline autoantibody responses and elevated cytokine levels, as well as a “low-inflammatory” mild arthritis subtype of early RA characterized by autoantibody responses targeting native antigens and low or normal blood cytokine levels.

### Patients and Methods

Patients and Sera.

All RA and control serum samples were obtained under Stanford IRB approved protocols and with informed consent. Serum samples from patients with ankylosing spondylitis and psoriatic arthritis (n=21), and from healthy individuals (n=19), were provided by a clinical reference laboratory (RDL Inc., Los Angeles, Calif.). Due to limitations in the number of arrays run in individual experiments, the Arthritis, Rheumatism, and Aging Medical Information System (ARAMIS) cohort samples studied comprised 56 randomly selected serum samples from 793 patients in the ARAMIS early RA inception cohort. These samples were collected from patients with a clinical diagnosis of RA (according to the revised American College of Rheumatology 1987 criteria) for <6 month's duration. The baseline characteristics of this subgroup of ARAMIS early RA patients were comparable to those of the whole cohort of patients, and their serum autoantibody responses were characterized in detail by antigen microarray assays.

TABLE 3
Baseline characteristics of the ARAMIS patients analyzed on arthritis
arrays and with a multiplex cytokine assay (n = 56)*

| | | |
|---|---|---|
| Age, median (range) years | 53.5 | (19-78) |
| Female sex, no. (%) | 43 | (77) |
| RF seropositive, no. (%) | 38 | (68) |
| CRP median (range) mg/dl | 0.50 | (0.09-15.7) |
| Median (range) disability score | 1.125 | (0-2.375) |
| Median (range) educational level score | 12 | (8-17) |
| DMARD treatment, no. (%) | 23 | (41) |
| Shared epitope present, no (%) | 38 | (68) |

*ARAMIS = Arthritis, Rheumatism and Aging Medical Information System; RF = rheumatoid factor; CRP = C-reactive protein; DMARD = disease-modifying antirheumatic drug.

Bead-Based Cytokine Assay.

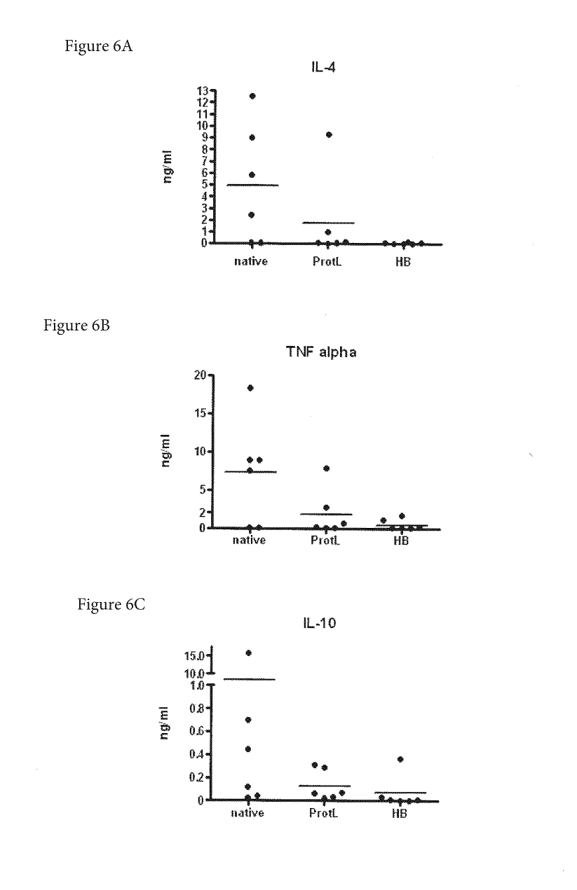

The human 22-cytokine Beadlyte™ kit (Upstate, Charlottesville, Va.) and the Luminex xMAP 100IS platform (Luminex, Austin, Tex.) were used for multiplex cytokine analysis (Assays were performed according to the manufacturer's protocol, except for using 50% of the recommended volumes (i.e., 12.5 μl of serum instead of 25 μl, and using an additional blocking reagent optimized for sandwich immunoassays (HETEROBLOCK™, Omega Biologicals Inc, Bozeman, Mont.), which was added to serum sample buffer to achieve 5 μg/ml final concentration) (applied in FIGS. 6-8 and 12-15). Immunodepletion of sera was performed by incubation of 100 μl of serum with 25 μl of protein L-sepharose beads (Pierce Biotechnologies, Rockford, Ill.) for 30 min at 4° C., followed by 30 sec centrifugation at 14 k RPM and removal of the supernatant for cytokine analysis (applied in FIGS. 5 and 6). Calibration controls and recombinant standards were used to generate standard curves as specified by the manufacturer. Linear (Pearson) correlation coefficients were calculated using InStat™ software (GraphPad Software Inc., San Diego, Calif.).

Construction, Probing and Scanning of Microarrays.

Detailed protocols for array production and data analysis were presented in prior work (Robinson et al, (2002) Nature Medicine, 8:295-301) and are available at www.stanford.edu/group/robinsonlab. Previously generated antigen array datasets were integrated with newly generated cytokine array datasets for the analysis of associations between autoantibody profiles and cytokine profiles in early RA.

Briefly, 0.2 mg/ml dilutions of antigens were printed in ordered arrays on poly-L lysine coated glass slides (CEL Associates, Pearland, Tex.) or ARRAYIT™ SuperExpoxy slides (TeleChem International, Sunnyvale, Calif.) as previously described. Arrays were circumscribed with a hydrophobic marker pen, blocked overnight in 3% fetal calf serum in PBS containing 0.05% Tween-20, probed with 300 μl of 1:150 dilutions of RA serum, washed, and incubated with a 1:4000 dilution of Cy3-conjugated goat anti-human IgG/M secondary antibody (Jackson lmmunoresearch, West Grove, Pa.). Arrays were scanned using the GenePix4000 Scanner. Median pixel intensities of features and background were determined using GenePix Pro 3.0 software (Molecular Devices, Union City, Calif.).

Arthritis Array Data Analysis.

Median net digital fluorescence units (DFUs) represent median values from 4-8 identical antigen features on each arthritis antigen array were normalized to the median intensity of 12-20 anti-IgM features. SAM identified antigens with statistical differences in array reactivity between samples derived from subgroups of RA patients. SAM ranks each antigen based on the difference in mean array reactivity between the groups divided by a function of the standard deviation, and then permutes the repeated measurements between groups to estimate a false discovery rate (FDR) for each antigen. Normalized median array values were mathematically adjusted and input into SAM, and SAM results were arranged into relationships using Cluster™ software, and Cluster results displayed using TreeView™ software.

ELISA.

The CCP2 ELISA kits (Immunoscan RA Mark 2, Eurodiagnostica, Malmo, Sweden) were used in accordance with the instructions of the manufacturer (FIG. 3).

Determination of the shared epitope status as well as other clinical and serologic parameters for the ARAMIS inception cohort are described in Fries et al. Arthritis Rheum 2002; 46:2320-9 (FIG. 7).

### Results

Serum Cytokine Profiles Stratify the RA Patient Population.

We performed analysis of serum cytokine levels using a multiplex cytokine assay. Samples with detectable cytokines frequently exhibited significant elevations of multiple cytokines, including both the classical Th1 (IFNγ and IL-12) and Th2 (IL-10 and IL-13) cytokines. Linear (Pearson's) correlation analysis of log-transformed cytokine concentrations showed strong correlations among Th1 cytokines (for example, IFNγ and IL-12p40, R=0.91) and Th2 cytokines (for example, IL-4 and IL-10, R=0.79). Moderate to strong correlations were also observed between Th1 and Th2 cytokines, for example IFNγ and IL-10, (R=0.65); IFNγ and IL-6, (R=0.65); and IL-12p70 and IL-10, (R=0.84). Correlations between TNFα and other pro-inflammatory cytokines were the strongest, with R values frequently >0.95 (TNFα and IL-15, (R=0.99); TNFα and GM-CSF, (R=0.99)).

Serum levels of multiple cytokines were associated with expression of anti-CCP2 antibodies and RF. Linear correlation analysis was performed to determine the magnitude of association between anti-CCP2 antibody titers and individual cytokine concentrations. The strongest correlations were found between anti-CCP2 titers and TNFα (R=0.39; p<0.007), IL-10 (R=0.38, p=0.004), IL-1β (R=0.37; p<0.005), IL-1α (R=0.36; p<0.007), and IL-4 (R=0.35, p=0.008), while weaker correlations were also observed with the chemokine IP-10/CXCL10 (R=0.32, p=0.02) and GM-CSF (R=0.31; p<0.02). Of note, IL-6, IL-13, IL-15, IL-8, MIP-1a/CCL3, MCP-1/CCL2 and eotaxin/CCL11, did not correlate with anti-CCP2 titers. RF titers demonstrated similar or stronger correlations with cytokine concentrations across the panel of cytokines tested, with strongest correlations for the pro-inflammatory cytokine IL-1β (R=0.54, 95% CI 0.33-0.71, p<0.0001). Anti-CCP2 ELISA positivity correlated moderately to strongly with positive RF results (Spearman R=0.76, 95% CI 0.62-0.86; p<0.0001). In summary, anti-CCP2 ELISA and RF titers were weakly correlated with concentrations of multiple cytokines, and no preferential association with Th1 or proinflammatory cytokines was observed. Correlations were also observed between CRP levels and IL-6 concentrations (Spearman R=0.42; 95% CI 0.17-0.63; p=0.001), and between CRP and MIP-1a/CCL3 concentrations (Spearman R=0.38; 95% CI 0.13-0.59; p=0.003), but not with the other cytokines measured.

Blocking of Heterophilic Antibodies is Required for Quantitative Measurements of Cytokines in RF Seropositive Sera.

The bead-based multiplex cytokine assay was validated by others, using both human serum and human peripheral blood mononuclear cell culture supernatants. Several groups reported that multiplexed assays were more reproducible and reliable than conventional ELISA-based measurements. However, concerns about the accuracy of measurements due to interfering factors, such as heterophilic antibodies and soluble cytokine receptors, in the serum matrix are a matter of ongoing debate for both multiplex assays and conventional ELISA. Heterophilic antibodies are defined as antibodies with multispecific activities directed against poorly defined antigens, and RF is classified as a heterophilic antibody. Multiple studies have demonstrated that blocking of heterophilic antibodies resulted in major reductions in the signal present in cytokine ELISAs, suggesting that heterophilic antibodies including RF can result in false-positive signals in ELISA and other immunoassays.

In preliminary experiments, we observed a striking association of elevated serum concentrations of multiple cytokines with RF seropositivity. To determine if RF was resulting in falsely elevated signal in the multiplex cytokine assay, we depleted serum of immunoglobulins by incubation of 100 μl of serum with 25 μl of protein L-sepharose beads for 30 min at 4° C. Depletion of RF and other potential heterophilic antibodies resulted in reductions of signal detected in ELISA for multiple but not all cytokines by up to 100-fold in several RF seropositive samples, as exemplified for IL-4, TNFα and IL-1α (FIG. 5). Measurements in RF seronegative samples were not affected.

Since depletion of immunoglobulin is a tedious and difficult to standardize procedure, we then tested a specially formulated blocking solution (HeteroBlock™). Heteroblock is a reagent optimized to prevent RF from bridging capture and detection antibodies in sandwich immunoassays. This blocking agent was reported previously to reduce non-specific binding of RF to primary and secondary antibodies in ELISA. In our experiments, the blocking effect was dependent on the concentration of HeteroBlock™ in the sample buffer, and a 1:175 dilution of HeteroBlock™ in the sample buffer was comparable to the effect of immunoglobulin depletion by protein L-sepharose precipitation (FIG. 6). This result indicates that the recommended proprietary blocking solution provided with the multiplex kit was insufficient to prevent false elevations in cytokine measurements in RF-seropositive samples, suggesting that more aggressive blocking, or alternatively, immunoglobulin removal by protein L-sepharose precipitation, is necessary for accurate quantification of cytokines in RF seropositive serum samples analyzed using this multiplex cytokine assay.

Serum Concentrations of the Cytokines IL-1α, TNFα, IL-12p40 and IL-13 are Elevated in Patients with Early RA as Compared to Controls.

Comparisons of cytokine concentrations between early RA, PsA/AS and healthy controls were performed using Kruskal-Wallis tests, with post-test analysis by Dunn's multiple comparisons. Serum concentrations of the following cytokines were significantly elevated in serum from patients with early RA: IL-1α (p<0.0001), TNFα (p<0.0001), IL-12p40 (p<0.0001), and IL-13. Significant differences were not observed for IL-6, for which median concentrations did not differ in PsA/AS and early RA. Concentrations of none of the remaining cytokines were significantly lower in the early RA group as compared to the healthy control and PsA/AS groups.

Serum Concentrations of the Chemokines IP-10/CXCL10, MCP-1/CCL2 and Eotaxin/CCL11 are Elevated in Patients with Early RA as Compared to Controls.

Median serum concentrations of three chemokines were higher in early RA:Eotaxin/CCL11 (p<0.0001), MCP-1/CCL2 (p=0.001) and IP-10/CXCL10 (p<0.001). IL-8 was the only cytokine with higher median concentrations in PsA/AS patients as compared to early RA patients (p=0.02).

Targeting of Citrullinated Autoantigens in Patients with Elevated Proinflammatory Cytokines Levels

Integration of Autoantibody Profiles with Cytokine Concentrations:

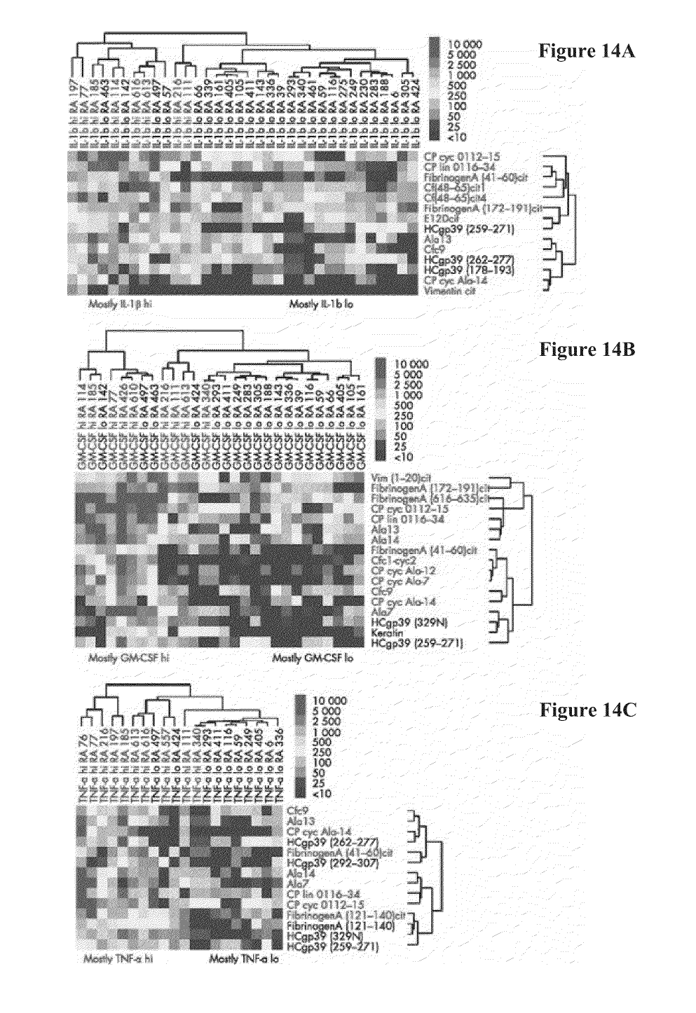

To integrate cytokine profiles with autoantibody profiles, we performed pairwise SAM analysis of arthritis array results from patients stratified based on the presence of elevated (cut-off 75^{th} percentile) and low/immeasurable serum concentrations of cytokines. In this cross sectional dataset, we determined that the proinflammatory cytokines IL-1β, TNFα, IL-6, IL-15, GM-CSF, and the Th2 cytokine IL-13 were associated with distinct antibody profiles (FIGS. 7 and 14) and surrogate markers of disease activity and severity (CRP and HAQ) in early RA. Patients were stratified according to sex and RF. These analyses revealed significantly increased autoantibody reactivity predominantly against citrullinated epitopes in patients within the cytokine^{high} subgroup, and subset analysis of women alone demonstrated even stronger correlations. Overall, the cytokine^{low} subgroup of patients demonstrated minimal reactivity against citrullinated epitopes, and no distinct antibody profile was identified in the cytokine^{low} subgroup (FIGS. 7 and 14).

Within the cytokine^{high} subgroup, CRP levels and self-reported HAQ scores were variably associated with higher concentrations of proinflammatory cytokines. Self reported HAQ scores were higher in the IL-18^{high} subgroup of women (p=0.01). Elevated CRP levels were seen in the IL-6^{high} subgroup (p=0.05) and TNFα^{high} subgroup (p=0.017), but neither HAQ scores nor CRP values varied significantly between the GM-CSF^{high} and GM-CSF^{low} patient subgroups (p >0.1). Thus, proinflammatory cytokines were consistently associated with distinct antibody profiles including prominent anti-citrulline reactivity, and variably associated with higher HAQ scores and elevated CRP values in early RA. Moreover, these observations were made with even lower FDR in the RF seropositive subgroup, but not in the seronegative subgroup of women.

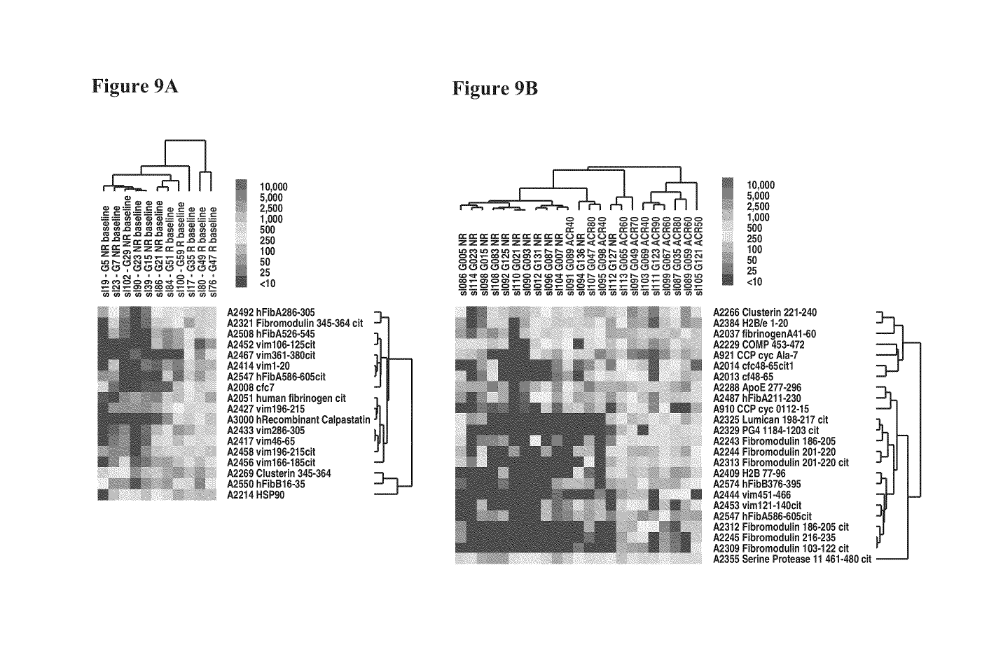

We describe herein the application of arthritis antigen microarrays and a bead-based cytokine assay to profile secreted immunoregulatory proteins, including autoantibodies and cytokines, in sera derived from patients with early RA. We identified proteomic patterns of differential antigen recognition and cytokine production that were associated with clinical subtypes of RA. Several citrullinated epitopes and a few native human cartilage gp39 peptides were preferentially targeted by autoantibodies in patients with high serum levels of the pro-inflammatory cytokines TNFα, IL-1β, IL-6, IL-13, IL-15 and GM-CSF, and these patients possessed laboratory and clinical features predictive for the development of more severe arthritis. Except for TNFα, these findings were more pronounced in women as compared to the complete cohort including samples from both sexes. Moreover, our results suggest an important role for the amplifiers of inflammatory responses in early RA, since three major chemokines were upregulated in RA over controls (FIGS. 7 and 9), including IP-10/CXCL10, a ligand of CXCR3 associated with Th1 type reactions, eotaxin/CCL11, a ligand of CCR3 associated with Th2 type reactions, and MCP-1/CCL2. Proinflammatory cytokines were the dominant category of cytokines upregulated in early RA, and no distinct Th1 or Th2 distribution of the elevations in cytokine levels was observed.

Little is known about the association of serum autoantibodies with serum cytokines. In type 1 diabetes (T1D), a prototypical Th1-type autoimmune disease, the presence of serum autoantibodies against multiple islet antigens was associated with mediators of the innate immune system (increased IL-18, and decreased MCP-1 and MIF), rather than elevations of with the classical Th1 cytokines. In a study involving a small number of RA patients, a generalized upregulation of serum cytokine concentrations over healthy controls was observed. Another recent study in early undifferentiated arthritis showed correlations between elevated concentrations of multiple cytokines with clinical subtypes and anti-CCP ELISA antibody responses in early RA. These authors identified a multi-cytokine profile consisting of IL-4, TNFα, IL-12, IL-13 and MIP-113 to be associated with CCP status and more severe disease, indicating that the immune response was not polarized towards Th1 in this cohort.

We describe prominent associations of array detected anti-citrulline antibody reactivity with elevated serum concentrations of the proinflammatory cytokines TNFα, IL-1β, IL-6, IL-13, IL-15, and GM-CSF (FIGS. 7, 8 and 14). We did not observe statistically significant increases of the classical Th2 cytokine IL-4 in RA serum as compared to controls, which might be due to aggressive sample treatment with HeteroBlock™, which corrected for false elevations of several cytokines in RF seropositive samples. We observed elevated serum levels of both classical Th1 (IFNγ, and IL-12) and Th2 (IL-10, IL-13) cytokines in a subgroup of patients with RA.

Since patients in the ARAMIS cohort were not treated with anti-cytokine therapies at the time blood samples were obtained, serum concentrations in these samples may be more reflective of systemic levels of proinflammatory cytokines than in patients treated with cytokine antagonizing biological agents. Nevertheless, it is well established that the presence of cell surface and secreted receptors can interfere with cytokine detection and that anti-cytokine antibodies present in certain patients mask detection of blood cytokines. It is possible that serum cytokine levels reflect the level of immune cell activation in involved joints or lymphoid tissues. Our findings suggest that generation of autoantibodies against citrullinated epitopes and other antigens is linked predominantly to the production of high levels of proinflammatory cytokines by activated T cells, macrophages and other cells in RA.

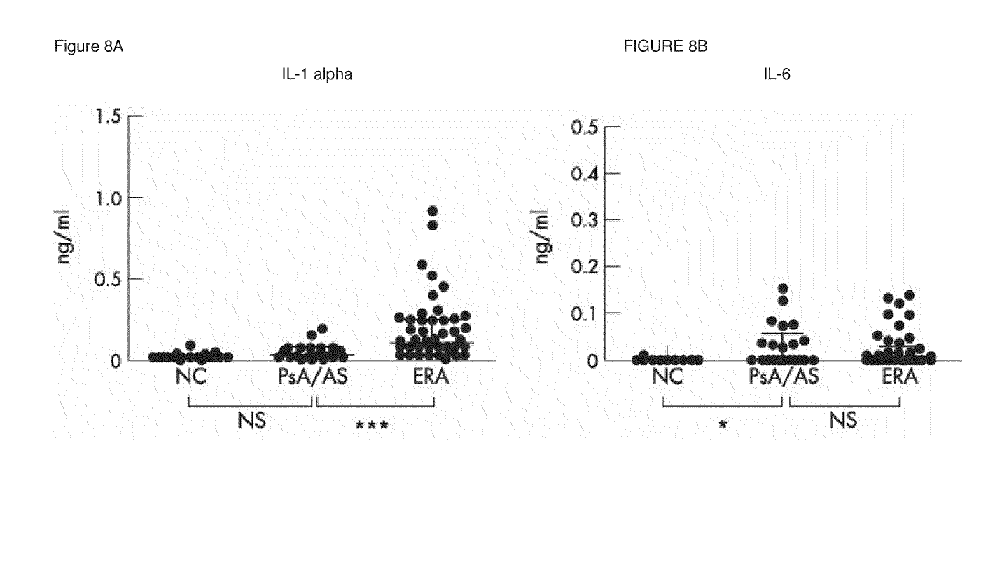

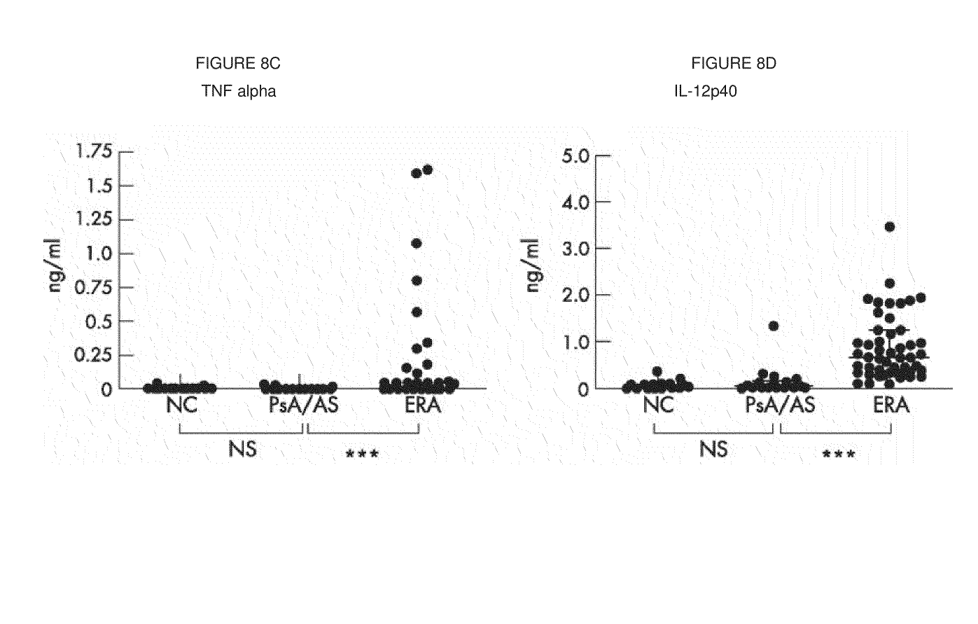

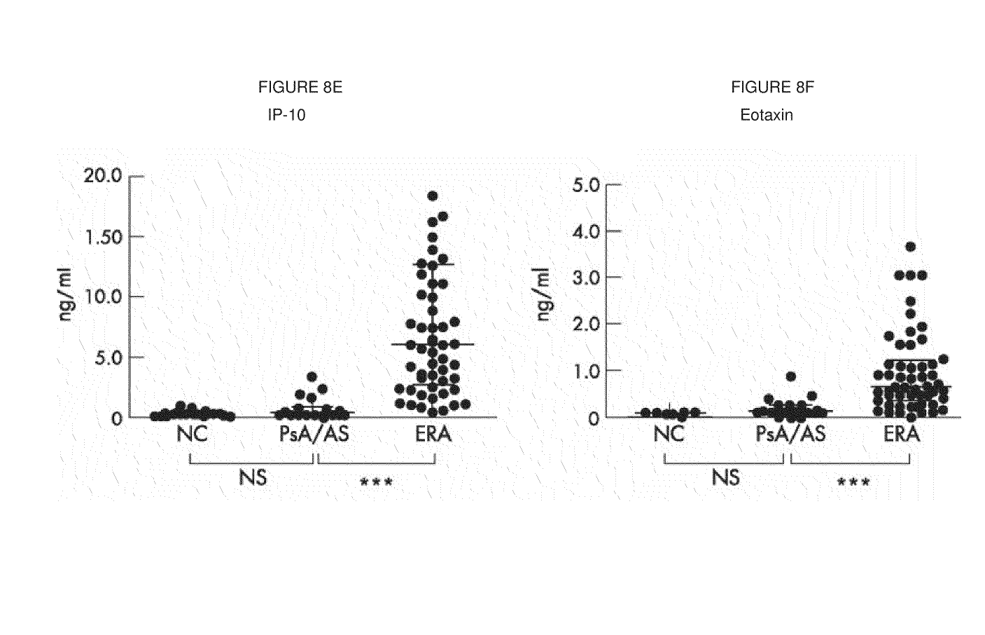

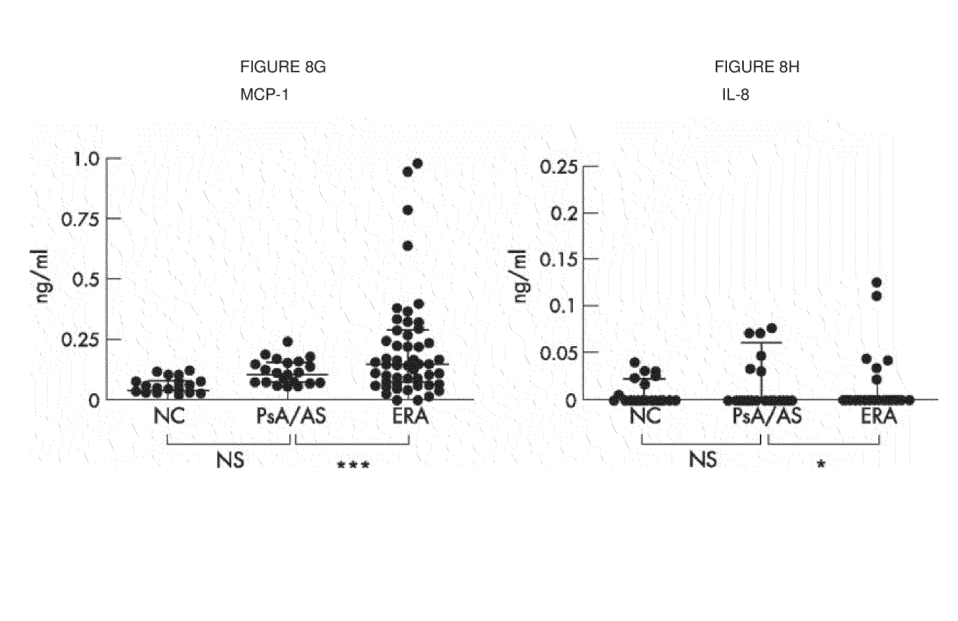

Recent work suggested that chemokines (a subset of the cytokine messenger molecules) play a prominent role in RA, and hence these modulators were proposed, together with their respective receptors, as targets for next generation therapeutics. Remarkably, our findings demonstrate that three chemokines, IP-10/CXCL10, eotaxin/CCL11 and MCP-1/CCL2, were strikingly elevated in early RA as compared to control patients (FIG. 8). The precise role of these molecules in chronic synovitis and cartilage destruction remains ill-defined. Chemokines such as IP-10/CXCL10 may have pivotal roles in recruiting activated T cells to sites of inflammation, and were proposed to be key mediators of T cell polarization in animal models of Th1-type autoimmunity. Other chemokines, including IL-8 and MCP-1, and CM-CSF are linked to TNFα via positive feedback loops and are additive or even synergistic in their biological and pathophysiological effects. We observed associations of GM-CSF levels with anti-citrulline reactivity. Since CM-CSF has been implicated in upregulation of class II MHC on human monocytes, an immunologic link between GM-CSF production, autoantigen presentation and induction of autoantibody production may exist.

Subsets of RF seronegative patients exhibited elevated levels of TNFα, IL-1α, IL-6 and IL-12p40 over healthy individuals, and these proinflammatory cytokines may be useful biomarkers in this subset of patients. Interestingly, concentrations of IL-12p70, the biologically active component of IL-12, did not appear to be different in RA versus controls, nor was it associated with distinct antibody profiles, in contrast to the more abundant IL-12p40. This suggests that one particular cytokine's utility as a biomarker is not exclusively dependent upon its biologically active molecular moiety.

Multiplex assays enable cost-effective and reliable simultaneous measurements of the levels of serum cytokines and autoantibodies in patients under evaluation with RA and other types of arthritis and autoimmunity. There is great need for the development of standardized multiplex assays to measure serum cytokines with ‘good laboratory practice’ (GLP) protocols. As robust assays become available for use in clinical laboratories, we anticipate that proteomic analysis will become a mainstay in the evaluation of RA and other autoimmune patients for assessing prognosis, guiding therapy and monitoring response to therapy.

### Patients and Methods

Antigens.

Many of the antigens were purchased from Sigma (St. Louis, Mo.), except for DnaJ and Hsp65, which were purchased from Stressgen, Victoria, British Columbia, Canada, and except for the following antigens, which were synthesized in our laboratories: recombinant hnRNP-B1 and hnRNP-D, recombinant BiP, mouse and human recombinant GPI, linear and cyclic citrulline-modified filaggrin peptides, overlapping peptides derived from human cartilage gp39, and overlapping peptides derived from hnRNP-A2. Additional native and citrulline-substituted 20-mer peptides derived from the fibrinogen a chain, vimentin, and filaggrin were synthesized (Sigma-Genosys, The Woodlands, Tex.).

In Vitro Citrullination.

Keratin, fibrinogen, and vimentin were citrullinated using rabbit muscle PAD (Sigma) as described previously. Successful citrullination was confirmed by Western blotting using rabbit anticitrulline antibodies (Upstate, Charlottesville, Va.). Antibodies. Monoclonal antibodies were purchased from Sigma (anti-Hsp65 and anti-Hsp70) or were generated in one of our laboratories (anti-La).

Sera.

All sera were collected under institutional review board-approved protocols and after provision of informed consent from the study subjects. The Stanford Arthritis Center samples were derived from 18 patients with established RA according to the American College of Rheumatology (ACR; formerly, the American Rheumatism Association) revised criteria for the classification of RA, 27 patients with arthritis in the setting of other autoimmune and nonautoimmune conditions, including systemic lupus erythematosus (SLE), ankylosing spondylitis, psoriatic arthritis, gout, and osteoarthritis, and 11 healthy controls. The Arthritis, Rheumatism, and Aging Medical Information System (ARAMIS) cohort comprised 58 randomly selected serum samples from the 793 patients in the ARAMIS early RA inception cohort. These samples were obtained from patients with a clinical diagnosis of RA of >6 months duration. Reference sera were provided for anti-CCP reactivity and for anti-hnRNP-B1, anti-hnRNP-D, and anti-Ro52/La reactivity.

Production of Antigen Microarrays.

Antigens were diluted to 0.2 mg/ml in phosphate buffered saline (PBS) or water and robotically attached in ordered arrays on derivatized poly-L-lysine-coated glass slides (CEL Associates, Pearland, Tex.) or Arraylt SuperEpoxy slides (TeleChem International, Sunnyvale, Calif.) as described previously. Individual antigen features had an average diameter of 200 μm.

Probing and Scanning of Autoantigen Arrays.

Arrays were circumscribed with a hydrophobic marker pap pen, blocked overnight with PBS containing 3% fetal calf serum and 0.05% Tween 20 (Sigma), and probed with 300 μl of 1:150 dilutions of RA or control patient serum, followed by washing and incubation with a 1:4,000 dilution of Cy3-conjugated goat anti-human IgG/IgM secondary antibody (Jackson ImmunoResearch, West Grove, Pa.). Arrays were scanned using the GenePix 4000 scanner, and the median pixel intensities of the features and background values were determined using Gene-Pix Pro version 3.0 software (Molecular Devices, Union City, Calif.).

Synovial Array Data Analysis.

Results of synovial arrays were expressed as normalized median net digital fluorescence units, representing the median values from 4-8 identical antigen features on each array normalized to the median intensity of 12-20 anti-IgM features. Significance analysis of microarrays (SAM) (Tusher et al. (2001) Proc. Natl. Acad. Sci. USA, 98:5116-21) was used to identify antigens with statistically significant differences in array reactivity between groups of patients with different diagnoses and between subgroups of patients with early RA. Using SAM, each antigen was ranked on the basis of differences in mean array reactivity between the groups, divided by a function of the standard deviation, and then repeated measurements between groups were permuted to estimate a false discovery rate (FDR) for each antigen. Normalized median array values were mathematically adjusted and inputed into SAM, with selection of results on the basis of various criteria for the respective experiments. SAM results were arranged into relationships using Cluster software, and the results from the Cluster analysis were displayed using TreeView software. For diagnostic class prediction, the Prediction Analysis of Microarrays (PAM) (2005, version 2.0; Proc Natl Acad Sci USA, 99:6567-72, 2004) algorithm was applied to synovial array results from the Stanford Arthritis Center cohort. PAM was used to identify a panel of antibody reactivities that characterized the diagnostic class of RA, and errors were estimated via crossvalidation. In a second analysis, we trained the PAM on array results from CCP-2-positive versus CCP-2-negative (by enzyme-linked immunosorbent assay [ELISA]) RA patients from one-half of the ARAMIS early RA sample set (n=29), and then used the second half of the samples (n=29) as a test set for subset class prediction.

ELISA.

An ELISA kit (Immunoscan RA Mark 2; Euro-Diagnostica, Malmoe, Sweden) was used to detect CCP-2, carried out in accordance with the specifications of the manufacturer.

Bead-Based Cytokine Assay.

The human 22-cytokine Beadlyte kit (Upstate, Charlottesville, Va.) and the Luminex xMAP 100IS platform (Luminex, Austin, Tex.) were used for multiplex cytokine analysis. Assays were performed according to the manufacturer's protocol, except for using 50% of the recommended volumes (i.e., 12.5 μl of serum instead of 25 μl, and using an additional blocking reagent optimized for sandwich immunoassays (HeteroBlock™, Omega Biologicals Inc, Bozeman, Mont.), which was added to serum sample buffer to achieve 5 mg/ml final concentration). Immunodepletion of sera was performed by incubation of 100 μl of serum with 25 μl of protein L-sepharose beads (Pierce Biotechnologies, Rockford, Ill.) for 30 min at 4° C., followed by 30 sec centrifugation at 14 k RPM and removal of the supernatant for cytokine analysis. Calibration controls and recombinant standards were used to generate standard curves as specified by the manufacturer. Linear (Pearson) correlation coefficients were calculated using InStat™ software (GraphPad Software Inc., San Diego, Calif.). Spearman rank correlations were calculated whenever Gaussian assumptions were not appropriate, as indicated in the text. To test the hypothesis that individual cytokines were upregulated in early RA over two control groups, cytokine concentrations were plotted and p values calculated by Kruskal-Wallis tests with Dunn's multiple comparisons using Prism™ software (GraphPad Software, Inc.) (FIG. 8)

### Results

We have tested whether autoantibody and cytokine profiles are superior markers for the diagnosis and prediction of rheumatoid arthritis. We have demonstrated herein that autoantibody profiling by antigen microarray analysis (FIGS. 1 and 2), allowing the simultaneous detection of autoantibodies directed against 225 distinct candidate RA autoantigens. Antigen arrays demonstrated differential targeting of citrullinated vs. native epitopes in subsets of RA patients (FIGS. 3 and 4), and that targeting of citrullinated epitopes was associated with elevations in the inflammatory marker C-reactive protein (CRP) (FIG. 4). We also performed multiplex cytokine analysis with a bead array based on the 22-Plex Beadlyte Kit and Luminex xMAP platform, and demonstrated that approximately ⅓ of RA patients have significant elevations of cytokines in blood and that cytokine elevations were associated with laboratory parameters predictive for the development of more severe disease (FIG. 7).

We further demonstrated that subgroups of patients with more severe disease could be identified based on distinct profiles of array-determined autoantibody reactivities and elevated blood cytokine profiles: patients who possessed autoantibody responses that targeted panels of citrullinated antigens and elevated serum cytokines were more likely to have active and more severe disease, whereas patients with immune responses that targeted panels of native, unmodified polypeptides and low or undetectable serum cytokines were likely to have less active, mild disease (FIGS. 4 and 7). Knowledge that an individual patient is likely to develop more severe disease would warrant more aggressive therapy, with both biological (anti-TNF, anti-IL-1, anti-IL-6, etc) and small molecule (methotrexate, leuflonamide, hydroxychloriquine, cyclosporine, etc.) agents, to minimize disease activity and thereby reduce the level of future joint inflammation and destruction.

### Example 2

### Methods for Predicting Response to Anti-TNF Therapy

### Blood Autoantibody and Cytokine Profiles in Pre-Treatment Samples Predict Etanercept Responder and Non-Responder Rheumatoid Arthritis Patient Subgroups

Rheumatoid arthritis is an inflammatory synovitis affecting 0.6%-1% of the World population. Treatment of RA with the anti-TNF alpha antagonists etanercept, infliximab and adalimumab produces significant clinical benefit in approximately ⅓ of patients, mild clinical benefit in ⅓ and little to no clinical benefit in ⅓ of patients based on American College of Rheumatology (ACR) response criteria (Genovese et al, 2002, Arthritis and Rheumatism, 46:1443-50). To date, in routine clinical practice the responsiveness of individual patients to anti-TNF alpha therapy is determined via an empiric therapeutic trial with etanercept, infliximab or adalimumab for approximately 1-12 months to determine if the patient experiences significant clinical improvement based on patient self-assessment and physician assessment.

We tested whether patients with RA can be stratified as Responders (R) and Non-Responders (NR) to specific therapy, based on distinct autoantibody and cytokine profiles obtained before the respective treatment was started. We analyzed serum samples from a group of 29 patients with RA according to the revised American College of Rheumatology (ACR) classification criteria of 1985. Blood samples where obtained before, and three months after treatment with a TNF alpha blocking agent (etanercept) was started.

After 3 months of therapy with the TNF alpha blocker etanercept, patients were classified into Responder (R) and Non-Responder (NR) groups based on the ACR response criteria, an international composite index for RA widely used to measure response to therapy in clinical trials. Arthritis arrays were used to determine autoantibody profiles in blood samples derived from RA patients prior to treatment with the anti-TNFα therapy. Patients were treated with etanercept and after 12 weeks their Responder status was determined based on the American College of Rheumatology response criteria for RA. In FIG. 9A, Responders (R) demonstrated ACR50 or greater responses to etanercept, while Non-Responders (NR) exhibited ACR20 or lower responses to etanercept therapy. The Significance Analysis of Microarrays (SAM) algorithm was applied to identify antibody reactivities (FDR for individual antigens was set at <0.03 for both experiments A and B, with significant differences between NR and R. Responders demonstrated increased autoantibody targeting of multiple citrullinated and native epitopes at baseline (prior to etanercept therapy). The images represent hierarchical clustering of patients and antigen features. The tree dendrograms represent the relationships between patient samples or antigen features, with branch lengths representing the extent of similarities in array reactivity determined by the cluster algorithm. FIG. 9B presents data from an experiment characterizing a larger number of patients from the same cohort of patients, comparing baseline autoantibody profiles in etanercept Non-Responders (NR; ACR20 or worse response) with Responders (R; ACR40 or better response). The magnitude of the ACR response (e.g. ACR50, etc) is indicated for Responders in FIG. 9B.

FIGS. 9A and B both demonstrate increased autoantibody targeting of a variety of citrullinated and native epitopes in baseline (pre-treatment) samples in etanercept Responders as compared to Non-Responders. Antigen epitopes in the signature included epitopes derived from citrullinated filaggrin, native and citrullinated vimentin, native and citrullinated fibrinogen, native and citrullinated fibromodulin, COMP, biglycan, clusterin, lumican, Histone H2B, and synthetic cyclic citrullinated peptides (CCP). The tree dendrograms in FIGS. 9A and B represent the relationships among patient samples or antigen features, with branch lengths representing the extent of similarities in array reactivity determined by the cluster algorithm.

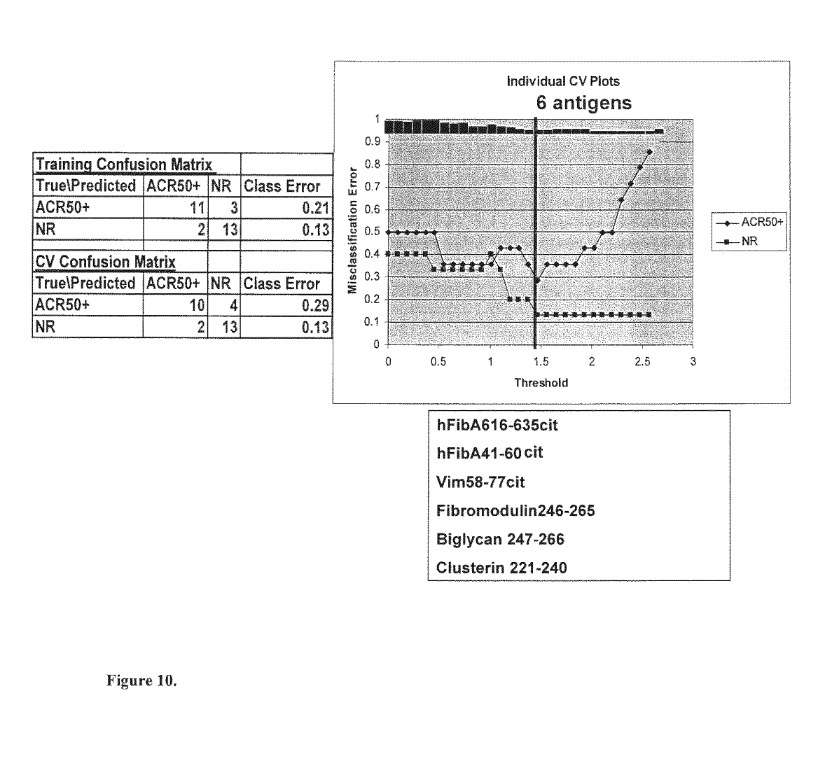

In FIG. 10, Prediction Analysis of Microarrays (PAM) was applied to identify autoantibody specificities in pre-treatment samples that are predictive for subsequent response to anti-TNF therapy with etanercept. PAM identified 6 peptides, including 3 citrulline-substituted peptides (human fibrinogen A 616-635-citrulline, human fibrinogen A 41-60-citrulline and vimentin 58-77-citrulline) and 3 native peptides (fibromodulin 246-266, biglycan 247-266 and clusterin 221-240), against which autoantibodies were predictive for etanercept responsiveness. In the particular cohort analyzed, in re-randomized samples (confusion matrix) PAM correctly classified 10 of 14 (71%) of the ACR50 or better Responders as well as 13 of 15 (87%) of the ACR20 or worse Non-Responders.

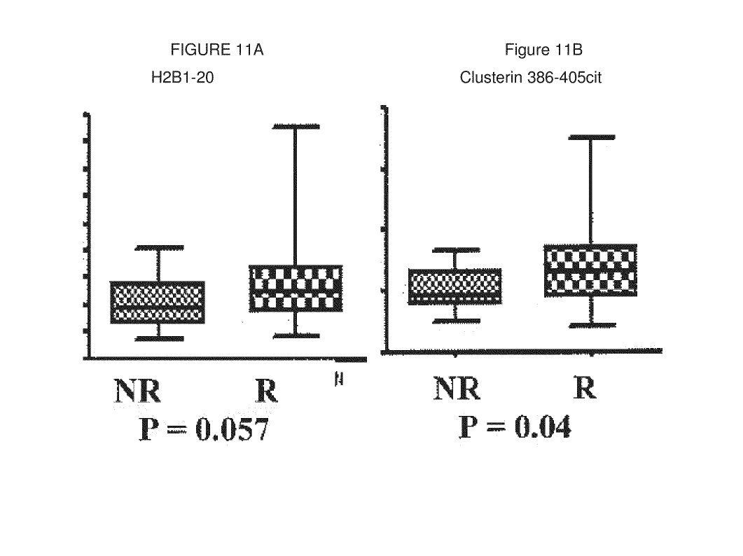

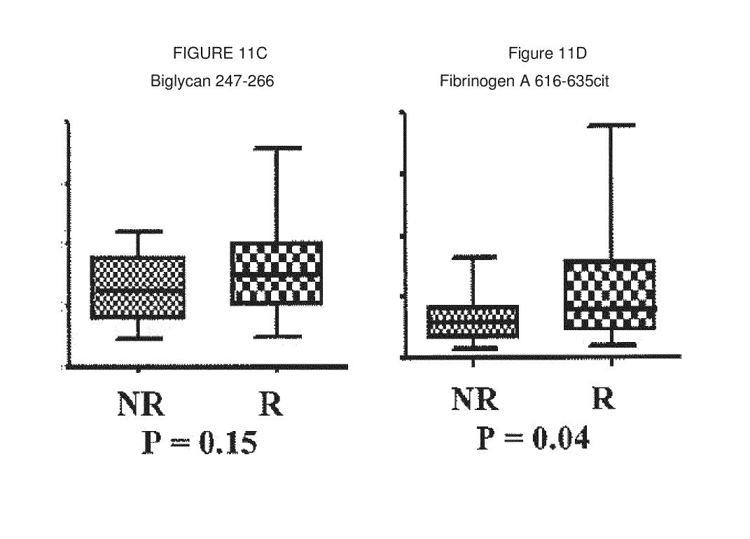

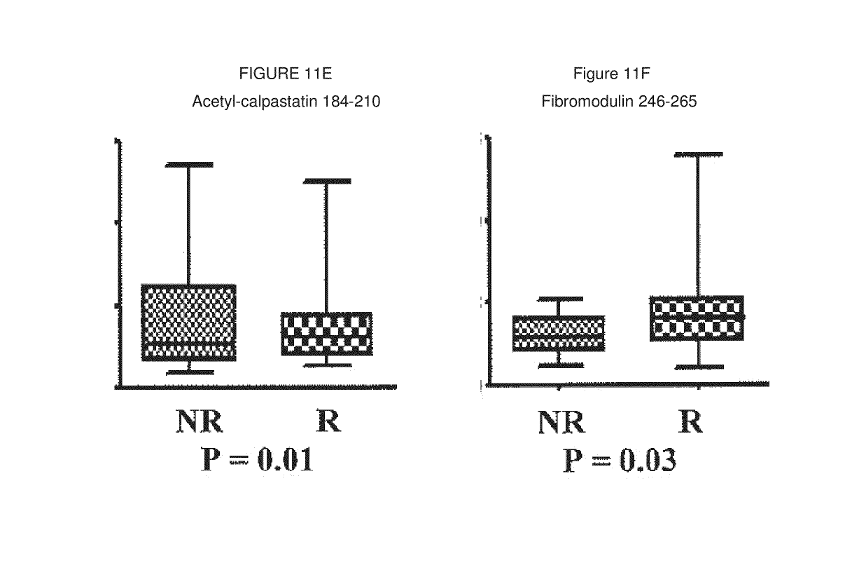

Enzyme-linked immunosorbent assay (ELISA) analyses were performed to confirm elevated autoantibodies targeting a select set of the SAM (FIG. 9) and PAM (FIG. 10) identified “peptide hits” (targets of the autoantibody responses) in pre-treatment sera derived from etanercept Responders as compared to Non-Responders (FIG. 11). Pre-treatment samples were analyzed for autoantibody targeting of 20 peptide antigens: acetyl-calpastatin 184-210, biglycan 247-266, fibromodulin 103-122, fibromodulin 246-265, H2B 1-20, fibrinogenA 41-60-cit, fibrinogenA 616-635-cit, fibrinogenB 421-440, osteoglycin 176-196, lumican 198-217, PG4 1184-1203, serine protease-II 461-480, Tenascin-C 122-141-cit, vimentin 58-77-cit, ApoE 277-296, clusterin 386-405-cit, H2A 95-114, HSP58 peptide, vimentin 436-455-cit, and cfc1-cyc2. Statistically, the most significant differences in autoantibody reactivity between Responders and Non-Responders at the pre-treatment time point were against the following peptide antigens: acetyl-calpastatin 184-210, clusterin 386-405-cit, fibrinogenA 616-635-cit, and fibromodulin 246-265. While p-values indicated in FIG. 11 reflect simple t-testing (ABNOVA), regression analysis revealed that 9 out of the 20 peptides tested were differentially targeted in Responders versus Non-Responders with p-values <0.05. Specifically, these antigen peptides comprised: acetyl-calpastatin peptide, ApoE 277-296-cit, fibromodulin 246-265, PG4 1184-2003, fibrinogenA 616-635-cit, serine protease II 433-452, clusterin 386-405-cit, H2B 1-20 and HSP58 peptide.

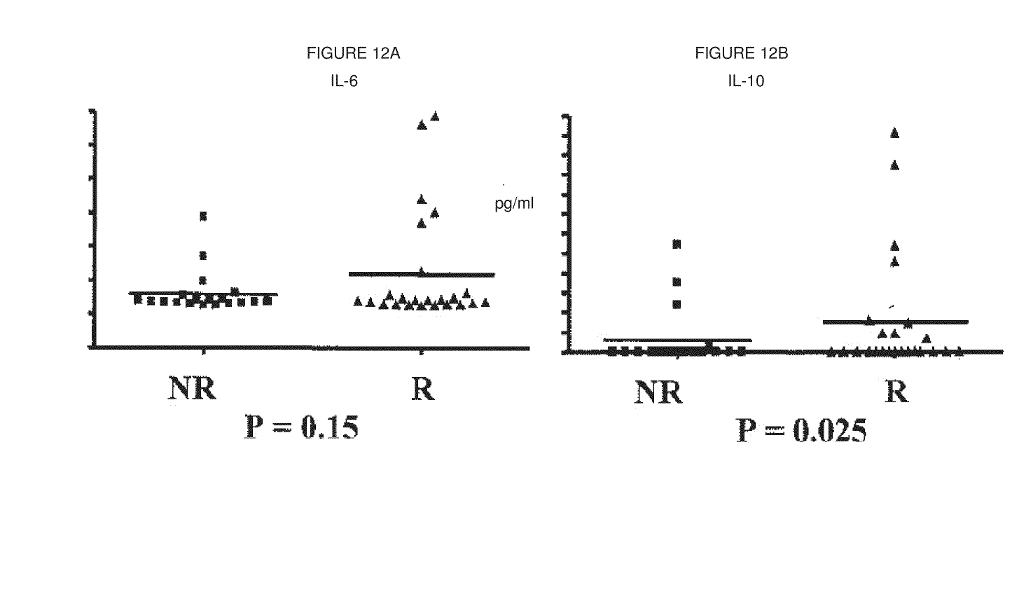

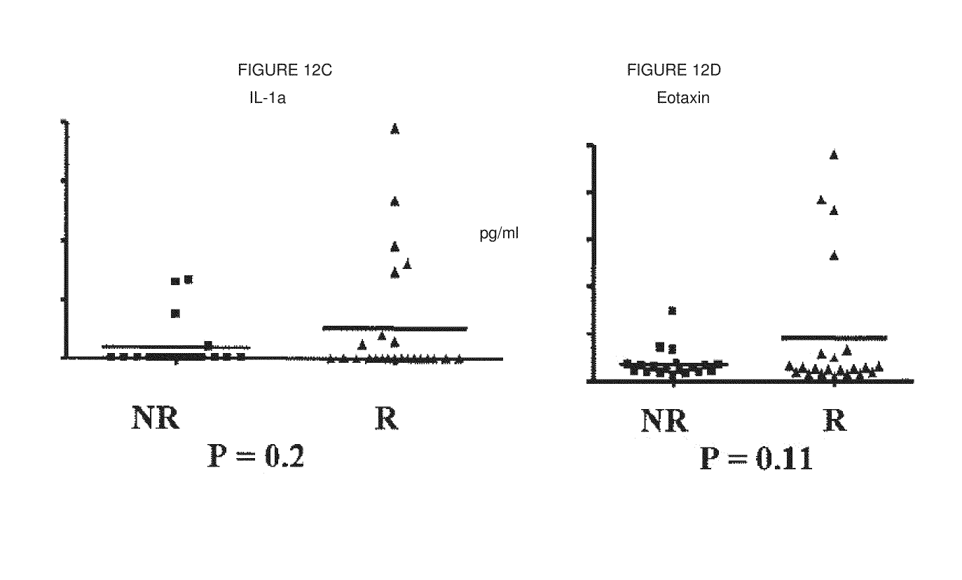

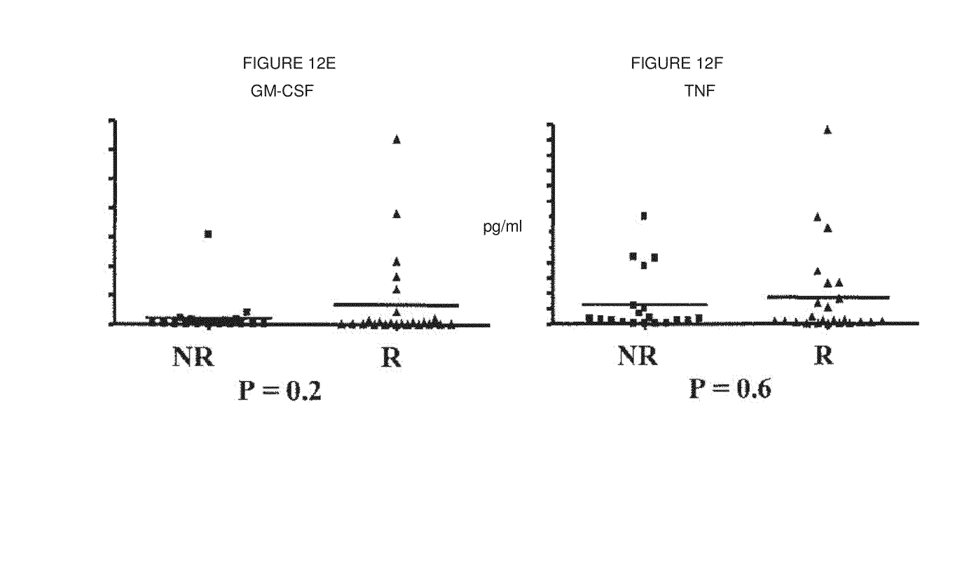

FIG. 12 demonstrates that elevated cytokine levels are also present in the blood of a subset of etanercept Responders (ACR50 or greater) as compared to Non-Responders (ACR20 or worse). Overall, the subpopulations of etanercept Responders with elevated blood cytokines levels were smaller (FIG. 12) than the subpopulations exhibiting increased autoantibodies (FIG. 9). We applied multi-dimensional scaling (MDS) to demonstrate that combining autoantibody and cytokine profiles provide enhanced predictive value for identifying patients likely to respond to anti-TNF therapy.

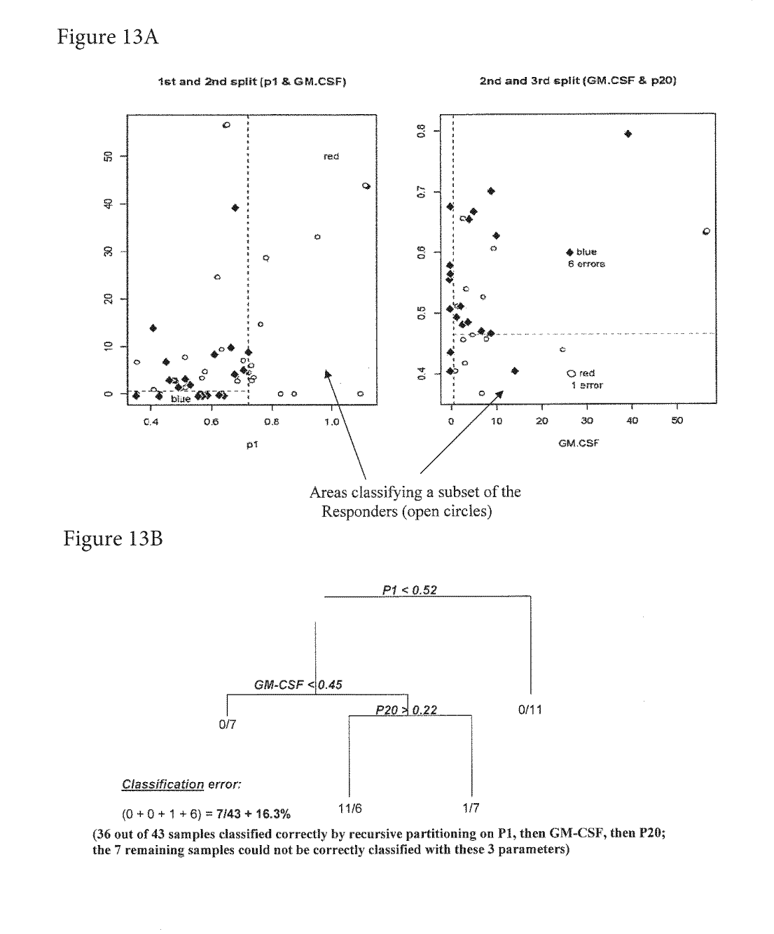

Multi-dimensional scaling (MDS) analysis identified blood autoantibody and cytokine biomarkers with utility as predictors of response to etanercept therapy. In analyses using the cohort of 43 etanercept-treated patients (FIG. 9), differential serum expression of a total of 14 cytokines and chemokines (TNF, IL-1α, IL-4, IL-6, IL-10, IL-12p40, IL-12p70, IL-15, GM-CSF, eotaxin, IP-10, MCP-1, flt3-ligand, FGF-2) was measured by a multiplex bead assay using the methods described for FIG. 7. Using a MDS classification algorithm, a combined signature profile of three biomarkers consisting of the chemokine GM-CSF and the autoantigen peptides osteoglycin 277-296 and acetyl-calpastatin 184-210 was generated that provided the best classification of 43 samples in Responders and Non-Responders, with a total classification error of 7/43 (16.3%) (FIG. 13).

Recursive partitioning (RP) can be applied to segregate etanercept Responders and Non-Responders based on different levels of blood antibodies and cytokines, and specifically use lower levels to classify the subset of samples which are unable to be classified using higher levels of autoantibodies and cytokines. Using a combination of markers consisting of just one category of molecules measured by one diagnostic assay, for example the cytokines/chemokines IL-12p70 and eotaxin, also enable a prediction of response, albeit with a larger classification error (26%). Due to substantial correlations between biomarkers of just one category, decision trees based on these markers may be unstable with respect to small perturbations, and a number of trees can thus give rise to a similar classification performance. The best-performing biomarkers of different categories (i.e. cytokines and autoantibodies) that are measured and identified on different assaying platforms may desirably be combined for enhanced classification.

In summary, multiplex autoantibody and cytokine analysis provides a means to identify blood antibody and/or cytokine signatures that distinguish pre-treatment RA patients likely to exhibit a significant clinical response to etanercept anti-TNF treatment versus RA patients likely to not exhibit a significant response. Antibody and cytokine profiling can be applied to identify individual patients likely to respond to, or to not response to, therapy with an anti-TNF alpha drug or other DMARDs, and thus can guide identification and selection of (1) early arthritis or early RA patients whom would likely benefit from DMARD therapy, and (2) early arthritis, early RA, or established RA patients with an increased likelihood of responding to etanercept or another DMARD therapy.

### Example 3

### Use of Multiple Technology Platforms, RA Patient Sample Sets and Statistical Analysis Algorithms to Identify Autoantibody Specificity Profiles Specific for Etanercept Responders Versus Non-Responders

Example 2 illustrates that the analysis of autoantibody and cytokine expression patterns in blood using a variety of proteomics technologies and biostatistics tools can be complementary and may facilitate discovery of multi-molecule biosignatures of lesser complexity, while predicting clinical outcomes such as response to therapy with accuracy comparable to larger-scale signatures. The more variables that are used, the higher the classification rate, but also the tendency to overfit the data. Thus, the classification rate in these examples can represent an overestimation of the prediction rate in an independent second cohort. However, combination of markers from different assay platforms can demonstrate superior performance as compared with combination of markers from just one assay platform, as evidenced by the examples in FIG. 13 and Table 4. As a consequence of this observation, adding top-performing biomarkers measured by additional, unrelated diagnostic assays may provide greater predictive power with respect to prediction of clinical outcome in independent cohorts. Application of a number of modeling techniques such as predictive models and neuronal networks, may further improve the accuracy of predictions based on smaller panels of biomarkers.

Table 4 illustrates use of multiple technology platforms (synovial antigen arrays, ELISA), multiple sample cohorts, and multiple statistical approaches to identify consensus autoantibody reactivities in pre-treatment samples with the greatest and most consistent predictive value for identifying etanercept Responders (ACR50 or greater response) from Non-Responders (ACR20 or worse response). Identification of these consensus autoantibody reactivities is based on antibody analysis results from two different technology platforms, including synovial antigen microarrays (FIG. 1) and peptide ELISA (FIG. 11). It also utilizes results from multiple independent cohorts of RA patients treated with the anti-TNF agent etanercept (Cohort 1 and Cohort 2). Further, multiple analysis algorithms and strategies, including SAM, PAM, hieratical clustering and regression analyses, were utilized to identify consensus reactive peptides that are most consistently reactive in pre-treatment samples from both cohorts of etanercept treated patients using multiple different autoantibody detection methods. Based on these analyses, Table 4 presents 14 autoantibody specificities with favorable performance characteristics for predicting response to anti-TN F etanercept therapy.

Identification of autoantibody and cytokine biomarker profiles for identifying: (1) early arthritis or early RA patients with progressive disease and thus most likely to benefit from DMARD therapy, and (2) early arthritis, early RA, or established RA patients most likely to response to a particular therapeutic agent, can be based on a systematic process of discovering, confirming and validating candidate biomarkers. Such an approach can involve using synovial microarrays to profiling autoantibodies and Luminex bead arrays to profile cytokines to discover autoantibodies and cytokines predictive for response to a particular therapeutic agent, for example CTLA4-Ig, in one cohort of CTLA4-Ig-treated RA patients. These results are then compared with results on a separate independent cohort of CTLA4-Ig treated RA patients, and consensus autoantibody reactivities and elevated cytokines identified. A third independent cohort of CTLA4-Ig-treated patients and additional proteomic technologies (for example ELISA) are used to further validate the identified autoantibody and cytokine biomarkers for classifying patients most likely to experience significant clinical benefit from treatment with CTLA4-Ig.

In addition to accounting and compensating for variances between sample cohorts and technologies, the combination of biomarkers needed for optimal prediction may vary for different therapeutic agents and/or clinical endpoints, e.g. in predicting a response to IL-6RA; CTLA4-Ig; rituximab, methotrexate, etc. Predictive biomarker signatures may also differ between women and men, or for patients belonging to different age groups.

TABLE 4
Identification of consensus autoantibody specificities in pre-treatment
samples predictive for etanercept responsiveness in RA patients.

| | | | | | Cohort 2 | |
|---|---|---|---|---|---|---|
| | Cohort 1 | Cohort 2 | Combination | Cohort 2 | regression | PAM |
| | array | array | cluster | ELISA | analysis | hit |
| Serine Protease II 461-480cit | Yes | yes | yes | p = 0.14 | | |
| hFibB421-440 | Yes | yes | yes | p = 0.17 | | yes |
| Biglycan | Yes | no | no | p = 0.15 | | |
| acetyl-calpastatin | Yes | no (y) | no | p = 0.01 | p < 0.05 | |
| vimentin 436-455cit | o/l pep | yes | yes | p = 0.057 | | yes |
| clusterin 386-405cit | Yes | yes | no | p = 0.04 | p < 0.05 | |
| hFibA211-230cit | Yes | no (y) | yes | p = 0.6 | n.d. | |
| Fibromodulin 246-265 | o/l pep | yes | no | p = 0.03 | p < 0.05 | |
| hFibA616-635cit | Yes | yes | no | p = 0.04 | p < 0.05 | yes |
| H2B1-20 | Yes | no (y) | no | p = 0.057 | p < 0.05 | yes |
| osteoglycin 177-196 | No | yes | no | n.d. | p < 0.05 | |
| PG4 1184-1203 | Yes | no | no | p = 0.10 | p < 0.05 | |
| ApoE277-296cit | Yes | no | no | p = 0.15 | p < 0.05 | |
| cfc48-65cit | Yes | no | no | p = 0.8 | n.d. | yes |

o/l pep means reactive in an overlapping peptide epitope
(y) means present in a cluster of lower statistical significance

### Example 4

### Use of Proteomic Biomarkers to Guide Initiation of Therapy in Early Arthritis

The value of combination therapy in controlling signs, symptoms and radiographic progression of RA was established by several studies, particularly the combination of methotrexate with a biological agent such as a TNF blocker in reducing disease activity. While current treatment approaches can produce benefits in patients with early undifferentiated arthritis and/or early RA, the methods of the invention find use in selecting patients for targeting therapy more selectively and to determine which patients respond best to various agents or combinations. In the following example, the use of multi parameter biomarker assays to determine signature patterns is provided in the context of a clinical trial aimed at improving targeting of effective therapies to subsets of patients with early RA, while comparing different treatments.

In a prospective clinical trial in early rheumatoid arthritis patients, the study group compares a biological DMARD such IL-6-RA (IL-6 receptor antagonist) or rituximab or CTLA4-Ig, alone and in combination with conventional DMARDs including methotrexate, to delay progression of the disease and prevent long-term disability. Primary and secondary endpoints are chosen as i.e. the COBRA or BeST trials, namely improvements in disability (Health Assessment Questionnaire, HAQ) scores, radiographic scores, and DAS28 (disease activity score 28). Inclusion criteria in the study recruitment phase include presence of a specific biomarker signature pattern as provided in the methods of the invention to predict response to the respective biological drug. Such biomarker signature pattern may include several autoantibody reactivities such as anti-acetyl-calpastatin peptide, anti Fibrinogen A peptide, anti-H2B peptide, anti-fibromodulin peptide and anti-citrullinated peptide reactivity (or other antigens as outlined in Tables 1 and 4), as well as elevated levels of proinflammatory cytokine IL-12, eotaxin and GM-CSF (and other cytokines outlined in Table 2).

Other pre-established biomarker signatures targeted to predict response to methotrexate or any other of the conventional DMARDs such as plaquenil, leflunomide, prednisone, etc. may or may not be part of the study protocol, and may or may not be part of inclusion criteria. These signature patterns may be overlapping with signature patterns associated with prediction of response to the primary biological DMARD studied, or another conventional DMARD, or may be different. Hence, individuals who do not demonstrate the specific biomarker signature, and consequently have a higher chance to respond poorly or not at all to the study drug, will not meet inclusion criteria and may be excluded from the study. Patients who exhibit the specific biomarker signature pattern will meet inclusion criteria and have substantially reduced risk of being treated with an ineffective drug. Such study design will substantially reduce drop-out rates due to drug inefficacy, and thus increase quality of patient care in clinical trials.

Anticipated benefits: Response to therapy, i.e. anti-TNF agents, CTLA4-Ig, rituximab, IL-6RA (and/or any of the other drugs used in the control treatment arms of a clinical trial) are predicted from biomarker signature patterns determined in baseline serum samples by a pre-configured multiparameter assay, designed to measure specific autoantibodies and cytokines identified to be associated with response or lack of response to individual biological and/or conventional DMARDs.

Determination of pre-specified biomarker signature patterns measured in serum or plasma drawn at the recruitment visit of a prospective study participant leads to more selective enrolment of patients into the particular clinical trial that will benefit the individual patient with early RA the most. Additional data is collected during the course of the study by comparing a pre-defined pre-treatment signature pattern with a post-treatment signature pattern of the same biomarker panel, to identify surrogates for response to therapy. For example, a drop in a combination of autoantibody titers, inflammatory markers and cytokine concentrations (antibodies against RF, cit fibrinogenA peptides, cit filaggrin peptides, H2B peptides, anti-vimentin peptides; CRP and IL-6 concentrations) at 0.5, 1, 3 and/or 6 months of therapy enables a measure of response. Ratios of pre-treatment and post-treatment titers/concentrations are used to calculate scores that enable improved quantification of response to therapy, in intervals as determined by the study protocol. An unchanged signature pattern indicates failure to respond, and such information, together with clinical and other surrogates of response (or disease activity) such as radiographic scores, HAQ scores and DAS28 scores allows the development of improved clinical decision trees to determine the need for a patient to receive a different therapy. Improved care for patients with early RA included in such clinical trials, and subsequently for patients with RA seen in clinical practice, utilizes development and implementation of powerful multi-parameter bioassays, as described in this invention.

### Example 5

### Determination of Autoantibody and Cytokine Profiles that Identify Autoimmune Disease Patients that Will Respond Versus Not-Respond to Biological and Non-Biological Therapies

Multiplex analyses of blood and other biological fluids for cytokines, antibodies and other protein markers are performed on samples derived from patients with a variety of autoimmune diseases to identify profiles with clinical utility for discriminating treatment Responder from Non-Responder patients. Such autoimmune diseases include rheumatoid arthritis, psoriatic arthritis, ankylosing spondylitis, systemic lupus, juvenile rheumatoid arthritis, adult Still's disease, Reiter's syndrome, multiple sclerosis, autoimmune diabetes, psoriasis, myasthenia gravis, bullous skin diseases, vasculitides, autoimmune thyroid diseases, inflammatory bowel diseases, autoimmune peripheral neuropathies, and others.

To determine autoantibody and cytokine profiles with utility for guiding the selection of a therapeutic agent to which the patient is more likely to respond, the blood or other biological fluid is obtained from the autoimmune disease patient prior to and then 1, 3 and/or 6 months following initiation of a biological therapy (recombinant antibody, cytokine or other protein therapy; for example etanercept, adalimumab, rituximab, IL-6RA, CTLA4-Ig, interferon beta, etc.) or non-biological therapy (small molecules, such as methotrexate, cyclosporine, cellcept, cytoxan, plaquenil, sulfasalazine, leflunomide, etc). Patients are determined to be Responders or Non-Responders based on clinical response criteria, such as the American College of Rheumatology (ACR) response criteria (for RA), Crohn's disease activity index (CDAI) (for Crohn's disease), MRI evaluation of brain lesions (for MS), or laboratory markers such as blood glucose or hemoglobin A1C (for autoimmune diabetes). The blood or other biological fluid is analyzed for the specificity of the antibodies present using antigen arrays or another assay for measuring antibody specificity, cytokines and chemokines are characterized using a bead-array assay or anther suitable assay, and other protein makers can be measured using ELISA, a bead-array or another proteomics assay. Statistical methods, for example SAM and PAM, are then applied to identify pre-treatment autoantibody, cytokine and other protein biomarkers associated with likelihood for individual patients to respond to therapy with a particular biological or non-biological agent (in an analogous fashion to that described for etanercept-Responder and Non-Responder RA patients in Example 2 and FIGS. 9 and 10).

Autoantibody, cytokine and other proteomic profiling assays are also applied in combination with statistical methods to identify biomarkers profiles in blood, spinal fluid or other body fluids that are associated with a positive or successful response to therapy (in an analogous fashion to that described for monitoring response to etanercept therapy in RA Responder patients in Examples 2 and 3, and FIGS. 9-13). Such profiles are developed for a variety of autoimmune diseases and a variety of biological and small molecule therapies. The baseline level can be established using a level of autoantibodies, cytokines and other proteins that are determined for diagnostic purposes, or a level obtained at a later time point. A treatment is then administered, e.g., one or more doses of a treatment, and the level of autoantibodies, cytokines and proteins is determined again. A decrease in the level of autoantibodies will generally indicate that the treatment is effective, while no change or an increase will generally indicate that the treatment is ineffective or harmful. Quantitative and semi-quantitative methods for determining the amount of autoantibodies per volume of blood are known in the art. Statistical algorithms such as SAM, PAM, multidimensional scaling, recursive partitioning, principle components alanalysis, predictive algorithms and neural networks can be applied to identify autoantibody, cytokine and protein profiles that are associated with a positive response to therapy. Identification of profiles that enable clinician monitoring of response to therapy would be used to guide the clinician to continue treatment with a particular biological or small molecule, or switch to a different therapeutic agent.

### Example 6

### Methods of Monitoring Response to Anti-TNF Therapy

### Decreases in Blood Autoantibodies and Cytokines Reflect a Positive Clinical Response to Anti-TNF Therapy

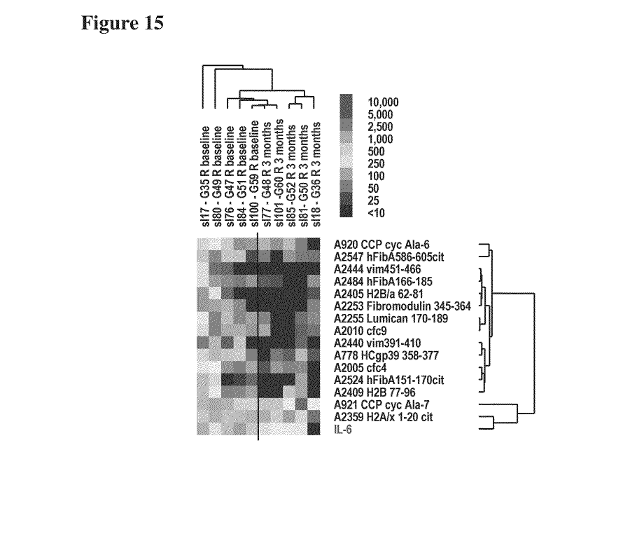

Antibody and cytokine profiles are utilized to monitor response to an anti-TNF therapy. We tested whether there was a reduction in blood (serum) autoantibody reactivity and cytokine levels associated with successful clinical response to etanercept in patients with RA. RA patients were stratified into Responders (R) and Non-Responders (NR) to etanercept therapy, based on the ACR response criteria. Arthritis arrays and bead array cytokine profiling were used to determine autoantibody profiles in blood samples derived from RA etanercept-Responder patients prior to treatment with etanercept and after 12 weeks of etanercept therapy. In FIG. 15, using the same cohort of patients treated with TNF-blocking therapy described in FIG. 9A, we observed a profile of blood autoantibodies and cytokines, whereby significant decline in reactivity was associated with the subgroup of patients who demonstrated an ACR 50 response to etanercept therapy (FIG. 15). Autoantibody and cytokine analysis is thus a tool to identify patients that responded to anti-TNF therapy. No changes in blood autoantibody and/or cytokine levels were observed in RA patients that did not exhibit a significant clinical response to anti-TNF therapy (>ACR20 response). Antibody and cytokine profiling are thus applied to identify individual patients who are Responders, and can guide clinicians to continue use of anti-TNF therapy or other DMARD therapy.

What is claimed is:

1. A method for generating quantitative data for a subject comprising:
performing at least one immunoassay on a first sample from the subject to generate a first dataset comprising the quantitative data, wherein the quantitative data represents at least three protein markers wherein the at least three protein markers comprise three or more of IL-1α, IL-6, TNFα, IL-12p40, IP-10/CXCL10, Eotaxin/CCL11, MCP-1/CCL2, or IL-8/XCXL8; and
wherein the first subject has psoriatic arthritis (PsA) or is suspected of having PsA.

2. The method of claim 1, wherein the at least three protein markers comprise IL-1α, IL-6, TNFα, IL-12p40, IP-10/CXCL10, Eotaxin/CCL11, MCP-1/CCL2, and IL-8/XCXL8.

3. The method of claim 1, wherein performance of the at least one immunoassay comprises:
obtaining the first sample, wherein the first sample comprises the protein markers;
contacting the first sample with a plurality of distinct reagents;
generating a plurality of distinct complexes between the reagents and markers; and
detecting the complexes to generate the data.

4. The method of claim 1, wherein the at least one immunoassay comprises a multiplex assay.


SEQUENCE LISTING (83 sequences)

SEQ ID NO: 1 - protein, 20 aa, Homo sapiens
  VARIANT 1, 4, 11: Xaa = citrulline
XLLXKGHYAEXVGAGAPVYL

SEQ ID NO: 2 - protein, 20 aa, Homo sapiens
  VARIANT 10, 16, 20: citrulline
ILELAGNAAXDNKKTXIIPX

SEQ ID NO: 3 - protein, 20 aa, Homo sapiens
MSGRGKQGGKARAKAKTRSS

SEQ ID NO: 4 - protein, 20 aa, Homo sapiens
  VARIANT 4, 12, 18: citrulline
MSGXGKQGGKAXAKAKTXSS

SEQ ID NO: 5 - protein, 20 aa, Homo sapiens
MSGRGKTGGKARAKAKSRSS

SEQ ID NO: 6 - protein, 20 aa, Homo sapiens
  VARIANT 4, 12, 18: citrulline
MSGXGKTGGKAXAKAKSXSS

SEQ ID NO: 7 - protein, 20 aa, Homo sapiens
RLLRKGHYAERVGAGAPVYL

SEQ ID NO: 8 - protein, 20 aa, Homo sapiens
RLLRKGNYAERVGAGAPVYL

SEQ ID NO: 9 - protein, 20 aa, Homo sapiens
MPEPSKSAPAPKKGSKKAIT

SEQ ID NO: 10 - protein, 20 aa, Homo sapiens
KKAVTKAQKKDGKKRKRSRK

SEQ ID NO: 11 - protein, 20 aa, Homo sapiens
MPEPVKSAPVPKKGSKKAIN

SEQ ID NO: 12 - protein, 20 aa, Homo sapiens
EASRLAHYNKRSTITSREIQ

SEQ ID NO: 13 - protein, 21 aa, Homo sapiens
GGGVRGPRVVERHQSACKSDS

SEQ ID NO: 14 - protein, 20 aa, Homo sapiens
NYKCPSGCRMKGLIDEVNQD

SEQ ID NO: 15 - protein, 20 aa, Homo sapiens
DLLPSRDRQHLPLIKMKPVP

SEQ ID NO: 16 - protein, 20 aa, Homo sapiens
  VARIANT 3, 9, 15, 17: citrulline
NNXDNTYNXVSEDLXSXIEV

SEQ ID NO: 17 - protein, 20 aa, Homo sapiens
  VARIANT 6, 8: citrulline
DLLPSXDXQHLPLIKMKPVP

SEQ ID NO: 18 - protein, 20 aa, Homo sapiens
  VARIANT 3: citrulline
PSXGKSSSYSKQFTSSTSYN

SEQ ID NO: 19 - protein, 20 aa, Homo sapiens
MKRLEVDIDIKIRSCRGSCS

SEQ ID NO: 20 - protein, 20 aa, Homo sapiens
MKPVPDLVPGNFKSQLQKVP

SEQ ID NO: 21 - protein, 20 aa, Homo sapiens
TKTVIGPDGHKEVTKEVVTS

SEQ ID NO: 22 - protein, 20 aa, Homo sapiens
HRHPDEAAFFDTASTGKTFP

SEQ ID NO: 23 - protein, 20 aa, Homo sapiens
STSYNRGDSTFESKSYKMAD

SEQ ID NO: 24 - protein, 20 aa, Homo sapiens
  VARIANT 5, 8, 12: citrulline
GGGVXGPXVVEXHQSACKDS

SEQ ID NO: 25 - protein, 20 aa, Homo sapiens
  VARIANT 5, 8, 12: citrulline
GGGVXGPXVVEXHQSACKDS

SEQ ID NO: 26 - protein, 20 aa, Homo sapiens
  VARIANT 3, 8, 16: citrulline
QMXMELEXPGGNEITXGGST

SEQ ID NO: 27 - protein, 20 aa, Homo sapiens
  VARIANT 2: citrulline
PXNPSSAGSWNSGSSGPGST

SEQ ID NO: 28 - protein, 20 aa, Homo sapiens
  VARIANT 6: citrulline
STSYNXGDSTFESKSYKMAD

SEQ ID NO: 29 - protein, 20 aa, Homo sapiens
  VARIANT 6, 12, 15: citrulline
THSTKXGHAKSXPVXGIHTS

SEQ ID NO: 30 - protein, 20 aa, Homo sapiens
KHLLLLLLCVFLVKSQGVND

SEQ ID NO: 31 - protein, 20 aa, Homo sapiens
  VARIANT 2, 8, 15: citrulline
HXPLDKKXEEAPSLXPAPPP

SEQ ID NO: 32 - protein, 20 aa, Homo sapiens
PAPPPISGGGYRARPAKAAA

SEQ ID NO: 33 - protein, 20 aa, Homo sapiens
PCTVSCNIPVVSGKECEEII

SEQ ID NO: 34 - protein, 20 aa, Homo sapiens
QYTWDMAKHGTDDGVVWMNW

SEQ ID NO: 35 - protein, 20 aa, Homo sapiens
VWMNWKGSWYSMRKMSMKIR

SEQ ID NO: 36 - protein, 20 aa, Homo sapiens
PAPPPISGGGYRARPAKAAA

SEQ ID NO: 37 - protein, 20 aa, Homo sapiens
LKDLWQKRQKQVKDNENVVN

SEQ ID NO: 38 - protein, 20 aa, Homo sapiens
QGFGNVATNTDGKNYCGLPG

SEQ ID NO: 39 - protein, 20 aa, Homo sapiens
QKDSDGDGIGDACDNCPQKS

SEQ ID NO: 40 - protein, 20 aa, Homo sapiens
HNKIQAIELEDLLRYSKLYR

SEQ ID NO: 41 - protein, 20 aa, Homo sapiens
EDLLRYSKLYRLGLGHNQIR

SEQ ID NO: 42 - protein, 20 aa, Homo sapiens
LQVVRLDGNEIKRSAMPADA

SEQ ID NO: 43 - protein, 20 aa, Homo sapiens
  VARIANT 5: citrulline
NQISXVPNNALEGLENLTAL

SEQ ID NO: 44 - protein, 20 aa, Homo sapiens
  VARIANT 18: citrulline
SFCTVVDVVNFSKLQVVXLD

SEQ ID NO: 45 - protein, 20 aa, Homo sapiens
NQISRVPNNALEGLENLTAL

SEQ ID NO: 46 - protein, 20 aa, Homo sapiens
  VARIANT 5: citrulline
NQISXVPNNALEGLENLTAL

SEQ ID NO: 47 - protein, 20 aa, Homo sapiens
NLTALYLQHDEIQEVGSSMR

SEQ ID NO: 48 - protein, 20 aa, Homo sapiens
  VARIANT 20: citrulline
NLTALYLQHDEIQEVGSSMX

SEQ ID NO: 49 - protein, 20 aa, Homo sapiens
GSSMRGLRSLILLDLSYNHL

SEQ ID NO: 50 - protein, 20 aa, Homo sapiens
  VARIANT 4: citrulline
VPSXMKYVYFQNNQITSIQE

SEQ ID NO: 51 - protein, 20 aa, Homo sapiens
LEQLYMEHNNVYTVPDSYFR

SEQ ID NO: 52 - protein, 20 aa, Homo sapiens
RLKEDAVSAAFKGLKSLEYL

SEQ ID NO: 53 - protein, 20 aa, Homo sapiens
RLPSGLPVSLLTLYLDNNKI

SEQ ID NO: 54 - protein, 20 aa, Homo sapiens
PVEVSRKNPKFMETVAEKAL

SEQ ID NO: 55 - protein, 20 aa, Homo sapiens
QTHMLDVMQDHFSRASSIID

SEQ ID NO: 56 - protein, 20 aa, Homo sapiens
  VARIANT 3, 6: citrulline
AEXLTXKYNELLKSYQWKML

SEQ ID NO: 57 - protein, 20 aa, Homo sapiens
  VARIANT 4, 16, 20: citrulline
HFSXASSIIDELFQDXFFTX

SEQ ID NO: 58 - protein, 20 aa, Homo sapiens
  VARIANT 6: citrulline
PVEVSXKNPKFMETVAEKAL

SEQ ID NO: 59 - protein, 20 aa, Homo sapiens
NSAQEDSDHDGQGDACDDDD

SEQ ID NO: 60 - protein, 20 aa, Homo sapiens
NDKARVEVERDNLAEDIMRL

SEQ ID NO: 61 - protein, 20 aa, Homo sapiens
EIQELQAQIQEQHVQIDVDV

SEQ ID NO: 62 - protein, 20 aa, Homo sapiens
MALDIEIATYRKLLEGEESR

SEQ ID NO: 63 - protein, 20 aa, Homo sapiens
LNLRETNLDSLPLVDTHSKR

SEQ ID NO: 64 - protein, 20 aa, Homo sapiens
THSKRTLLIKTVETRDGQVI

SEQ ID NO: 65 - protein, 20 aa, Homo sapiens
  VARIANT 8, 13: citrulline
GGPGTASXPSSSXSYVTTST

SEQ ID NO: 66 - protein, 20 aa, Homo sapiens
  VARIANT 7, 12, 14: citrulline
GGVYATXSSAVXLXSSVPGV

SEQ ID NO: 67 - protein, 20 aa, Homo sapiens
  VARIANT 4, 10, 20: citrulline
AANXNNDALXQAKQESTEYX

SEQ ID NO: 68 - protein, 20 aa, Homo sapiens
  VARIANT 4, 20: citrulline
LNLXETNLDSLPLVDTHSKX

SEQ ID NO: 69 - protein, 18 aa, Homo sapiens
TIHAHPGSRRGGRHGYHH

SEQ ID NO: 70 - protein, 18 aa, Homo sapiens
  VARIANT 9, 13: citrulline
TIHAHPGSXRGGXHGYHH

SEQ ID NO: 71 - protein, 19 aa, Homo sapiens
  VARIANT 13: citrulline
SHQESTRGRSRGXSGRSGS

SEQ ID NO: 72 - protein, 19 aa, Homo sapiens
  VARIANT 7, 16: citrulline
SHQESTXGRSRGRSGXSGS

SEQ ID NO: 73 - protein, 6 aa, Homo sapiens
PRTEIN

SEQ ID NO: 74 - protein, 20 aa, Homo sapiens
NQLLKLPVLPPKLTLFNAKY

SEQ ID NO: 75 - protein, 19 aa, Homo sapiens
PKRAVAREESGKPGAHVTV

SEQ ID NO: 76 - protein, 11 aa, Homo sapiens
VLNRLKVGLQV

SEQ ID NO: 77 - protein, 20 aa, Homo sapiens
RITEVWGIPSPIDTVFTRCN

SEQ ID NO: 78 - protein, 27 aa, Homo sapiens
DPMSSTYIEELGKREVTIPPKYRELLA

SEQ ID NO: 79 - protein, 20 aa, Homo sapiens
QTHMLDVMQDHFSRASSIID

SEQ ID NO: 80 - protein, 20 aa, Homo sapiens
LLSRLEELENLVSSLREQCT

SEQ ID NO: 81 - protein, 20 aa, Homo sapiens
  VARIANT 4, 16: citrulline
LLSXLEELENLVSSLXEQCT

SEQ ID NO: 82 - protein, 20 aa, Homo sapiens
VIISINGQSVVSANDVSDVI

SEQ ID NO: 83 - protein, 20 aa, Homo sapiens
VVRRGNEDIMITVIPEEIDP